(19) United States
(12) Patent Application Publication
(10) Pub. No.: US 2012/0142028 A1
(43) Pub. Date: Jun. 7, 2012

(54) BIOLOGICAL MARKERS PREDICTIVE OF ANTI-CANCER RESPONSE TO EPIDERMAL GROWTH FACTOR RECEPTOR KINASE INHIBITORS

(75) Inventors: Frank C. Richardson, Louisville, CO (US); G. David Young, Boulder, CO (US); Julie L. Wolf, Nederland, CO (US); Regina M. Sennello, Oak Park, IL (US)

(73) Assignee: OSI Pharmaceuticals, LLC

(21) Appl. No.: 13/264,786

(22) PCT Filed: Apr. 15, 2010
(86) PCT No.: PCT/US10/31144

§ 371 Date: Dec. 15, 2011

Related U.S. Application Data
(60) Provisional application No. 61/212,967, filed on Apr. 17, 2009.

(51) Int. Cl. G01N 33/566

(52) U.S. Cl. 435/7.21

(57) ABSTRACT

The present invention provides diagnostic methods for predicting the effectiveness of treatment of a cancer patient with an EGFR kinase inhibitor. These methods are based on the surprising discovery that the effectiveness of treatment with an EGFR kinase inhibitor is predicted by whether a patient's tumor cells express a high or a low level of the biomarkers vimentin and E-cadherin, such that patients whose tumors express a high level of at least one of the biomarkers vimentin and E-cadherin have a longer overall survival and progression free survival than patients whose tumors express a low level of both vimentin and E-cadherin. The present invention further provides a method for treating tumors or tumor metastases in a patient, comprising the steps of diagnosing a patient's likely responsiveness to an EGFR kinase inhibitor by assessing whether tumor cells express a high level of at least one of the biomarkers vimentin and E-cadherin, and administering to said patient a therapeutically effective amount of an EGFR kinase inhibitor (e.g. erlotinib), particularly when effectiveness of the inhibitor is predicted.

### BACKGROUND OF THE INVENTION

Cancer is a generic name for a wide range of cellular dysfunctions and dysregulations characterized by unregulated growth, lack of differentiation, and the potential to invade local tissues and metastasize to distant sites. These neoplastic malignancies may affect, with various degrees of prevalence, every tissue and organ in the body. The present invention is directed to methods for diagnosing and treating cancer patients. In particular, the present invention is directed to methods for determining which patients will most benefit from treatment with an epidermal growth factor receptor (EGFR) kinase inhibitor (e.g. erlotinib).

The epidermal growth factor receptor (EGFR) family comprises four closely related receptors (HER1/EGFR, HER2, HER3 and HER4) involved in cellular responses such as differentiation and proliferation. Over-expression of the EGFR kinase, or its ligand transforming growth factor-alpha (TGF-alpha), is frequently associated with many cancers, including breast, lung, colorectal, ovarian, renal cell, bladder, head and neck cancers, glioblastomas, and astrocytomas, and is believed to contribute to the malignant growth of these tumors. A specific deletion-mutation in the EGFR gene (EGFRvIII) has also been found to increase cellular tumorigenicity. Activation of EGFR stimulated signaling pathways promote multiple processes that are potentially cancer-promoting, e.g. proliferation, angiogenesis, cell motility and invasion, decreased apoptosis (programmed cell death) and induction of drug resistance. Increased HER1/EGFR expression is frequently linked to advanced disease, metastases and poor prognosis. For example, in non small cell lung cancer (NSCLC) and gastric cancer, increased HER1/EGFR expression has been shown to correlate with a high metastatic rate, poor tumor differentiation and increased tumor proliferation.

Mutations which activate the receptor's intrinsic protein tyrosine kinase activity and/or increase downstream signaling have been observed in NSCLC and glioblastoma. However the role of mutations as a principle mechanism in conferring sensitivity to EGFR inhibitors, for example erlotinib (TARCEVA®) or gefitinib (IRESSA™), has been controversial. Recently, a mutant form of the full length EGFR has been reported to predict responsiveness to the EGFR tyrosine kinase inhibitor gefitinib (Paez, J. G. et al. (2004) Science 304:1497-1500; Lynch, T. J. et al. (2004) N. Engl. J. Med. 350:2129-2139). Cell culture studies have shown that cell lines which express the mutant form of EGFR (i.e. H3255) were more sensitive to growth inhibition by the EGFR tyrosine kinase inhibitor gefitinib, and that much higher concentrations of gefitinib was required to inhibit the tumor cell lines expressing wild type EGFR. These observations suggests that specific mutant forms of EGFR may reflect a greater sensitivity to EGFR inhibitors, but do not identify a completely non-responsive phenotype.

The development for use as anti-tumor agents of compounds that directly inhibit the kinase activity of EGFR, as well as antibodies that reduce EGFR kinase activity by blocking EGFR activation, are areas of intense research effort (de Bono J. S. and Rowinsky, E. K. (2002) Trends in Mol. Medicine. 8:S19-S26; Dancey, J. and Sausville, E. A. (2003) Nature Rev. Drug Discovery 2:92-313). Several studies have demonstrated, disclosed, or suggested that some EGFR kinase inhibitors might improve tumor cell or neoplasia killing when used in combination with certain other anti-cancer or chemotherapeutic agents or treatments (e.g. Herbst, R. S. et al. (2001) Expert Opin. Biol. Ther. 1:719-732; Solomon, B. et al (2003) Int. J. Radiat. Oncol. Biol. Phys. 55:713-723; Krishnan, S. et al. (2003) Frontiers in Bioscience 8, e1-13; Grunwald, V. and Hidalgo, M. (2003) J. Nat. Cancer Inst. 95:851-867; Seymour L. (2003) Current Opin. Investig. Drugs 4(6):658-666; Khalil, M. Y. et al. (2003) Expert Rev. Anticancer Ther. 3:367-380; Bulgaru, A. M. et al. (2003) Expert Rev. Anticancer Ther. 3:269-279; Dancey, J. and Sausville, E. A. (2003) Nature Rev. Drug Discovery 2:92-313; Ciardiello, F. et al. (2000) Clin. Cancer Res. 6:2053-2063; and Patent Publication No: US 2003/0157104).

Erlotinib (e.g. erlotinib HCl, also known as TARCEVA® or OSI-774) is an orally available inhibitor of EGFR kinase. In vitro, erlotinib has demonstrated substantial inhibitory activity against EGFR kinase in a number of human tumor cell lines, including colorectal and breast cancer (Moyer J. D. et al. (1997) Cancer Res. 57:4838), and preclinical evaluation has demonstrated activity against a number of EGFR-expressing human tumor xenografts (Pollack, V. A. et al (1999) J. Pharmacol. Exp. Ther. 291:739). More recently, erlotinib has demonstrated promising activity in Phase I and II trials in a number of indications, including head and neck cancer (Soulieres, D., et al. (2004) J. Clin. Oncol. 22:77), NSCLC (Perez-Soler R, et al. (2001) Proc. Am. Soc. Clin. Oncol. 20:310a, abstract 1235), colorectal cancer (CRC) (Oza, M., et al. (2003) Proc. Am. Soc. Clin. Oncol. 22:196a, abstract 785) and metastatic breast cancer (MBC) (Winer, E., et al. (2002) Breast Cancer Res. Treat. 76:5115a, abstract 445). In a Phase III trial, erlotinib monotherapy significantly prolonged survival, delayed disease progression and delayed worsening of lung cancer-related symptoms in patients with advanced, treatment-refractory NSCLC (Shepherd, F. et al. (2004) J. Clin. Oncology, 22:14 S (July 15 Supplement), Abstract 7022). While most of the clinical trial data for erlotinib relate to its use in NSCLC, preliminary results from Phase I/II studies have demonstrated promising activity for erlotinib and capecitabine/erlotinib combination therapy in patients with wide range of human solid tumor types, including CRC (Oza, M., et al. (2003) Proc. Am. Soc. Clin. Oncol. 22:196a, abstract 785) and MBC (Jones, R. J., et al. (2003) Proc. Am. Soc. Clin. Oncol. 22:45a, abstract 180). In November 2004 the U.S. Food and Drug Administration (FDA) approved TARCEVA® for the treatment of patients with locally advanced or metastatic NSCLC after failure of at least one prior chemotherapy regimen. TARCEVA® is the only drug in EGFR class to demonstrate in a Phase III clinical trial an increase in survival in advanced NSCLC patients.

An anti-neoplastic drug would ideally kill cancer cells selectively, with a wide therapeutic index relative to its toxicity towards non-malignant cells. It would also retain its efficacy against malignant cells, even after prolonged exposure to the drug. Unfortunately, none of the current chemotherapies possess such an ideal profile. Instead, most possess very narrow therapeutic indexes. Furthermore, cancerous cells exposed to slightly sub-lethal concentrations of a chemotherapeutic agent will very often develop resistance to such an agent, and quite often cross-resistance to several other anti-cancer agents as well. Additionally, for any given cancer type one frequently cannot predict which patient is likely to respond to a particular treatment, even with newer gene-targeted therapies, such as EGFR kinase inhibitors, thus necessitating considerable trial and error, often at considerable risk and discomfort to the patient, in order to find the most effective therapy.

Thus, there is a need for more efficacious treatment for cancer and other proliferative disorders, and for more effective means for determining which tumors will respond to which treatment. Strategies for enhancing the therapeutic efficacy of existing drugs have involved changes in the schedule for their administration, and also their use in combination with other anti-cancer or biochemical modulating agents. Combination therapy is well known as a method that can result in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect is synergistic (the efficacy of the combination is greater than the sum of the effects of each drug given alone). Target-specific therapeutic approaches are generally associated with reduced toxicity compared with conventional cytotoxic agents, and therefore lend themselves to use in combination regimens.

Target-specific therapeutic approaches, such as erlotinib, are generally associated with reduced toxicity compared with conventional cytotoxic agents, and therefore lend themselves to use in combination regimens. Promising results have been observed in Phase I/II studies of erlotinib in combination with bevacizumab (Mininberg, E. D., et al. (2003) Proc. Am. Soc. Clin. Oncol. 22:627a, abstract 2521) and gemcitabine (Dragovich, T., (2003) Proc. Am. Soc. Clin. Oncol. 22:223a, abstract 895). Recent data in NSCLC Phase III trials have shown that first-line erlotinib or gefitinib in combination with standard chemotherapy did not improve survival (Gatzemeier, U., (2004) Proc. Am. Soc. Clin. Oncol. 23:617 (Abstract 7010); Herbst, R. S., (2004) Proc. Am. Soc. Clin. Oncol. 23:617 (Abstract 7011); Giaccone, G., et al. (2004) J. Clin. Oncol. 22:777; Herbst, R., et al. (2004) J. Clin. Oncol. 22:785). However, pancreatic cancer Phase III trials have shown that first-line erlotinib in combination with gemcitabine did improve survival (OSI Pharmaceuticals/Genentech/Roche Pharmaceuticals Press Release, Sep. 20, 2004).

Several groups have investigated potential biomarkers to predict a patient's response to EGFR inhibitors (see for example, PCT publications: WO 2004/063709, WO 2005/017493, WO 2004/111273, WO 2004/071572, WO 2005/117553, WO 2005/070020 and WO 2009/023172; and US published patent applications: US 2005/0019785, US 2004/0132097, US 2006/211060, US 2008/0090233, and US 2008/113874). However, diagnostic or prognostic tests are only now beginning to emerge that can guide practicing physicians in the treatment of their patients with EGFR kinase inhibitors, and there is a clear need for additional and improved tests.

During most cancer metastases, an important change occurs in a tumor cell known as the epithelial-mesenchymal transition (EMT) (Thiery, J. P. (2002) Nat. Rev. Cancer 2:442-454; Savagner, P. (2001) Bioessays 23:912-923; Kang Y. and Massague, J. (2004) Cell 118:277-279; Julien-Grille, S., et al. Cancer Research 63:2172-2178; Bates, R. C. et al. (2003) Current Biology 13:1721-1727; Lu Z., et al. (2003) Cancer Cell. 4(6):499-515)). EMT does not normally occur in healthy cells except during embryogenesis, though a transient EMT state is induced in epithelial wound healing to aid in the reconstruction of epithelial tissue. Epithelial cells, which are bound together tightly and exhibit polarity, change to a more mesenchymal cellular phenotype, in which these mesenchymal cells are held together more loosely, exhibit a loss of polarity, and have the ability to move within tissues. These mesenchymal-like cells can spread into tissues surrounding the original tumor, as well as separate from the tumor, invade blood and lymph vessels, and travel to new locations where they divide and form additional tumors. Recent research has demonstrated that epithelial cells respond well to EGFR and insulin-like growth factor-1 receptor (IGF-1R) kinase inhibitors, but that after an EMT the resulting mesenchymal-like tumor cells are much less sensitive to such inhibitors. (e.g. see Thompson, S. et al. (2005) Cancer Res. 65(20):9455-9462; U.S. Patent Application 60/997,514). Thus there is a pressing need for anti-cancer agents that can prevent or reverse tumor cell EMT events (e.g. stimulate a mesenchymal to epithelial transition (MET)), or inhibit the growth of the mesenchymal-like tumor cells resulting from EMT. Such agents should be particularly useful when used in conjunction with other anti-cancer drugs such as EGFR and IGF-1R kinase inhibitors.

As human cancers progress to a more invasive, metastatic state, multiple signaling programs regulating cell survival and migration are observed depending on cell and tissue contexts (Gupta, G. P., and Massague, J. (2006) Cell 127, 679-695). Recent data highlight the transdifferentiation of epithelial cancer cells to a more mesenchymal-like state, a process resembling epithelial-mesenchymal transition (EMT; (Oft, M., et al. (1996). Genes & development 10, 2462-2477; Perl, A. K., et al. (1998). Nature 392, 190-193), to facilitate cell invasion and metastasis (Brabletz, T. et al. (2005) Nat Rev Cancer 5, 744-749; Christofori, G. (2006) Nature 441, 444-450). Through EMT-like transitions mesenchymal-like tumor cells are thought to gain migratory capacity at the expense of proliferative potential. A mesenchymal-epithelial transition (MET) has been postulated to regenerate a more proliferative state and allow macrometastases resembling the primary tumor to form at distant sites (Thiery, J. P. (2002) Nat Rev Cancer 2, 442-454). EMT-like transitions in tumor cells result from transcriptional reprogramming over considerable periods of time (weeks to months) via transcription factors harboring zinc finger, forkhead, bHLH and HMG-box domains (Mani, S. A. et al. (2007) Proceedings of the National Academy of Sciences of the United States of America 104, 10069-10074; Peinado, H. et al. (2007) Nat Rev Cancer 7, 415-428). The loss of E-cadherin and transition to a more mesenchymal-like state, with increased expression of mesenchymal proteins such as vimentin or fibronectin, likely serves a major role in the progression of cancer (Matsumura, T. et al. (2001) Clin Cancer Res 7, 594-599; Yoshiura, K. et al. (1995). Proceedings of the National Academy of Sciences of the United States of America 92, 7416-7419) and the acquisition of a mesenchymal phenotype has been correlated with poor prognosis (Baumgart, E. et al. (2007) Clin Cancer Res 13, 1685-1694; Kokkinos, M. I. Et al. (2007) Cells, tissues, organs 185, 191-203; Willipinski-Stapelfeldt, B. et al. (2005) Clin Cancer Res 11, 8006-8014.). Targeting tumor-derived and/or tumor-associated stromal cells provides a unique mechanism to block EMT-like transitions and inhibit the survival of invading cells.

The cellular changes associated with EMT-like transitions alter the dependence of carcinoma cells on EGFR signaling networks for survival. It has been observed that an EMT-like transition was associated with cellular insensitivity to the EGFR kinase inhibitor erlotinib (Thomson, S. et al. (2005) Cancer Research 65, 9455-9462; Witta, S. E., et al. (2006) Cancer Research 66, 944-950; Yauch, R. L., et al. (2005) Clin Cancer Res 11, 8686-8698), in part from EGFR independent activation of either or both the PI3-kinase or Mek-Erk pathways (Buck, E. et al. (2007). Molecular Cancer Therapeutics 6, 532-541). Similar data correlating EMT status to sensitivity to EGFR kinase inhibitors have been reported in pancreatic, CRC (Buck, E. et al. (2007) Molecular Cancer Therapeutics 6, 532-541) bladder (Shrader, M. et al. (2007) Molecular Cancer Therapeutics 6, 277-285) and HNSCC (Frederick et al. (2007) Molecular Cancer Therapeutics 6, 1683-1691) cell lines, xenografts and in patients (Yauch, R. L., et al. (2005) Clin Cancer Res 11, 8686-8698). The molecular determinants to alternative routes of activation of the PI3-kinase and Erk pathways, which can bypass cellular sensitivity to EGFR inhibitors, have been actively investigated (Chakravarti, A. et al. (2002) Cancer research 62, 200-207; Engelman, J. A. et al. (2007) Science 316:1039-1043).

Thus, although considerable progress has been made in recent years in elucidating factors that influence tumor cell sensitivity to EGFR kinase inhibitors there remains a critical need for improved methods for determining the best mode of treatment for any given cancer patient and for the incorporation of such determinations into more effective treatment regimens for cancer patients, whether such inhibitors are used as single agents or combined with other anti-cancer agents.

### SUMMARY OF THE INVENTION

The present invention provides diagnostic and prognostic methods for predicting the effectiveness of treatment of a cancer patient with an EGFR kinase inhibitor. These methods are based on the surprising discovery that the effectiveness of treatment with an EGFR kinase inhibitor is predicted by whether a patient's tumor cells express a high or a low level of the biomarkers vimentin and E-cadherin, such that patients whose tumors express a high level of at least one of the biomarkers vimentin and E-cadherin have a longer overall survival and progression free survival than patients whose tumors express a low level of both vimentin and E-cadherin.

Improved methods for treating cancer patients with EGFR kinase inhibitors that incorporate the above methodology are also provided. Thus, the present invention further provides a method for treating tumors or tumor metastases in a patient, comprising the steps of diagnosing a patient's likely responsiveness to an EGFR kinase inhibitor by assessing whether tumor cells express a high level of at least one of the biomarkers vimentin and E-cadherin, and administering to said patient a therapeutically effective amount of an EGFR kinase inhibitor (e.g. erlotinib), particularly when effectiveness of the inhibitor is predicted.

### BRIEF DESCRIPTION OF THE FIGURES

FIG. 1: Representative examples of E-Cadherin staining intensities are

shown as follows: A. E-Cadherin +0; B. E-Cadherin +1; C. E-Cadherin +2; D. E-Cadherin +3.

FIG. 2: Representative examples of vimentin staining intensities are shown as follows: A. Vimentin +0; B. Vimentin +1; C. Vimentin +2; D. Vimentin +3.

FIG. 3: Kaplan-Meier Figures for Survival. This figure shows the overall survival analyses for E-Cadherin. The upper left plot compares the Erlotinib arm to the Placebo arm for the E-Cadherin high subset. The upper right plot compares the Erlotinib arm to the Placebo arm for the E-Cadherin low subset. The lower plots compare the E-Cadherin high subset to the E-Cadherin low subset for the Erlotinib arm (lower left plot) and for the Placebo arm (lower right plot). In the E-Cadherin high subset, the Erlotinib arm is more favorable, and similarly, for the Erlotinib arm, the E-Cadherin High subset is more favorable. For the E-Cadherin low subset and the Placebo arm, the effects are switched in direction but smaller in magnitude.

FIG. 4: Kaplan-Meier Figures for PFS. This figure shows the progression free survival analyses for E-Cadherin. The upper left plot compares the Erlotinib arm to the Placebo arm for the E-Cadherin high subset. The upper right plot compares the Erlotinib arm to the Placebo arm for the E-Cadherin low subset. The lower plots compare the E-Cadherin high subset to the E-Cadherin low subset for the Erlotinib arm (lower left plot) and for the Placebo arm (lower right plot). In the E-Cadherin high subset, the Erlotinib arm is more favorable, and similarly, for the Erlotinib arm, the E-Cadherin High subset is more favorable. For the E-Cadherin low subset and the Placebo arm, the effects are switched in direction but smaller in magnitude.

FIG. 5: Kaplan-Meier Figures for Survival. This figure shows the overall survival analyses for Vimentin. The upper left plot compares the Erlotinib arm to the Placebo arm for the vimentin high subset. The upper right plot compares the Erlotinib arm to the Placebo arm for the vimentin low subset. The lower plots compare the vimentin high subset to the vimentin low subset for the Erlotinib arm (lower left plot) and for the Placebo arm (lower right plot). In the vimentin high subset, the Erlotinib arm is more favorable, and similarly, for the Erlotinib arm, the vimentin high subset is more favorable. For the vimentin low subset there is no difference between Erlotinib and Placebo. In the Placebo arm, the effects are reversed, with the vimentin low subset more favorable than the vimentin high subset.

FIG. 6: Kaplan-Meier Figures for PFS. This figure shows the progression free survival analyses for Vimentin. The upper left plot compares the Erlotinib arm to the Placebo arm for the vimentin high subset. The upper right plot compares the Erlotinib arm to the Placebo arm for the vimentin low subset. The lower plots compare the vimentin high subset to the vimentin low subset for the Erlotinib arm (lower left plot) and for the Placebo arm (lower right plot). In the vimentin high subset, the Erlotinib arm is more favorable, and similarly, for the Erlotinib arm, the vimentin high subset is more favorable. For the vimentin low subset there is no difference between Erlotinib and Placebo. In the Placebo arm, the effects are reversed, with the vimentin low subset more favorable than the vimentin high subset.

FIG. 7: Response Rates. This table reports the Response rates (Complete Response+Partial Response) and the Disease Control Rate (Complete Response+Partial Response+Stable Disease) for specified subsets of the BR.21 patients. The proportion reported is the number of patients (n) with CR+PR or CR+PR+SD, divided by the number of patients in the subset (N). This is presented as a fraction (n/N) and a percentage with 95% exact confidence limits for each treatment arm. P-values (from Fisher's Exact Test) are provided to test for differences in the response rates of the Erlotinib and Placebo arms.

FIG. 8: E-Cadherin Staining of Intensity +2 or +3: By Treatment Arm, Comparing High vs. Low.

FIG. 9: E-Cadherin Staining of Intensity +2 or +3: By Treatment Arm, Comparing High vs. Low.

FIG. 10: E-Cadherin Staining of Any Intensity: By Treatment Arm, Comparing High vs. Low.

FIG. 11: E-Cadherin Staining of Any Intensity: By E-Cadherin Status, Comparing Erlotinib vs. Placebo.

FIG. 12: E-Cadherin Composite Score: By Treatment Arm, Comparing High vs. Low.

FIG. 13: E-Cadherin Composite Score: By E-Cadherin Status, Comparing Erlotinib vs. Placebo.

FIG. 14: Vimentin Staining of Any Intensity: By Treatment Arm, Comparing High vs. Low.

FIG. 15: Vimentin Staining of Any Intensity: By vimentin Status, Comparing Erlotinib vs. Placebo.

FIG. 16: Vimentin Staining of Intensity +2 or +3: By Treatment Arm, Comparing High vs. Low.

FIG. 17: Vimentin Staining of Intensity +2 or +3: By vimentin Status, Comparing Erlotinib vs. Placebo.

FIG. 18: Vimentin Composite Score: By Treatment Arm, Comparing High vs. Low.

FIG. 19: Vimentin Composite Score: By vimentin Status, Comparing Erlotinib vs. Placebo.

### DETAILED DESCRIPTION OF THE INVENTION

The term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

“Cell growth”, as used herein, for example in the context of “tumor cell growth”, unless otherwise indicated, is used as commonly used in oncology, where the term is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

“Tumor growth” or “tumor metastases growth”, as used herein, unless otherwise indicated, is used as commonly used in oncology, where the term is principally associated with an increased mass or volume of the tumor or tumor metastases, primarily as a result of tumor cell growth.

The term “treating” as used herein, unless otherwise indicated, means reversing, alleviating, inhibiting the progress of, or preventing, either partially or completely, the growth of tumors, tumor metastases, or other cancer-causing or neoplastic cells in a patient with cancer. The term “treatment” as used herein, unless otherwise indicated, refers to the act of treating.

The phrase “a method of treating” or its equivalent, when applied to, for example, cancer refers to a procedure or course of action that is designed to reduce or eliminate the number of cancer cells in an individual, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound or combination that will elicit the biological or medical response of a tissue, system, animal or human that is being sought by the researcher, veterinarian, medical doctor or other clinician.

The data presented in the Examples herein below demonstrate that the effectiveness of treatment of a cancer patient with an EGFR kinase inhibitor is predicted by whether the patient's tumor cells express a high or a low level of the biomarkers vimentin and E-cadherin, such that patients whose tumors express a high level of at least one of the biomarkers vimentin or E-cadherin have a longer overall survival and progression free survival in response to treatment than patients whose tumors express a low level of both of these biomarkers. A high level of either one of the biomarkers predicts effectiveness of treatment, even when the level of the other biomarker is low. Thus, for example, treatment is predicted to be effective if vimentin expression level is high, even if E-cadherin expression levels are low. These observations are the basis of valuable new diagnostic methods for predicting the effects of EGFR kinase inhibitors on patient outcome, and give oncologists an additional tool to assist them in choosing the most appropriate treatment regimen for their patients.

This vimentin biomarker result is extremely surprising given the considerable body of work that suggests that high vimentin expression in tumor cells (i.e. after EMT) correlates with reduced sensitivity of tumor cell growth to inhibition by EGFR kinase inhibitors (e.g. see Thomson, S. et al. (2005) Cancer Research 65, 9455-9462). The reason for the apparent lack of concordance between the earlier studies in cell lines and animal models and the clinical studies described herein is presently unknown.

Accordingly, the present invention provides a method of predicting the effectiveness of treatment of a cancer patient with an EGFR kinase inhibitor, comprising: assessing the level of the biomarker vimentin expressed by cells of a tumor of the patient; assessing the level of the biomarker E-cadherin expressed by cells of the same tumor; determining whether the tumor expresses high or low expression levels of the two biomarkers, by, for example, comparison to a reference level or a control sample, or by using a standardized scoring system; and predicting the effectiveness of treatment, wherein a high level of at least one of the two biomarkers indicates that treatment will be more effective; and wherein the effectiveness of treatment of the cancer patient is indicated by either a longer overall survival or longer progression free survival in response to treatment.

The present invention also provides a method of predicting the effectiveness of treatment of a cancer patient with an EGFR kinase inhibitor, comprising: assessing the level of the biomarker vimentin expressed by cells of a tumor of the patient; determining whether the tumor expresses high or low expression levels of vimentin, by, for example, comparison to a reference level or a control sample, or by using a standardized scoring system; and predicting the effectiveness of treatment, wherein a high level of vimentin indicates that treatment will be more effective; and wherein the effectiveness of treatment of the cancer patient is indicated by either a longer overall survival or longer progression free survival in response to treatment.

The present invention also provides a method of predicting the effectiveness of treatment of a cancer patient with an EGFR kinase inhibitor, comprising: assessing the level of the biomarker E-cadherin expressed by cells of a tumor of the patient; determining whether the tumor expresses high or low expression levels of E-cadherin, by, for example, comparison to a reference level or a control sample, or by using a standardized scoring system; and predicting the effectiveness of treatment, wherein a high level of E-cadherin indicates that treatment will be more effective; and wherein the effectiveness of treatment of the cancer patient is indicated by either a longer overall survival or longer progression free survival in response to treatment.

Inclusion of any of the biomarker diagnostic methods described herein as part of treatment regimens to predict the effectiveness of treatment of a cancer patient with an EGFR kinase inhibitor provides an advantage over treatment regiments that do not include such a biomarker diagnostic step, in that only that patient population which derives most benefit from an EGFR kinase inhibitor need be treated, and in particular, patients who are predicted not to benefit from treatment with an EGFR kinase inhibitor need not be treated.

Methods of this invention that measure both E-cadherin and vimentin biomarkers can provide potentially superior results to diagnostic assays measuring just one of these biomarkers, as illustrated by the data presented herein. For example, a diagnostic method that measures just E-cadherin would fail to predict effectiveness of EGFR kinase inhibitor treatment in the patient population whose tumor expresses low E-cadherin, but also expresses high vimentin (˜14% of patients in the study reported herein). A dual vimentin/E-cadherin biomarker approach thus reduces the number of patients that are predicted not to benefit from treatment with an EGFR kinase inhibitor, and thus potentially reduces the number of patients that fail to receive treatment that may extend their life significantly.

The present invention further provides a method for treating a patient with cancer, comprising the step of diagnosing a patient's likely responsiveness to an EGFR kinase inhibitor by any of the methods of the invention described herein for predicting effectiveness of an EGFR kinase inhibitor; and a step of administering the patient a therapeutically effective dose of an EGFR kinase inhibitor.

The present invention provides a method for treating a patient with cancer, comprising: a step of predicting the effectiveness of treatment of a cancer patient with an EGFR kinase inhibitor, by assessing the level of the biomarker vimentin expressed by cells of a tumor of the patient; assessing the level of the biomarker E-cadherin expressed by cells of the same tumor; determining whether the tumor expresses high or low expression levels of the two biomarkers, by, for example, comparison to a reference level or a control sample, or by using a standardized scoring system; and predicting the effectiveness of treatment, wherein a high level of at least one of the two biomarkers indicates that treatment will be more effective; and wherein the effectiveness of treatment of the cancer patient is indicated by either a longer overall survival or longer progression free survival in response to treatment; and a step of administering the patient a therapeutically effective dose of an EGFR kinase inhibitor.

The present invention also provides a method for treating a patient with cancer, comprising: a step of predicting the effectiveness of treatment of a cancer patient with an EGFR kinase inhibitor, by assessing the level of the biomarker vimentin expressed by cells of a tumor of the patient; determining whether the tumor expresses high or low expression levels of vimentin, by, for example, comparison to a reference level or a control sample, or by using a standardized scoring system; and predicting the effectiveness of treatment, wherein a high level of vimentin indicates that treatment will be more effective; and wherein the effectiveness of treatment of the cancer patient is indicated by either a longer overall survival or longer progression free survival in response to treatment; and a step of administering the patient a therapeutically effective dose of an EGFR kinase inhibitor.

The present invention also provides a method for treating a patient with cancer, comprising: a step of predicting the effectiveness of treatment of a cancer patient with an EGFR kinase inhibitor, by assessing the level of the biomarker E-cadherin expressed by cells of a tumor of the patient; determining whether the tumor expresses high or low expression levels of E-cadherin, by, for example, comparison to a reference level or a control sample, or by using a standardized scoring system; and predicting the effectiveness of treatment, wherein a high level of E-cadherin indicates that treatment will be more effective; and wherein the effectiveness of treatment of the cancer patient is indicated by either a longer overall survival or longer progression free survival in response to treatment; and a step of administering the patient a therapeutically effective dose of an EGFR kinase inhibitor.

The present invention also provides a method for treating a patient with cancer, comprising administering to the patient a therapeutically effective dose of an EGFR kinase inhibitor if it is predicted that the patient will have a longer overall survival or longer progression free survival in response to the treatment by virtue of having tumor cells that express high levels of the biomarker E-cadherin.

In one embodiment of any of the methods of treating a patient described herein, the step of administering the patient a therapeutically effective dose of an EGFR kinase inhibitor is conditional on the prior biomarker diagnostic step indicating that treatment will be more effective (i.e. E-cadherin and/or vimentin expression levels in tumor cells are high). In an alternative embodiment of any of the methods of treating a patient described herein, the patient is administered a therapeutically effective dose of an EGFR kinase inhibitor even when the prior biomarker diagnostic step predicts that treatment is not likely to be particularly effective (e.g. both E-cadherin and vimentin expression levels in tumor cells are low). The latter embodiment may be pursued if, for example, in a physicians judgment some benefit may still be achieved by administration of an EGFR kinase inhibitor, and/or other options for the patient are limited or non-existent.

For the methods of treatment described herein, an example of a preferred EGFR kinase inhibitor is erlotinib, including pharmacologically acceptable salts or polymorphs thereof. One or more additional anti-cancer agents or treatments may also be co-administered simultaneously or sequentially with the EGFR kinase inhibitor, as judged to be appropriate by the administering physician given the prediction of the likely responsiveness of the patient to an EGFR kinase inhibitor, in combination with any additional circumstances pertaining to the individual patient.

Thus, it will be appreciated by one of skill in the medical arts that the exact manner of administering to said patient a therapeutically effective amount of an EGFR kinase inhibitor following a diagnosis of a patient's likely responsiveness to an EGFR kinase inhibitor will be at the discretion of the attending physician. The mode of administration, including dosage, combination with other anti-cancer agents, timing and frequency of administration, and the like, may be affected by the diagnosis of a patient's likely responsiveness to an EGFR kinase inhibitor, as well as the patient's condition and history. Thus, even patients that are diagnosed to not respond well to EGFR kinase inhibitors may still benefit from treatment with such inhibitors, particularly in combination with other anti-cancer agents, or agents that may alter a patient's response to EGFR kinase inhibitors.

In the methods of this invention the terms “high” or “low” when referring to biomarker expression levels indicate whether the expression level is above or below a cut-point level that separates patient tumor expression levels into two ranges of expression levels that define two groups of patients who respond differently to treatment with an EGFR kinase inhibitor (e.g. erlotinib), i.e. the group with high expression of vimentin or E-cadherin responding more effectively to treatment than the group with low expression.

For example, in one embodiment, for vimentin, wherein vimentin protein expression is determined by immunohistochemistry (IHC), a cut-point level of 10% of tumor cells expressing any level of vimentin (i.e. staining intensity of +1, +2, or +3) is chosen, such that high vimentin is when 10% or more of the cells express any level of vimentin. Thus, in this embodiment, any tumor sample in which at least 10% of the tumor cells (e.g. 10%, 20%, 30%, 40%, 50%, 60%, 70%, 80%, 90%, or 100%, or any intermediate value between these values) express any level of vimentin is considered to express high vimentin. Low vimentin in this embodiment is thus when less than 10% of the tumor cells express any level of vimentin.

In another embodiment, for E-cadherin, wherein E-cadherin protein expression is determined by immunohistochemistry (IHC), a cut-point level of 40% of tumor cells with E-cadherin staining intensity of +2 or +3 is chosen, such that high E-cadherin is when 40% or more of the cells express E-cadherin with staining intensity of +2 or +3. Thus, in this embodiment, any tumor sample in which at least 40% of the tumor cells (e.g. 40%, 50%, 60%, 70%, 80%, 90%, or 100%, or any intermediate value between these values) express E-cadherin with a staining intensity of +2 or +3 is considered to express high E-cadherin. Low E-cadherin in this embodiment is thus when less than 40% of the tumor cells express E-cadherin with a staining intensity of +2 or +3 (e.g. 30%, 20%, 10%, 0%, or any intermediate value between these values).

It will be appreciated by one of skill in the art that analogous cut-point levels can be established for other embodiments of the methods described herein. For example, when alternative methods are employed to measure protein biomarker expression, or when mRNA level is assessed to determine biomarker expression level, suitable levels can readily be chosen that allow classification of biomarker levels as high or low, and similarly define two groups of patients who respond differently to treatment with an EGFR kinase inhibitor.

In any of the methods of this invention, the terms “reference level” or “control sample” are used to refer to standards that can be used for comparison in the determination of the expression level of a biomarker in order to assess whether a test sample level is high or low. A suitable standard may for example be a tumor sample from the experimental work described herein, or a comparable study, which gives the investigator a sense of the range of expression levels that occurs within the patient population, and thus enables one to know where a test sample expression level falls within that range (i.e. high or low).

In any of the methods of this invention, the term “standardized scoring system” is used to refer to a system of quantifying biomarker expression levels that can be used to ensure assay reproducibility from one experiment to the next, or from one investigator to the next, such that assessment of any test sample biomarker levels using such a system can readily be related to past results, and for example expression levels determined to be either high or low. The scoring system described herein in the experimental section is an example of such a standardized scoring system for use in immunohistochemistry (IHC). Alternative scoring or quantitation systems for IHC which could also be employed are known in the art.

In any of the methods of the invention described herein, the step of “assessing the level of a biomarker (e.g. E-cadherin, vimentin) expressed by cells of a tumor of the patient” may encompass additional steps, such as for example one or more of the following steps: 1. Obtaining a sample of the tumor from the cancer patient; 2. Contacting a sample of the tumor with an anti-biomarker antibody, or a biomarker probe; and 3. Employing a detection method (e.g. chromogenic; fluorescent) to localize and quantify the sites of anti-biomarker antibody or probe binding in the sample of the tumor.

Assessment of the expression level of vimentin or E-cadherin biomarker in a patient's tumor cells as high or low in any of the methods of this invention may be determined by comparison to the expression level of said biomarkers in a control tumor cell sample as a reference level, wherein this control tumor cell biomarker level has been previously correlated with a patient's responsiveness to treatment with an EGFR kinase inhibitor. Alternatively, a panel of such patient tumor cell samples, representing a range of biomarker expression levels from low to high, and thus a range of patients' responsiveness to treatment with an EGFR kinase inhibitor, can be used construct a standard curve from which responsiveness to an EGFR kinase inhibitor can be predicted from the biomarker expression levels of test tumor cell samples. Alternatively, a standardized scoring system can be used to determine whether the expression level of vimentin or E-cadherin biomarker in a patient's tumor cells is high or low.

Expression levels of a biomarker in a test tumor cell sample, or a control cell sample, may be determined relative to cell number, total protein or total RNA level, or the expression level of a housekeeping gene whose expression varies little or not at all from one cell to another (e.g. GAPDH, (3-actin, tubulin, or the like), to give a “relative expression level.”. Comparison of biomarker expression levels in a test tumor cell sample versus a control cell sample may be performed by comparing such relative expression levels.

It will be appreciated by those of skill in the art that a control cell sample need not be established for each new assay, at the time the assay is performed, but rather a baseline or control can be established by referring to a form of stored information regarding a previously determined control level for treated patients (including both groups who respond favorably and less favorably to EGFR kinase inhibitor treatment), such as a control level established by any of the methods described herein. Such a form of stored information can include, for example, but is not limited to, a reference chart, listing or electronic file of population or individual data regarding favorably and less favorably responding patients, or any other source of data regarding control levels of expression biomarkers that is useful for the patient to be evaluated.

The present invention also provides a method of predicting whether treatment of a cancer patient with an EGFR kinase inhibitor will lead to longer overall survival or longer progression free survival, comprising: measuring the level of the biomarker vimentin expressed by cells of a tumor of the patient; measuring the level of the biomarker E-cadherin expressed by cells of a tumor of the patient; and determining whether the tumor expresses at least one of vimentin or E-cadherin at or above a cut-point level, at or above which it has been shown that longer overall survival or longer progression free survival results on treatment with an EGFR kinase inhibitor. In one embodiment of this method, the level of the biomarkers is determined by immunohistochemical determination of protein expression, and the cut-point level for vimentin is 10% of tumor cells expressing any level of vimentin protein (i.e. a staining intensity of +1 or greater), and the cut-point level for E-cadherin is 40% of tumor cells expressing E-cadherin protein with a staining intensity of at least +2.

The present invention also provides a method of predicting whether treatment of a cancer patient with an EGFR kinase inhibitor will lead to longer overall survival or longer progression free survival, comprising: measuring the level of the biomarker vimentin expressed by cells of a tumor of the patient; and determining whether the tumor expresses vimentin at or above a cut-point level, at or above which it has been shown that longer overall survival or longer progression free survival results on treatment with an EGFR kinase inhibitor. In one embodiment of this method, the level of vimentin biomarker is determined by immunohistochemical determination of vimentin protein expression, and the cut-point level is 10% of tumor cells expressing any level of vimentin protein (i.e. a staining intensity of +1 or greater).

The present invention also provides a method of predicting whether treatment of a cancer patient with an EGFR kinase inhibitor will lead to longer overall survival or longer progression free survival, comprising: measuring the level of the biomarker E-cadherin expressed by cells of a tumor of the patient; and determining whether the tumor expresses E-cadherin at or above a cut-point level, at or above which it has been shown that longer overall survival or longer progression free survival results on treatment with an EGFR kinase inhibitor. In one embodiment of this method, the level of E-cadherin biomarker is determined by immunohistochemical determination of E-cadherin protein expression, and the cut-point level is 40% of tumor cells expressing E-cadherin protein with a staining intensity of at least +2.

The present invention further provides a method for treating a patient with cancer, comprising: a step of predicting whether treatment of a cancer patient with an EGFR kinase inhibitor will lead to longer overall survival or longer progression free survival, comprising: measuring the level of the biomarker vimentin expressed by cells of a tumor of the patient; measuring the level of the biomarker E-cadherin expressed by cells of a tumor of the patient; and determining whether the tumor expresses at least one of vimentin or E-cadherin at or above a cut-point level, at or above which it has been shown that longer overall survival or longer progression free survival results on treatment with an EGFR kinase inhibitor; and a step of administering the patient a therapeutically effective dose of an EGFR kinase inhibitor. In one embodiment of this method, the level of the biomarkers is determined by immunohistochemical determination of protein expression, and the cut-point level for vimentin is 10% of tumor cells expressing any level of vimentin protein (i.e. a staining intensity of +1 or greater), and the cut-point level for E-cadherin is 40% of tumor cells expressing E-cadherin protein with a staining intensity of at least +2.

The present invention further provides a method for treating a patient with cancer, comprising: a step of predicting whether treatment of a cancer patient with an EGFR kinase inhibitor will lead to longer overall survival or longer progression free survival, comprising: measuring the level of the biomarker vimentin expressed by cells of a tumor of the patient; and determining whether the tumor expresses vimentin at or above a cut-point level, at or above which it has been shown that longer overall survival or longer progression free survival results on treatment with an EGFR kinase inhibitor; and a step of administering the patient a therapeutically effective dose of an EGFR kinase inhibitor. In one embodiment of this method, the level of vimentin biomarker is determined by immunohistochemical determination of vimentin protein expression, and the cut-point level is 10% of tumor cells expressing any level of vimentin protein (i.e. a staining intensity of +1 or greater).

The present invention further provides a method for treating a patient with cancer, comprising: a step of predicting whether treatment of a cancer patient with an EGFR kinase inhibitor will lead to longer overall survival or longer progression free survival, comprising: measuring the level of the biomarker E-cadherin expressed by cells of a tumor of the patient; and determining whether the tumor expresses E-cadherin at or above a cut-point level, at or above which it has been shown that longer overall survival or longer progression free survival results on treatment with an EGFR kinase inhibitor; and a step of administering the patient a therapeutically effective dose of an EGFR kinase inhibitor. In one embodiment of this method, the level of E-cadherin biomarker is determined by immunohistochemical determination of E-cadherin protein expression, and the cut-point level is 40% of tumor cells expressing E-cadherin protein with a staining intensity of at least +2.

The present invention further provides a method of identifying patients with cancer who are most likely to benefit from treatment with an EGFR kinase inhibitor, comprising: obtaining a sample of the patient's tumor, determining if tumor cells of the sample express a high level of vimentin biomarker, and identifying the patient as likely to benefit from treatment with an EGFR kinase inhibitor if high levels of vimentin biomarker are found. In one embodiment of this method, the level of vimentin biomarker is determined by immunohistochemical determination of vimentin protein expression. In another embodiment of this method the level of vimentin biomarker is determined by assessing the level of vimentin mRNA. In one embodiment of these methods, vimentin biomarker level is high if 10% or more of tumor cells express any level of vimentin biomarker (e.g. a staining intensity of +1 or greater by IHC, for vimentin protein). In one embodiment of these methods the benefit from treatment is indicated by either a longer overall survival or longer progression free survival in response to treatment. In another embodiment of these methods the benefit from treatment is indicated by another biological or medical response that indicates that treatment is effective, e.g. tumor regression, reduced levels of tumor markers in blood samples.

The present invention further provides a method of identifying patients with cancer who are most likely to benefit from treatment with an EGFR kinase inhibitor, comprising: obtaining a sample of the patient's tumor, determining if tumor cells of the sample express a high level of E-cadherin biomarker, and identifying the patient as likely to benefit from treatment with an EGFR kinase inhibitor if high levels of E-cadherin biomarker are found. In one embodiment of this method, the level of E-cadherin biomarker is determined by immunohistochemical determination of E-cadherin protein expression. In one embodiment of this method, E-cadherin biomarker protein level is high if 40% or more of tumor cells express E-cadherin protein with a staining intensity of at least +2 by IHC. In another embodiment the level of E-cadherin biomarker is determined by assessing the level of E-cadherin mRNA. In one embodiment of these methods the benefit from treatment is indicated by either a longer overall survival or longer progression free survival in response to treatment. In another embodiment of these methods the benefit from treatment is indicated by another biological or medical response that indicates that treatment is effective, e.g. tumor regression, reduced levels of tumor markers in blood samples.

The present invention further provides a method of identifying patients with cancer who are most likely to benefit from treatment with an EGFR kinase inhibitor, comprising: obtaining a sample of the patient's tumor, determining if tumor cells of the sample express a high level of vimentin biomarker, determining if tumor cells of the sample express a high level of E-cadherin biomarker, and identifying the patient as likely to benefit from treatment with an EGFR kinase inhibitor if high levels of vimentin biomarker and/or E-cadherin biomarker are found (i.e. high levels of at least one of these biomarkers). In one embodiment of this method, the level of vimentin biomarker or E-cadherin biomarker is determined by immunohistochemical determination of vimentin protein expression. In another embodiment of this method the level of vimentin biomarker or E-cadherin biomarker is determined by assessing the level of vimentin mRNA. In one embodiment of these methods, vimentin biomarker level is high if 10% or more of tumor cells express any level of vimentin biomarker (e.g. a staining intensity of +1 or greater by IHC, for vimentin protein). In one embodiment of these methods, E-cadherin biomarker protein level is high if 40% or more of tumor cells express E-cadherin protein with a staining intensity of at least +2 by IHC. In one embodiment of these methods the benefit from treatment is indicated by either a longer overall survival or longer progression free survival in response to treatment. In another embodiment of these methods the benefit from treatment is indicated by another biological or medical response that indicates that treatment is effective, e.g. tumor regression, reduced levels of tumor markers in blood samples.

The present invention further provides a method for treating a patient with cancer comprising a diagnostic step that determines whether the patient with cancer is one who is most likely to benefit from treatment with an EGFR kinase inhibitor, using any of the preceding methods, and a treatment step of administering the patient a therapeutically effective dose of an EGFR kinase inhibitor, particularly if the patient is found to have high tumor cell levels of at least one of the biomarkers vimentin and E-cadherin.

The present invention thus provides a method for treating a patient with cancer, comprising: a step of identifying patients with cancer who are most likely to benefit from treatment with an EGFR kinase inhibitor, by obtaining a sample of the patient's tumor, determining if tumor cells of the sample express a high level of vimentin biomarker, and identifying the patient as likely to benefit from treatment with an EGFR kinase inhibitor if high levels of vimentin biomarker are found, and a step of administering to the patient a therapeutically effective dose of an EGFR kinase inhibitor.

The present invention thus provides a method for treating a patient with cancer, comprising: a step of identifying patients with cancer who are most likely to benefit from treatment with an EGFR kinase inhibitor, by obtaining a sample of the patient's tumor, determining if tumor cells of the sample express a high level of E-cadherin biomarker, and identifying the patient as likely to benefit from treatment with an EGFR kinase inhibitor if high levels of E-cadherin biomarker are found, and a step of administering to the patient a therapeutically effective dose of an EGFR kinase inhibitor.

The present invention thus provides a method for treating a patient with cancer, comprising: a step of identifying patients with cancer who are most likely to benefit from treatment with an EGFR kinase inhibitor, by obtaining a sample of the patient's tumor, determining if tumor cells of the sample express a high level of vimentin biomarker, determining if tumor cells of the sample express a high level of E-cadherin biomarker, and identifying the patient as likely to benefit from treatment with an EGFR kinase inhibitor if high levels of vimentin biomarker and/or E-cadherin biomarker are found (i.e. high levels of at least one of these biomarkers), and a step of administering to the patient a therapeutically effective dose of an EGFR kinase inhibitor.

The biomarker E-cadherin is a product (protein or mRNA) expressed by the gene with NCBI GeneID 999. An example of a protein sequence expressed by the E-cadherin gene is NCBI RefSeq (Reference Sequence) NP_{—}004351. The biomarker vimentin is a product (protein or mRNA) expressed by the gene with NCBI GeneID 7431. An example of a protein sequence expressed by the vimentin gene is NCBI RefSeq (Reference Sequence) NP_{—}003371. The NCBI GeneID numbers listed herein are unique identifiers of the human gene from the NCBI Entrez Gene database record (National Center for Biotechnology Information (NCBI), U.S. National Library of Medicine, 8600 Rockville Pike, Building 38A, Bethesda, Md. 20894; Internet address http://www.ncbi.nlm.nih.gov/). They are used herein to unambiguously identify gene products that are referred to elsewhere in the application by names and/or acronyms. Proteins expressed by genes thus identified represent proteins that may be used in the methods of this invention, and the sequences of these proteins, including different isoforms, as disclosed in NCBI database (e.g. GENBANK®) records are herein incorporated by reference.

In the methods of this invention, the tumor cell of the cancer patient is preferably of a type known to, or expected to, express EGFR kinase, as do most tumor cells from solid tumors derived from epithelial cell linage. Such tumor cells include those from, for example, lung cancer tumors (e.g. non-small cell lung cancer (NSCLC)), pancreatic cancer tumors, breast cancer tumors, head and neck cancer tumors, gastric cancer tumors, colon cancer tumors, ovarian cancer tumors, or a tumor cell from any of a variety of other cancers as described herein below. The EGFR kinase of these tumor cells can be wild type or a mutant form.

In the methods of this invention, the EGFR kinase inhibitor can be any EGFR kinase inhibitor as described herein below. In one embodiment the EGFR kinase inhibitor is 6,7-bis(2-methoxyethoxy)-4-quinazolin-4-yl]-(3-ethynylphenyl)amine (also known as erlotinib, OSI-774, or TARCEVA® (i.e. erlotinib HCl), including pharmacologically acceptable salts or polymorphs thereof.

In the methods of this invention, the expression level of a tumor cell biomarker is preferably assessed by assaying a tumor biopsy. However, in an alternative embodiment, expression level of the tumor cell biomarker can be assessed in bodily fluids or excretions containing detectable levels of biomarkers originating from the tumor or tumor cells. Bodily fluids or excretions useful in the present invention include blood, urine, saliva, stool, pleural fluid, lymphatic fluid, sputum, ascites, prostatic fluid, cerebrospinal fluid (CSF), or any other bodily secretion or derivative thereof. By blood it is meant to include whole blood, plasma, serum or any derivative of blood. Assessment of tumor biomarkers in such bodily fluids or excretions can sometimes be preferred in circumstances where an invasive sampling method is inappropriate or inconvenient. For assessment of tumor cell biomarker expression, patient samples containing tumor cells, or proteins or nucleic acids produced by these tumor cells, may be used in the methods of the present invention. In these embodiments, the level of expression of the biomarker can be assessed by assessing the amount (e.g. absolute amount or concentration) of the marker in a tumor cell sample, e.g., a tumor biopsy obtained from a patient, or other patient sample containing material derived from the tumor (e.g. blood, serum, urine, or other bodily fluids or excretions as described herein above). The cell sample can, of course, be subjected to a variety of well-known post-collection preparative and storage techniques (e.g., nucleic acid and/or protein extraction, fixation, storage, freezing, ultrafiltration, concentration, evaporation, centrifugation, etc.) prior to assessing the amount of the marker in the sample. Likewise, tumor biopsies may also be subjected to post-collection preparative and storage techniques, e.g., fixation.

In the methods of this invention, the level of a biomarker expressed by a tumor cell can be assessed by using any of the standard bioassay procedures known in the art for determination of the level of expression of a gene, including for example immunohistochemistry (IHC), enzyme-linked immunosorbent assay (ELISA), radioimmunoassay (RIA), immunoprecipitation, immunoblotting, immunofluorescence microscopy, real-time polymerase chain reaction (RT-PCR), in situ hybridization, cDNA microarray, or the like, as described in more detail below. Expression of biomarker protein may be assessed by any of a wide variety of well known methods for detecting expression of a transcribed protein. Non-limiting examples of such methods include immunological methods for detection of secreted, cell-surface, cytoplasmic, or nuclear proteins, protein purification methods, or protein function or activity assays. Expression of a biomarker mRNA may be assessed by any of a wide variety of well known methods for detecting expression of a transcribed nucleic acid. Non-limiting examples of such methods include nucleic acid hybridization methods, nucleic acid reverse transcription methods, and nucleic acid amplification methods.

In one embodiment, expression of a biomarker protein is assessed using an antibody (e.g. a radio-labeled, chromophore-labeled, fluorophore-labeled, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a biomarker protein or fragment thereof, including a biomarker protein which has undergone either all or a portion of post-translational modifications to which it is normally subjected in the tumor cell (e.g. glycosylation, phosphorylation, methylation etc.).

Examples of suitable antibodies for performing the methods of the invention include the following specific antibodies: A. Antibodies that bind to human E-cadherin: e.g. clones 24E10 (Cell Signaling Technology (CST)); or NCH-38 (Dako); and B. Antibodies that bind to human vimentin: e.g. clone V9 (sold by Dako, Biocare, Vector Laboratories or Zymed); SP20 (sold by Lab Vision/Neomarkers or Vector Laboratories); or 3B4 (sold by LabVision/Neomarkers).

In another embodiment, expression of a biomarker is assessed by preparing mRNA/cDNA (i.e. a transcribed polynucleotide) from cells in a patient sample, and by hybridizing the mRNA/cDNA with a reference polynucleotide which is a complement of a biomarker nucleic acid, or a fragment thereof. cDNA can, optionally, be amplified using any of a variety of polymerase chain reaction methods prior to hybridization with the reference polynucleotide. Expression of one or more biomarkers can likewise be detected using quantitative PCR to assess the level of expression of the biomarker(s). Alternatively, any of the many known methods of detecting mutations or variants (e.g. single nucleotide polymorphisms, deletions, etc.) of a biomarker of the invention may be used to detect occurrence of a biomarker in a patient.

In a related embodiment, a mixture of transcribed polynucleotides obtained from the sample is contacted with a substrate having fixed thereto a polynucleotide complementary to or homologous with at least a portion (e.g. at least 7, 10, 15, 20, 25, 30, 40, 50, 100, 500, or more nucleotide residues) of a biomarker nucleic acid. If polynucleotides complementary to or homologous with are differentially detectable on the substrate (e.g. detectable using different chromophores or fluorophores, or fixed to different selected positions), then the levels of expression of a plurality of biomarkers can be assessed simultaneously using a single substrate (e.g. a “gene chip” microarray of polynucleotides fixed at selected positions). When a method of assessing biomarker expression is used which involves hybridization of one nucleic acid with another, it is preferred that the hybridization be performed under stringent hybridization conditions. An example of stringent conditions comprises incubating at 42° C. in a solution comprising 50% formamide, 5×SSC, and 1% SDS and washing at 65° C. in a solution comprising 0.2×SSC and 0.1% SDS.

An exemplary method for detecting the presence or absence of a biomarker protein or nucleic acid in a biological sample involves obtaining a biological sample (e.g. a tumor-associated body fluid) from a test subject and contacting the biological sample with a compound or an agent capable of detecting the polypeptide or nucleic acid (e.g., mRNA, genomic DNA, or cDNA). The detection methods of the invention can thus be used to detect mRNA, protein, cDNA, or genomic DNA, for example, in a biological sample in vitro as well as in vivo. For example, in vitro techniques for detection of mRNA include Northern hybridizations and in situ hybridizations. In vitro techniques for detection of a biomarker protein include ELISAs, IHC, Western blots, immunoprecipitations and immunofluorescence. In vivo techniques for detection of mRNA include PCR, Northern hybridizations and in situ hybridizations. Furthermore, in vivo techniques for detection of a biomarker protein include introducing into a subject a labeled antibody directed against the protein or fragment thereof. For example, the antibody can be labeled with a radioactive marker whose presence and location in a subject can be detected by standard imaging techniques.

In an alternative embodiment of this invention E-cadherin expression can be assessed by determining the degree of methylation of the promoter of the E-cadherin gene (CDH1), which is inversely proportional to expression from the gene, and thus can be used as a surrogate assay to estimate expression. Readily detectable methylation of the promoter (e.g. a strong signal during detection of a methylation-specific PCR-amplified nucleic acid product derived from a promoter methylation site) will be found when E-cadherin expression is low, whereas no detectable or low methylation of the promoter (e.g. no, or a comparatively weak, signal during detection of a methylation-specific PCR-amplified nucleic acid product derived from a promoter methylation site) corresponds to the situation where E-cadherin expression levels are high.

A general principle of diagnostic and prognostic assays as described herein involves preparing a sample or reaction mixture that may contain a biomarker, and a probe, under appropriate conditions and for a time sufficient to allow the biomarker and probe to interact and bind, thus forming a complex that can be removed and/or detected in the reaction mixture or sample. These assays can be conducted in a variety of ways.

For example, one method to conduct such an assay would involve anchoring the biomarker or probe onto a solid phase support, also referred to as a substrate, and detecting target biomarker/probe complexes anchored on the solid phase at the end of the reaction. In one embodiment of such a method, a sample from a subject, which is to be assayed for presence and/or concentration of biomarker, can be anchored onto a carrier or solid phase support. In another embodiment, the reverse situation is possible, in which the probe can be anchored to a solid phase and a sample from a subject can be allowed to react as an unanchored component of the assay.

There are many established methods for anchoring assay components to a solid phase. These include, without limitation, biomarker or probe molecules which are immobilized through conjugation of biotin and streptavidin. Such biotinylated assay components can be prepared from biotin-NHS(N-hydroxy-succinimide) using techniques known in the art (e.g., biotinylation kit, Pierce Chemicals, Rockford, Ill.), and immobilized in the wells of streptavidin-coated 96 well plates (Pierce Chemical). In certain embodiments, the surfaces with immobilized assay components can be prepared in advance and stored.

Other suitable carriers or solid phase supports for such assays include any material capable of binding the class of molecule to which the biomarker or probe belongs. Well-known supports or carriers include, but are not limited to, glass, polystyrene, nylon, polypropylene, nylon, polyethylene, dextran, amylases, natural and modified celluloses, polyacrylamides, gabbros, and magnetite.

In order to conduct assays with the above mentioned approaches, the non-immobilized component is added to the solid phase upon which the second component is anchored. After the reaction is complete, uncomplexed components may be removed (e.g., by washing) under conditions such that any complexes formed will remain immobilized upon the solid phase. The detection of biomarker/probe complexes anchored to the solid phase can be accomplished in a number of methods outlined herein.

In one embodiment, the probe, when it is the unanchored assay component, can be labeled for the purpose of detection and readout of the assay, either directly or indirectly, with detectable labels discussed herein and which are well-known to one skilled in the art.

It is also possible to directly detect biomarker/probe complex formation without further manipulation or labeling of either component (biomarker or probe), for example by utilizing the technique of fluorescence energy transfer (i.e. FET, see for example, Lakowicz et al., U.S. Pat. No. 5,631,169; Stavrianopoulos, et al., U.S. Pat. No. 4,868,103). A fluorophore label on the first, ‘donor’ molecule is selected such that, upon excitation with incident light of appropriate wavelength, its emitted fluorescent energy will be absorbed by a fluorescent label on a second ‘acceptor’ molecule, which in turn is able to fluoresce due to the absorbed energy. Alternately, the ‘donor’ protein molecule may simply utilize the natural fluorescent energy of tryptophan residues. Labels are chosen that emit different wavelengths of light, such that the ‘acceptor’ molecule label may be differentiated from that of the ‘donor’. Since the efficiency of energy transfer between the labels is related to the distance separating the molecules, spatial relationships between the molecules can be assessed. In a situation in which binding occurs between the molecules, the fluorescent emission of the ‘acceptor’ molecule label in the assay should be maximal. An FET binding event can be conveniently measured through standard fluorometric detection means well known in the art (e.g., using a fluorimeter).

In another embodiment, determination of the ability of a probe to recognize a biomarker can be accomplished without labeling either assay component (probe or biomarker) by utilizing a technology such as real-time Biomolecular Interaction Analysis (BIA) (see, e.g., Sjolander, S, and Urbaniczky, C., 1991, Anal. Chem. 63:2338-2345 and Szabo et al., 1995, Curr. Opin. Struct. Biol. 5:699-705). As used herein, “BIA” or “surface plasmon resonance” is a technology for studying biospecific interactions in real time, without labeling any of the interactants (e.g., BIAcore). Changes in the mass at the binding surface (indicative of a binding event) result in alterations of the refractive index of light near the surface (the optical phenomenon of surface plasmon resonance (SPR)), resulting in a detectable signal which can be used as an indication of real-time reactions between biological molecules.

Alternatively, in another embodiment, analogous diagnostic and prognostic assays can be conducted with biomarker and probe as solutes in a liquid phase. In such an assay, the complexed biomarker and probe are separated from uncomplexed components by any of a number of standard techniques, including but not limited to: differential centrifugation, chromatography, electrophoresis and immunoprecipitation. In differential centrifugation, biomarker/probe complexes may be separated from uncomplexed assay components through a series of centrifugal steps, due to the different sedimentation equilibria of complexes based on their different sizes and densities (see, for example, Rivas, G., and Minton, A. P., 1993, Trends Biochem Sci. 18(8):284-7). Standard chromatographic techniques may also be utilized to separate complexed molecules from uncomplexed ones. For example, gel filtration chromatography separates molecules based on size, and through the utilization of an appropriate gel filtration resin in a column format, for example, the relatively larger complex may be separated from the relatively smaller uncomplexed components. Similarly, the relatively different charge properties of the biomarker/probe complex as compared to the uncomplexed components may be exploited to differentiate the complex from uncomplexed components, for example through the utilization of ion-exchange chromatography resins. Such resins and chromatographic techniques are well known to one skilled in the art (see, e.g., Heegaard, N. H., 1998, J. Mol. Recognit. Winter 11(1-6):141-8; Hage, D. S., and Tweed, S. A. J. Chromatogr B Biomed Sci Appl 1997 Oct. 10; 699(1-2):499-525). Gel electrophoresis may also be employed to separate complexed assay components from unbound components (see, e.g., Ausubel et al., ed., Current Protocols in Molecular Biology, John Wiley & Sons, New York, 1987-1999). In this technique, protein or nucleic acid complexes are separated based on size or charge, for example. In order to maintain the binding interaction during the electrophoretic process, non-denaturing gel matrix materials and conditions in the absence of reducing agent are typically preferred. Appropriate conditions to the particular assay and components thereof will be well known to one skilled in the art.

In a particular embodiment, the level of biomarker mRNA can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding a biomarker of the present invention. Other suitable probes for use in the diagnostic assays of the invention are described herein. Hybridization of an mRNA with the probe indicates that the biomarker in question is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in an AFFYMETRIX® gene chip array. A skilled artisan can readily adapt known mRNA detection methods for use in detecting the level of mRNA encoded by the biomarkers of the present invention.

An alternative method for determining the level of mRNA biomarker in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample is prepared/processed using known histological methods. The sample is then immobilized on a support, typically a glass slide, and then contacted with a probe that can hybridize to mRNA that encodes the biomarker.

As an alternative to making determinations based on the absolute expression level of the biomarker, determinations may be based on the normalized expression level of the biomarker. Expression levels are normalized by correcting the absolute expression level of a biomarker by comparing its expression to the expression of a gene that is not a biomarker, e.g., a housekeeping gene that is constitutively expressed. Suitable genes for normalization include housekeeping genes, such as the actin gene. This normalization allows the comparison of the expression level in one sample, e.g., a patient sample, to another sample from a different patient, or from the same patient at a different time, or from a non-tumor sample, or between different tumor samples from the same patient.

Alternatively, the expression level can be provided as a relative expression level. To determine a relative expression level of a biomarker in patient samples, the level of expression of the biomarker is determined for about 10 or more samples of high biomarker expression versus low biomarker expression tumor cell samples, preferably 50 or more samples, prior to the determination of the expression level for the sample in question. The mean expression level of the biomarker assayed in the larger number of samples is determined and this is used as a baseline expression level for the biomarker. The expression level of the biomarker determined for the test sample (absolute level of expression) is then divided by the mean expression value obtained for that biomarker from high biomarker expression or low biomarker expression tumor cell samples. This provides a relative expression level.

In another embodiment of the present invention, a biomarker protein is detected. A preferred agent for detecting biomarker protein of the invention is an antibody capable of binding to such a protein or a fragment thereof, preferably an antibody with a detectable label. Antibodies can be polyclonal, or more preferably, monoclonal. An intact antibody, or a fragment or derivative thereof (e.g., Fab or F(ab′).sub.2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

Proteins from tumor cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether tumor cells express a biomarker of the present invention.

In one format, antibodies, or antibody fragments or derivatives, can be used in methods such as Western blots or immunofluorescence techniques to detect the expressed proteins. In such uses, it is generally preferable to immobilize either the antibody or proteins on a solid support. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite.

One skilled in the art will know many other suitable carriers for binding antibody or antigen, and will be able to adapt such support for use with the present invention. For example, protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies. Exemplary non-immune pairs are biotin-streptavidin, intrinsic factor-vitamin B₁₂, folic acid-folate binding protein and the like.

A variety of methods are available to covalently label antibodies with members of specific binding pairs. Methods are selected based upon the nature of the member of the specific binding pair, the type of linkage desired, and the tolerance of the antibody to various conjugation chemistries. Biotin can be covalently coupled to antibodies by utilizing commercially available active derivatives. Some of these are biotin-N-hydroxy-succinimide which binds to amine groups on proteins; biotin hydrazide which binds to carbohydrate moieties, aldehydes and carboxyl groups via a carbodiimide coupling; and biotin maleimide and iodoacetyl biotin which bind to sulfhydryl groups. Fluorescein can be coupled to protein amine groups using fluorescein isothiocyanate. Dinitrophenyl groups can be coupled to protein amine groups using 2,4-dinitrobenzene sulfate or 2,4-dinitrofluorobenzene. Other standard methods of conjugation can be employed to couple monoclonal antibodies to a member of a specific binding pair including dialdehyde, carbodiimide coupling, homofunctional crosslinking, and heterobifunctional crosslinking Carbodiimide coupling is an effective method of coupling carboxyl groups on one substance to amine groups on another. Carbodiimide coupling is facilitated by using the commercially available reagent 1-ethyl-3-(dimethyl-aminopropyl)-carbodiimide (EDAC).

Homobifunctional crosslinkers, including the bifunctional imidoesters and bifunctional N-hydroxysuccinimide esters, are commercially available and are employed for coupling amine groups on one substance to amine groups on another. Heterobifunctional crosslinkers are reagents which possess different functional groups. The most common commercially available heterobifunctional crosslinkers have an amine reactive N-hydroxysuccinimide ester as one functional group, and a sulfhydryl reactive group as the second functional group. The most common sulfhydryl reactive groups are maleimides, pyridyl disulfides and active halogens. One of the functional groups can be a photoactive aryl nitrene, which upon irradiation reacts with a variety of groups.

The detectably-labeled antibody or detectably-labeled member of the specific binding pair is prepared by coupling to a reporter, which can be a radioactive isotope, enzyme, fluorogenic, chemiluminescent or electrochemical materials. Two commonly used radioactive isotopes are ¹²⁵I and ³H. Standard radioactive isotopic labeling procedures include the chloramine T, lactoperoxidase and Bolton-Hunter methods for ¹²⁵I and reductive methylation for ³H. The term “detectably-labeled” refers to a molecule labeled in such a way that it can be readily detected by the intrinsic enzymic activity of the label or by the binding to the label of another component, which can itself be readily detected.

Enzymes suitable for use in this invention include, but are not limited to, horseradish peroxidase, alkaline phosphatase, β-galactosidase, glucose oxidase, luciferases, including firefly and renilla, β-lactamase, urease, green fluorescent protein (GFP) and lysozyme. Enzyme labeling is facilitated by using dialdehyde, carbodiimide coupling, homobifunctional crosslinkers and heterobifunctional crosslinkers as described above for coupling an antibody with a member of a specific binding pair.

The labeling method chosen depends on the functional groups available on the enzyme and the material to be labeled, and the tolerance of both to the conjugation conditions. The labeling method used in the present invention can be one of, but not limited to, any conventional methods currently employed including those described by Engvall and Pearlmann, Immunochemistry 8, 871 (1971), Avrameas and Ternynck, Immunochemistry 8, 1175 (1975), Ishikawa et al., J. Immunoassay 4(3):209-327 (1983) and Jablonski, Anal. Biochem. 148:199 (1985).

Labeling can be accomplished by indirect methods such as using spacers or other members of specific binding pairs. An example of this is the detection of a biotinylated antibody with unlabeled streptavidin and biotinylated enzyme, with streptavidin and biotinylated enzyme being added either sequentially or simultaneously. Thus, according to the present invention, the antibody used to detect can be detectably-labeled directly with a reporter or indirectly with a first member of a specific binding pair. When the antibody is coupled to a first member of a specific binding pair, then detection is effected by reacting the antibody-first member of a specific binding complex with the second member of the binding pair that is labeled or unlabeled as mentioned above.

Moreover, the unlabeled detector antibody can be detected by reacting the unlabeled antibody with a labeled antibody specific for the unlabeled antibody. In this instance “detectably-labeled” as used above is taken to mean containing an epitope by which an antibody specific for the unlabeled antibody can bind. Such an anti-antibody can be labeled directly or indirectly using any of the approaches discussed above. For example, the anti-antibody can be coupled to biotin which is detected by reacting with the streptavidin-horseradish peroxidase system discussed above.

In one embodiment of this invention biotin is utilized. The biotinylated antibody is in turn reacted with streptavidin-horseradish peroxidase complex. Orthophenylenediamine, 4-chloro-naphthol, tetramethylbenzidine (TMB), ABTS, BTS or ASA can be used to effect chromogenic detection.

In one immunoassay format for practicing this invention, a forward sandwich assay is used in which the capture reagent has been immobilized, using conventional techniques, on the surface of a support. Suitable supports used in assays include synthetic polymer supports, such as polypropylene, polystyrene, substituted polystyrene, e.g. aminated or carboxylated polystyrene, polyacrylamides, polyamides, polyvinylchloride, glass beads, agarose, or nitrocellulose.

The invention also encompasses kits for detecting the presence of a biomarker protein or nucleic acid in a biological sample. Such kits can be used to determine if a subject is suffering from or is at increased risk of developing a tumor that is less susceptible to inhibition by EGFR kinase inhibitors. For example, the kit can comprise a labeled compound or agent capable of detecting a biomarker protein or nucleic acid in a biological sample and means for determining the amount of the protein or mRNA in the sample (e.g., an antibody which binds the protein or a fragment thereof, or an oligonucleotide probe which binds to DNA or mRNA encoding the protein). Kits can also include instructions for interpreting the results obtained using the kit.

For antibody-based kits, the kit can comprise, for example: (1) a first antibody (e.g., attached to a solid support) which binds to a biomarker protein; and, optionally, (2) a second, different antibody which binds to either the protein or the first antibody and is conjugated to a detectable label.

For oligonucleotide-based kits, the kit can comprise, for example: (1) an oligonucleotide, e.g., a detectably labeled oligonucleotide, which hybridizes to a nucleic acid sequence encoding a biomarker protein or (2) a pair of primers useful for amplifying a biomarker nucleic acid molecule. The kit can also comprise, e.g., a buffering agent, a preservative, or a protein stabilizing agent. The kit can further comprise components necessary for detecting the detectable label (e.g., an enzyme or a substrate). The kit can also contain a control sample or a series of control samples which can be assayed and compared to the test sample. Each component of the kit can be enclosed within an individual container and all of the various containers can be within a single package, along with instructions for interpreting the results of the assays performed using the kit.

The present invention further provides the methods for treating tumors or tumor metastases in a patient with cancer as described herein, comprising administering to the patient a therapeutically effective amount of an EGFR kinase inhibitor and in addition, simultaneously or sequentially, one or more other cytotoxic, chemotherapeutic or anti-cancer agents, or compounds that enhance the effects of such agents. In the context of this invention, other anti-cancer agents includes, for example, other cytotoxic, chemotherapeutic or anti-cancer agents, or compounds that enhance the effects of such agents, anti-hormonal agents, angiogenesis inhibitors, agents that inhibit or reverse EMT (e.g. TGF-beta receptor inhibitors), tumor cell pro-apoptotic or apoptosis-stimulating agents, histone deacetylase (HDAC) inhibitors, histone demethylase inhibitors, DNA methyltransferase inhibitors, signal transduction inhibitors, anti-proliferative agents, anti-HER2 antibody or an immunotherapeutically active fragment thereof, anti-proliferative agents, COX II (cyclooxygenase II) inhibitors, and agents capable of enhancing antitumor immune responses.

In the context of this invention, additional other cytotoxic, chemotherapeutic or anti-cancer agents, or compounds that enhance the effects of such agents, include, for example: alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6MP), 6-thiocguanine (6TG), cytarabine (Ara-C), 5-fluorouracil (5-FU), capecitabine (e.g. XELODA®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

The present invention further provides the preceding methods for treating tumors or tumor metastases in a patient, comprising administering to the patient a therapeutically effective amount of an EGFR kinase inhibitor and in addition, simultaneously or sequentially, one or more anti-hormonal agents. As used herein, the term “anti-hormonal agent” includes natural or synthetic organic or peptidic compounds that act to regulate or inhibit hormone action on tumors.

Antihormonal agents include, for example: steroid receptor antagonists, anti-estrogens such as tamoxifen, raloxifene, aromatase inhibiting 4(5)-imidazoles, other aromatase inhibitors, 42-hydroxytamoxifen, trioxifene, keoxifene, LY 117018, onapristone, and toremifene (e.g. FARESTON®); anti-androgens such as flutamide, nilutamide, bicalutamide, leuprolide, and goserelin; and pharmaceutically acceptable salts, acids or derivatives of any of the above; agonists and/or antagonists of glycoprotein hormones such as follicle stimulating hormone (FSH), thyroid stimulating hormone (TSH), and luteinizing hormone (LH) and LHRH (leuteinizing hormone-releasing hormone); the LHRH agonist goserelin acetate, commercially available as ZOLADEX® (AstraZeneca); the LHRH antagonist D-alaninamide N-acetyl-3-(2-naphthalenyl)-D-alanyl-4-chloro-D-phenylalanyl-3-(3-pyridinyl)-D-alanyl-L-seryl-N-6-(3-pyridinylcarbonyl)-L-lysyl-N-6-(3-pyridinylcarbonyl)-D-lysyl-L-leucyl-N-6-(1-methylethyl)-L-lysyl-L-proline (e.g ANTIDE®, Ares-Serono); the LHRH antagonist ganirelix acetate; the steroidal anti-androgens cyproterone acetate (CPA) and megestrol acetate, commercially available as MEGACE® (Bristol-Myers Oncology); the nonsteroidal anti-androgen flutamide (2-methyl-N-[4,20-nitro-3-(trifluoromethyl)phenylpropanamide), commercially available as EULEXIN® (Schering Corp.); the non-steroidal anti-androgen nilutamide, (5,5-dimethyl-3-[4-nitro-3-(trifluoromethyl-4′-nitrophenyl)-4,4-dimethyl-imidazolidine-dione); and antagonists for other non-permissive receptors, such as antagonists for RAR, RXR, TR, VDR, and the like.

The use of the cytotoxic and other anti-cancer agents described above in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

The present invention further provides the preceding methods for treating tumors or tumor metastases in a patient, comprising administering to the patient a therapeutically effective amount of an EGFR kinase inhibitor and in addition, simultaneously or sequentially, one or more angiogenesis inhibitors.

Anti-angiogenic agents include, for example: VEGFR inhibitors, such as SU-5416 and SU-6668 (Sugen Inc. of South San Francisco, Calif., USA), or as described in, for example International Application Nos. WO 99/24440, WO 99/62890, WO 95/21613, WO 99/61422, WO 98/50356, WO 99/10349, WO 97/32856, WO 97/22596, WO 98/54093, WO 98/02438, WO 99/16755, and WO 98/02437, and U.S. Pat. Nos. 5,883,113, 5,886,020, 5,792,783, 5,834,504 and 6,235,764; VEGF inhibitors such as IM862 (Cytran Inc. of Kirkland, Wash., USA); angiozyme, a synthetic ribozyme from Ribozyme (Boulder, Colo.) and Chiron (Emeryville, Calif.); and antibodies to VEGF, such as bevacizumab (e.g. AVASTIN™, Genentech, South San Francisco, Calif.), a recombinant humanized antibody to VEGF; integrin receptor antagonists and integrin antagonists, such as to αᵥβ₃, αᵥβ₅ and αᵥβ₆ integrins, and subtypes thereof, e.g. cilengitide (EMD 121974), or the anti-integrin antibodies, such as for example αᵥβ₃ specific humanized antibodies (e.g. VITAXIN®); factors such as IFN-alpha (U.S. Pat. Nos. 41,530,901, 4,503,035, and 5,231,176); angiostatin and plasminogen fragments (e.g. kringle 1-4, kringle 5, kringle 1-3 (O'Reilly, M. S. et al. (1994) Cell 79:315-328; Cao et al. (1996) J. Biol. Chem. 271: 29461-29467; Cao et al. (1997) J. Biol. Chem. 272:22924-22928); endostatin (O'Reilly, M. S. et al. (1997) Cell 88:277; and International Patent Publication No. WO 97/15666); thrombospondin (TSP-1; Frazier, (1991) Curr. Opin. Cell Biol. 3:792); platelet factor 4 (PF4); plasminogen activator/urokinase inhibitors; urokinase receptor antagonists; heparinases; fumagillin analogs such as TNP-4701; suramin and suramin analogs; angiostatic steroids; bFGF antagonists; flk-1 and flt-1 antagonists; anti-angiogenesis agents such as MMP-2 (matrix-metalloproteinase 2) inhibitors and MMP-9 (matrix-metalloproteinase 9) inhibitors. Examples of useful matrix metalloproteinase inhibitors are described in International Patent Publication Nos. WO 96/33172, WO 96/27583, WO 98/07697, WO 98/03516, WO 98/34918, WO 98/34915, WO 98/33768, WO 98/30566, WO 90/05719, WO 99/52910, WO 99/52889, WO 99/29667, and WO 99/07675, European Patent Publication Nos. 818,442, 780,386, 1,004,578, 606,046, and 931,788; Great Britain Patent Publication No. 9912961, and U.S. Pat. Nos. 5,863,949 and 5,861,510. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

The present invention further provides the preceding methods for treating tumors or tumor metastases in a patient, comprising administering to the patient a therapeutically effective amount of an EGFR kinase inhibitor and in addition, simultaneously or sequentially, one or more tumor cell pro-apoptotic or apoptosis-stimulating agents.

The present invention further provides the preceding methods for treating tumors or tumor metastases in a patient, comprising administering to the patient a therapeutically effective amount of an EGFR kinase inhibitor and in addition, simultaneously or sequentially, one or more histone deacetylase (HDAC) inhibitors.

HDAC inhibitors include, for example: SB939, CHR-3996, CRA-024781, ITF2357, JNJ-26854165, JNJ-26481585 (Ortho Biotech), Vorinostat (suberoylanilide hydroxamic acid, SAHA; Merck), FK-228 (depsipeptide/FR-901228, Fujisawa, Osaka, Japan), Phenylbutyrate (Elan Pharmaceuticals, Dublin), LAQ824 and LBH589 (Novartis), PXD101 (TopoTarget, Copenhagen), MS-275 (Schering AG), Pyroxamide (Aton Pharma, Tarrytown, N.Y.), MGCD0103 (MethylGene, Montreal), NBM-HD-1 (NatureWise Biotech & Medicals Corporation), CI-994 (Pfizer Inc), Pivanex (Titan Pharmaceuticals Inc), Romidepsin (Gloucester Pharmaceuticals), and Entinostat (SNDX-275; Syndax Pharmaceuticals),

The present invention further provides the preceding methods for treating tumors or tumor metastases in a patient, comprising administering to the patient a therapeutically effective amount of an EGFR kinase inhibitor and in addition, simultaneously or sequentially, one or more histone demethylase inhibitors.

The present invention further provides the preceding methods for treating tumors or tumor metastases in a patient, comprising administering to the patient a therapeutically effective amount of an EGFR kinase inhibitor and in addition, simultaneously or sequentially, one or more DNA methyltransferase inhibitors. DNA methyltransferase inhibitors include, for example: S-110 (Supergen, Dublin, Calif.), Zebularine, Procaine, (−) epigallocatechin-3-gallate (EGCG), and Psammaplins.

The present invention further provides the preceding methods for treating tumors or tumor metastases in a patient, comprising administering to the patient a therapeutically effective amount of an EGFR kinase inhibitor and in addition, simultaneously or sequentially, one or more signal transduction inhibitors.

Signal transduction inhibitors include, for example: erbB2 receptor inhibitors, such as organic molecules, or antibodies that bind to the erbB2 receptor, for example, trastuzumab (e.g. HERCEPTIN®); inhibitors of other protein tyrosine-kinases, e.g. imitinib (e.g. GLEEVEC®); ras inhibitors; raf inhibitors; MEK inhibitors; PAK1 and PAK2 kinase inhibitors; mTOR inhibitors, such as, for example, rapamycin and its analogues (e.g. CCI-779, RAD001 and AP23573), including mTOR inhibitors that bind to and directly inhibits both mTORC1 and mTORC2 kinases; mTOR inhibitors that are dual PI3K/mTOR kinase inhibitors, such as for example the compound PI-103 as described in Fan, Q-W et al (2006) Cancer Cell 9:341-349 and Knight, Z. A. et al. (2006) Cell 125:733-747; mTOR inhibitors that are dual inhibitors of mTOR kinase and one or more other PIKK (or PIK-related) kinase family members. Such members include MEC1, TEL1, RAD3, MEI-41, DNA-PK, ATM, ATR, TRRAP, PI3K, and PI4K kinases; cyclin dependent kinase inhibitors; protein kinase C inhibitors; PI-3 kinase inhibitors; and PDK-1 inhibitors (see Dancey, J. and Sausville, E. A. (2003) Nature Rev. Drug Discovery 2:92-313, for a description of several examples of such inhibitors, and their use in clinical trials for the treatment of cancer).

ErbB2 receptor inhibitors include, for example: ErbB2 receptor inhibitors, such as GW-282974 (Glaxo Wellcome plc), monoclonal antibodies such as AR-209 (Aronex Pharmaceuticals Inc. of The Woodlands, Tex., USA) and 2B-1 (Chiron), and erbB2 inhibitors such as those described in International Publication Nos. WO 98/02434, WO 99/35146, WO 99/35132, WO 98/02437, WO 97/13760, and WO 95/19970, and U.S. Pat. Nos. 5,587,458, 5,877,305, 6,465,449 and 6,541,481.

As used herein, an mTOR inhibitor includes any mTOR inhibitor that is currently known in the art, and includes any chemical entity that, upon administration to a patient, results in inhibition of mTOR in the patient. An mTOR inhibitor can inhibit mTOR by any biochemical mechanism, including competition at the ATP binding site, competition elsewhere at the catalytic site of mTOR kinase, non-competitive inhibition, irreversible inhibition (e.g. covalent protein modification), or modulation of the interactions of other protein subunits or binding proteins with mTOR kinase in a way that results in inhibition of mTOR kinase activity (e.g. modulation of the interaction of mTOR with FKBP12, GβL, (mLST8), RAPTOR (mKOG1), or RICTOR (mAVO3)). Specific examples of mTOR inhibitors include: rapamycin; other rapamycin macrolides, or rapamycin analogues, derivatives or prodrugs; RAD001 (also known as Everolimus, RAD001 is an alkylated rapamycin (40-O-(2-hydroxyethyl)-rapamycin), disclosed in U.S. Pat. No. 5,665,772; Novartis); CCI-779 (also known as Temsirolimus, CCI-779 is an ester of rapamycin (42-ester with 3-hydroxy-2-hydroxymethyl-2-methylpropionic acid), disclosed in U.S. Pat. No. 5,362,718; Wyeth); AP23573 or AP23841 (Ariad Pharmaceuticals); ABT-578 (40-epi-(tetrazolyl)-rapamycin; Abbott Laboratories); KU-0059475 (Kudus Pharmaceuticals); and TAFA-93 (a rapamycin prodrug; Isotechnika) Examples of rapamycin analogs and derivatives known in the art include those compounds described in U.S. Pat. Nos. 6,329,386; 6,200,985; 6,117,863; 6,015,815; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; and 5,023,262; all of which are incorporated herein by reference. Rapamycin derivatives are also disclosed for example in WO 94/09010, WO 95/16691, WO 96/41807, or WO 99/15530, which are incorporated herein by reference. Such analogs and derivatives include 32-deoxorapamycin, 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxyethyl)-rapamycin, 40-O-(2-hydroxyethyl)-rapamycin, 32-deoxorapamycin and 16-pent-2-ynyloxy-32(S)-dihydro-rapamycin. Rapamycin derivatives may also include the so-called rapalogs, e.g. as disclosed in WO 98/02441 and WO01/14387 (e.g. AP23573, AP23464, AP23675 or AP23841). Further examples of a rapamycin derivative are those disclosed under the name biolimus-7 or biolimus-9 (BIOLIMUS A9™) (Biosensors International, Singapore). Any of the above rapamycin analogs or derivatives may be readily prepared by procedures as described in the above references.

As used herein, the term “mTOR inhibitor that binds to and directly inhibits both mTORC1 and mTORC2 kinases” refers to any mTOR inhibitor that binds to and directly inhibits both mTORC1 and mTORC2 kinases, and includes any chemical entity that, upon administration to a patient, binds to and results in direct inhibition of both mTORC1 and mTORC2 kinases in the patient. Examples of mTOR inhibitors useful in the invention described herein include those disclosed and claimed in U.S. patent application Ser. No. 11/599,663, filed Nov. 15, 2006, a series of compounds that inhibit mTOR by binding to and directly inhibiting both mTORC1 and mTORC2 kinases.

The present invention further provides the preceding methods for treating tumors or tumor metastases in a patient, comprising administering to the patient a therapeutically effective amount of an EGFR kinase inhibitor and in addition, simultaneously or sequentially, an anti-HER2 antibody or an immunotherapeutically active fragment thereof.

The present invention further provides the preceding methods for treating tumors or tumor metastases in a patient, comprising administering to the patient a therapeutically effective amount of an EGFR kinase inhibitor and in addition, simultaneously or sequentially, one or more additional anti-proliferative agents.

Additional antiproliferative agents include, for example: Inhibitors of the enzyme farnesyl protein transferase, platelet-derived growth factor receptor (PDGFR) kinase inhibitors, including the compounds disclosed and claimed in U.S. Pat. Nos. 6,080,769, 6,194,438, 6,258,824, 6,586,447, 6,071,935, 6,495,564, 6,150,377, 6,596,735 and 6,479,513, and International Patent Publication WO 01/40217. Antiproliferative agents also include IGF-1R kinase inhibitors and fibroblast growth factor receptor (FGFR) kinase inhibitors.

As used herein, the term “PDGFR kinase inhibitor” includes any PDGFR kinase inhibitor that is currently known in the art, and includes any chemical entity that, upon administration to a patient, results in inhibition of a biological activity associated with activation of the PDGFR in the patient, including any of the downstream biological effects otherwise resulting from the binding to PDGFR of its natural ligand. Such PDGFR kinase inhibitors include any agent that can block PDGFR activation or any of the downstream biological effects of PDGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the PDGFR, thereby making the receptor inaccessible to its natural ligand so that its normal biological activity is prevented or reduced. Alternatively, such an inhibitor can act by modulating the dimerization of PDGFR polypeptides, or interaction of PDGFR polypeptide with other proteins, or enhance ubiquitination and endocytotic degradation of PDGFR. PDGFR kinase inhibitors include but are not limited to small molecule inhibitors, antibodies or antibody fragments, antisense constructs, small inhibitory RNAs (i.e. RNA interference by dsRNA; RNAi), and ribozymes. PDGFR kinase inhibitors include anti-PDGF (anti-platelet-derived growth factor) or anti-PDGFR aptamers, anti-PDGF or anti-PDGFR antibodies, or soluble PDGF receptor decoys that prevent binding of a PDGF to its cognate receptor. In a preferred embodiment, the PDGFR kinase inhibitor is a small organic molecule or an antibody that binds specifically to the human PDGFR. The ability of a compound or agent to serve as a PDGFR kinase inhibitor may be determined according to the methods known in art and, further, as set forth in, e.g., Dai et al., (2001) Genes & Dev. 15: 1913-25; Zippel, et al., (1989) Eur. J. Cell Biol. 50(2):428-34; and Zwiller, et al., (1991) Oncogene 6: 219-21.

The invention includes PDGFR kinase inhibitors known in the art as well as those supported below and any and all equivalents that are within the scope of ordinary skill to create. For example, inhibitory antibodies directed against PDGF are known in the art, e.g., those described in U.S. Pat. Nos. 5,976,534, 5,833,986, 5,817,310, 5,882,644, 5,662,904, 5,620,687, 5,468,468, and PCT WO 2003/025019, the contents of which are incorporated by reference in their entirety. In addition, the invention includes N-phenyl-2-pyrimidine-amine derivatives that are PDGFR kinase inhibitors, such as those disclosed in U.S. Pat. No. 5,521,184, as well as WO2003/013541, WO2003/078404, WO2003/099771, WO2003/015282, and WO2004/05282 which are hereby incorporated in their entirety by reference.

Small molecules that block the action of PDGF are known in the art, e.g., those described in U.S. patent or Published Application U.S. Pat. No. 6,528,526 (PDGFR tyrosine kinase inhibitors), U.S. Pat. No. 6,524,347 (PDGFR tyrosine kinase inhibitors), U.S. Pat. No. 6,482,834 (PDGFR tyrosine kinase inhibitors), U.S. Pat. No. 6,472,391 (PDGFR tyrosine kinase inhibitors), U.S. Pat. Nos. 6,949,563, 6,696,434, 6,331,555, 6,251,905, 6,245,760, 6,207,667, 5,990,141, 5,700,822, 5,618,837, 5,731,326, and 2005/0154014, and International Published Application Nos. WO 2005/021531, WO 2005/021544, and WO 2005/021537, the contents of which are incorporated by reference in their entirety.

Proteins and polypeptides that block the action of PDGF are known in the art, e.g., those described in U.S. Pat. No. 6,350,731 (PDGF peptide analogs), U.S. Pat. No. 5,952,304, the contents of which are incorporated by reference in their entirety.

Bis mono- and bicyclic aryl and heteroaryl compounds which inhibit EGF and/or PDGF receptor tyrosine kinase are known in the art, e.g., those described in, e.g. U.S. Pat. Nos. 5,476,851, 5,480,883, 5,656,643, 5,795,889, and 6,057,320, the contents of which are incorporated by reference in their entirety.

Antisense oligonucleotides for the inhibition of PDGF are known in the art, e.g., those described in U.S. Pat. Nos. 5,869,462, and 5,821,234, the contents of each of which are incorporated by reference in their entirety.

Aptamers (also known as nucleic acid ligands) for the inhibition of PDGF are known in the art, e.g., those described in, e.g., U.S. Pat. Nos. 6,582,918, 6,229,002, 6,207,816, 5,668,264, 5,674,685, and 5,723,594, the contents of each of which are incorporated by reference in their entirety.

Other compounds for inhibiting PDGF known in the art include those described in U.S. Pat. Nos. 5,238,950, 5,418,135, 5,674,892, 5,693,610, 5,700,822, 5,700,823, 5,728,726, 5,795,910, 5,817,310, 5,872,218, 5,932,580, 5,932,602, 5,958,959, 5,990,141, 6,358,954, 6,537,988 and 6,673,798, the contents of each of which are incorporated by reference in their entirety.

A number of types of tyrosine kinase inhibitors that are selective for tyrosine kinase receptor enzymes such as PDGFR are known (see, e.g., Spada and Myers ((1995) Exp. Opin. Ther. Patents, 5: 805) and Bridges ((1995) Exp. Opin. Ther. Patents, 5: 1245). Additionally Law and Lydon have summarized the anti-cancer potential of tyrosine kinase inhibitors ((1996) Emerging Drugs: The Prospect For Improved Medicines, 241-260). For example, U.S. Pat. No. 6,528,526 describes substituted quinoxaline compounds that selectively inhibit platelet-derived growth factor-receptor (PDGFR) tyrosine kinase activity. The known inhibitors of PDGFR tyrosine kinase activity includes quinoline-based inhibitors reported by Maguire et al., ((1994) J. Med. Chem., 37: 2129), and by Dolle, et al., ((1994) J. Med. Chem., 37: 2627). A class of phenylamino-pyrimidine-based inhibitors was recently reported by Traxler, et al., in EP 564409 and by Zimmerman et al., ((1996) Biorg. Med. Chem. Lett., 6: 1221-1226) and by Buchdunger, et al., ((1995) Proc. Nat. Acad. Sci. (USA), 92: 2558). Quinazoline derivatives that are useful in inhibiting PDGF receptor tyrosine kinase activity include bismono- and bicyclic aryl compounds and heteroaryl compounds (see, e.g., WO 92/20642), quinoxaline derivatives (see (1994) Cancer Res., 54: 6106-6114), pyrimidine derivatives (Japanese Published Patent Application No. 87834/94) and dimethoxyquinoline derivatives (see Abstracts of the 116th Annual Meeting of the Pharmaceutical Society of Japan (Kanazawa), (1996), 2, p. 275, 29(C2) 15-2).

Specific preferred examples of small molecule PDGFR kinase inhibitors that can be used according to the present invention include Imatinib (GLEEVEC®; Novartis); SU-12248 (sunitib malate, SUTENT®; Pfizer); Dasatinib (SPRYCEL®; BMS; also known as BMS-354825); Sorafenib (NEXAVAR®; Bayer; also known as Bay-43-9006); AG-13736 (Axitinib; Pfizer); RPR127963 (Sanofi-Aventis); CP-868596 (Pfizer/OSI Pharmaceuticals); MLN-518 (tandutinib; Millennium Pharmaceuticals); AMG-706 (Motesanib; Amgen); ARAVA® (leflunomide; Sanofi-Aventis; also known as SU101), and OSI-930 (OSI Pharmaceuticals); Additional preferred examples of small molecule PDGFR kinase inhibitors that are also FGFR kinase inhibitors that can be used according to the present invention include XL-999 (Exelixis); SU6668 (Pfizer); CHIR-258/TKI-258 (Chiron); R04383596 (Hoffmann-La Roche) and BIBF-1120 (Boehringer Ingelheim).

As used herein, the term “FGFR kinase inhibitor” includes any FGFR kinase inhibitor that is currently known in the art, and includes any chemical entity that, upon administration to a patient, results in inhibition of a biological activity associated with activation of FGFR in the patient, including any of the downstream biological effects otherwise resulting from the binding to FGFR of its natural ligand. Such FGFR kinase inhibitors include any agent that can block FGFR activation or any of the downstream biological effects of FGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the FGF receptor, thereby making the receptor inaccessible to its natural ligand so that its normal biological activity is prevented or reduced. Alternatively, such an inhibitor can act by modulating the dimerization of FGFR polypeptides, or interaction of FGFR polypeptide with other proteins, or enhance ubiquitination and endocytotic degradation of FGFR. FGFR kinase inhibitors include but are not limited to small molecule inhibitors, antibodies or antibody fragments, antisense constructs, small inhibitory RNAs (i.e. RNA interference by dsRNA; RNAi), and ribozymes. FGFR kinase inhibitors include anti-FGF (anti-fibroblast growth factor) or anti-FGFR aptamers, anti-FGF or anti-FGFR antibodies, or soluble FGFR receptor decoys that prevent binding of a FGFR to its cognate receptor. In a preferred embodiment, the FGFR kinase inhibitor is a small organic molecule or an antibody that binds specifically to the human FGFR. Anti-FGFR antibodies include FR1-H7 (FGFR-1) and FR3-D11 (FGFR-3) (Imclone Systems, Inc.).

FGFR kinase inhibitors also include compounds that inhibit FGFR signal transduction by affecting the ability of heparan sulfate proteoglycans to modulate FGFR activity. Heparan sulfate proteoglycans in the extracellular matrix can mediate the actions of FGF, e.g., protection from proteolysis, localization, storage, and internalization of growth factors (Faham, S. et al. (1998) Curr. Opin. Struct. Biol., 8:578-586), and may serve as low affinity FGF receptors that act to present FGF to its cognate FGFR, and/or to facilitate receptor oligomerization (Galzie, Z. et al. (1997) Biochem. Cell. Biol., 75:669-685).

The invention includes FGFR kinase inhibitors known in the art (e.g. PD173074) as well as those supported below and any and all equivalents that are within the scope of ordinary skill to create.

Examples of chemicals that may antagonize fibroblast growth factor (FGF) action, and can thus be used as FGFR kinase inhibitors in the methods described herein, include suramin, structural analogs of suramin, pentosan polysulfate, scopolamine, angiostatin, sprouty, estradiol, carboxymethylbenzylamine dextran (CMDB7), suradista, insulin-like growth factor binding protein-3, ethanol, heparin (e.g., 6-O-desulfated heparin), small molecule heparin, protamine sulfate, cyclosporin A, or RNA ligands for bFGF.

Other agents or compounds for inhibiting FGFR kinase known in the art include those described in U.S. Pat. No. 7,151,176 (Bristol-Myers Squibb Company; Pyrrolotriazine compounds); U.S. Pat. No. 7,102,002 (Bristol-Myers Squibb Company; pyrrolotriazine compounds); U.S. Pat. No. 5,132,408 (Salk Institute; peptide FGF antagonists); and U.S. Pat. No. 5,945,422 (Warner-Lambert Company; 2-amino-substituted pyrido[2,3-d]pyrimidines); U.S. published Patent application Nos. 2005/0256154 (4-amino-thieno[3,2-c]pyridine-7-carboxylic acid amide compounds); and 2004/0204427 (pyrimidino compounds); and published International Patent Applications WO-2007019884 (Merck Patent GmbH; N-(3-pyrazolyl)-N′-4-(4-pyridinyloxy)phenyl)urea compounds); WO-2007009773 (Novartis AG; pyrazolo[1,5-a]pyrimidin-7-yl amine derivatives); WO-2007014123 (Five Prime Therapeutics, Inc.; FGFR fusion proteins); WO-2006134989 (Kyowa Hakko Kogyo Co., Ltd.; nitrogenous heterocycle compounds); WO-2006112479 (Kyowa Hakko Kogyo Co., Ltd.; azaheterocycles); WO-2006108482 (Merck Patent GmbH; 9-(4-ureidophenyl)purine compounds); WO-2006105844 (Merck Patent GmbH; N-(3-pyrazolyl)-N′-4-(4-pyridinyloxy)phenyl)urea compounds); WO-2006094600 (Merck Patent GmbH; tetrahydropyrroloquinoline derivatives); WO-2006050800 (Merck Patent GmbH; N,N′-diarylurea derivatives); WO-2006050779 (Merck Patent GmbH; N,N′-diarylurea derivatives); WO-2006042599 (Merck Patent GmbH; phenylurea derivatives); WO-2005066211 (Five Prime Therapeutics, Inc.; anti-FGFR antibodies); WO-2005054246 (Merck Patent GmbH; heterocyclyl amines); WO-2005028448 (Merck Patent GmbH; 2-amino-1-benzyl-substituted benzimidazole derivatives); WO-2005011597 (Irm Llc; substituted heterocyclic derivatives); WO-2004093812 (Irm Llc/Scripps; 6-phenyl-7H-pyrrolo[2,3-d]pyrimidine derivatives); WO-2004046152 (F. Hoffmann La Roche AG; pyrimido[4,5-e]oxadiazine derivatives); WO-2004041822 (F. Hoffmann La Roche AG; pyrimido[4,5-d]pyrimidine derivatives); WO-2004018472 (F. Hoffmann La Roche AG; pyrimido[4,5-d]pyrimidine derivatives); WO-2004013145 (Bristol-Myers Squibb Company; pyrrolotriazine derivatives); WO-2004009784 (Bristol-Myers Squibb Company; pyrrolo[2,1-f][1,2,4]triazin-6-yl compounds); WO-2004009601 (Bristol-Myers Squibb Company; azaindole compounds); WO-2004001059 (Bristol-Myers Squibb Company; heterocyclic derivatives); WO-02102972 (Prochon Biotech Ltd./Morphosys AG; anti-FGFR antibodies); WO-02102973 (Prochon Biotech Ltd.;
anti-FGFR antibodies); WO-00212238 (Warner-Lambert Company; 2-(pyridin-4-ylamino)-6-dialkoxyphenyl-pyrido[2,3-d]pyrimidin-7-one derivatives); WO-00170977 (Amgen, Inc.; FGFR-L and derivatives); WO-00132653 (Cephalon, Inc.; pyrazolone derivatives); WO-00046380 (Chiron Corporation; FGFR-Ig fusion proteins); and WO-00015781 (Eli Lilly; polypeptides related to the human SPROUTY-1 protein).

Specific preferred examples of small molecule FGFR kinase inhibitors that can be used according to the present invention include RO-4396686 (Hoffmann-La Roche); CHIR-258 (Chiron; also known as TKI-258); PD 173074 (Pfizer); PD 166866 (Pfizer); ENK-834 and ENK-835 (both Enkam Pharmaceuticals A/S); and SU5402 (Pfizer). Additional preferred examples of small molecule FGFR kinase inhibitors that are also PDGFR kinase inhibitors that can be used according to the present invention include XL-999 (Exelixis); SU6668 (Pfizer); CHIR-258/TKI-258 (Chiron); R04383596 (Hoffmann-La Roche), and BIBF-1120 (Boehringer Ingelheim).

As used herein, the term “IGF-1R kinase inhibitor” includes any IGF-1R kinase inhibitor that is currently known in the art, and includes any chemical entity that, upon administration to a patient, results in inhibition of a biological activity associated with activation of the IGF-1 receptor in the patient, including any of the downstream biological effects otherwise resulting from the binding to IGF-1R of its natural ligand. Such IGF-1R kinase inhibitors include any agent that can block IGF-1R activation or any of the downstream biological effects of IGF-1R activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the IGF-1 receptor, thereby making the receptor inaccessible to its natural ligand so that its normal biological activity is prevented or reduced. Alternatively, such an inhibitor can act by modulating the dimerization of IGF-1R polypeptides, or interaction of IGF-1R polypeptide with other proteins, or enhance ubiquitination and endocytotic degradation of IGF-1R. An IGF-1R kinase inhibitor can also act by reducing the amount of IGF-1 available to activate IGF-1R, by for example antagonizing the binding of IGF-1 to its receptor, by reducing the level of IGF-1, or by promoting the association of IGF-1 with proteins other than IGF-1R such as IGF binding proteins (e.g. IGFBP3). IGF-1R kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the IGF-1R kinase inhibitor is a small organic molecule or an antibody that binds specifically to the human IGF-1R.

IGF-1R kinase inhibitors include, for example imidazopyrazine IGF-1R kinase inhibitors, azabicyclic amine inhibitors, quinazoline IGF-1R kinase inhibitors, pyrido-pyrimidine IGF-1R kinase inhibitors, pyrimido-pyrimidine IGF-1R kinase inhibitors, pyrrolo-pyrimidine IGF-1R kinase inhibitors, pyrazolo-pyrimidine IGF-1R kinase inhibitors, phenylamino-pyrimidine IGF-1R kinase inhibitors, oxindole IGF-1R kinase inhibitors, indolocarbazole IGF-1R kinase inhibitors, phthalazine IGF-1R kinase inhibitors, isoflavone IGF-1R kinase inhibitors, quinalone IGF-1R kinase inhibitors, and tyrphostin IGF-1R kinase inhibitors, and all pharmaceutically acceptable salts and solvates of such IGF-1R kinase inhibitors.

Examples of IGF-1R kinase inhibitors include those in International Patent Publication No. WO 05/097800, that describes azabicyclic amine derivatives, International Patent Publication No. WO 05/037836, that describes imidazopyrazine IGF-1R kinase inhibitors, International Patent Publication Nos. WO 03/018021 and WO 03/018022, that describe pyrimidines for treating IGF-1R related disorders, International Patent Publication Nos. WO 02/102804 and WO 02/102805, that describe cyclolignans and cyclolignans as IGF-1R inhibitors, International Patent Publication No. WO 02/092599, that describes pyrrolopyrimidines for the treatment of a disease which responds to an inhibition of the IGF-1R tyrosine kinase, International Patent Publication No. WO 01/72751, that describes pyrrolopyrimidines as tyrosine kinase inhibitors, and in International Patent Publication No. WO 00/71129, that describes pyrrolotriazine inhibitors of kinases, and in International Patent Publication No. WO 97/28161, that describes pyrrolo[2,3-d]pyrimidines and their use as tyrosine kinase inhibitors, Parrizas, et al., which describes tyrphostins with in vitro and in vivo IGF-1R inhibitory activity (Endocrinology, 138:1427-1433 (1997)), International Patent Publication No. WO 00/35455, that describes heteroaryl-aryl ureas as IGF-1R inhibitors, International Patent Publication No. WO 03/048133, that describes pyrimidine derivatives as modulators of IGF-1R, International Patent Publication No. WO 03/024967, WO 03/035614, WO 03/035615, WO 03/035616, and WO 03/035619, that describe chemical compounds with inhibitory effects towards kinase proteins, International Patent Publication No. WO 03/068265, that describes methods and compositions for treating hyperproliferative conditions, International Patent Publication No. WO 00/17203, that describes pyrrolopyrimidines as protein kinase inhibitors, Japanese Patent Publication No. JP 07/133,280, that describes a cephem compound, its production and antimicrobial composition, Albert, A. et al., Journal of the Chemical Society, 11: 1540-1547 (1970), which describes pteridine studies and pteridines unsubstituted in the 4-position, and A. Albert et al., Chem. Biol. Pteridines Proc. Int. Symp., 4th, 4: 1-5 (1969) which describes a synthesis of pteridines (unsubstituted in the 4-position) from pyrazines, via 3-4-dihydropteridines.

Additional, specific examples of IGF-1R kinase inhibitors that can be used according to the present invention include h7C10 (Centre de Recherche Pierre Fabre), an IGF-1 antagonist; EM-164 (ImmunoGen Inc.), an IGF-1R modulator; CP-751871 (Pfizer Inc.), an IGF-1 antagonist; lanreotide (Ipsen), an IGF-1 antagonist; IGF-1R oligonucleotides (Lynx Therapeutics Inc.); IGF-1 oligonucleotides (National Cancer Institute); IGF-1R protein-tyrosine kinase inhibitors in development by Novartis (e.g. NVP-AEW541, Garcia-Echeverria, C. et al. (2004) Cancer Cell 5:231-239; or NVP-ADW742, Mitsiades, C. S. et al. (2004) Cancer Cell 5:221-230); IGF-1R protein-tyrosine kinase inhibitors (Ontogen Corp); OSI-906 (OSI Pharmaceuticals); AG-1024 (Camirand, A. et al. (2005) Breast Cancer Research 7:R570-R579 (DOI 10.1186/bcr1028); Camirand, A. and Pollak, M. (2004) Brit. J. Cancer 90:1825-1829; Pfizer Inc.), an IGF-1 antagonist; the tyrphostins AG-538 and I-OMe-AG 538; BMS-536924, a small molecule inhibitor of IGF-1R; PNU-145156E (Pharmacia & Upjohn SpA), an IGF-1 antagonist; BMS 536924, a dual IGF-1R and IR kinase inhibitor (Bristol-Myers Squibb); AEW541 (Novartis); GSK621659A (Glaxo Smith-Kline); INSM-18 (Insmed); and XL-228 (Exelixis).

Antibody-based IGF-1R kinase inhibitors include any anti-IGF-1R antibody or antibody fragment that can partially or completely block IGF-1R activation by its natural ligand. Antibody-based IGF-1R kinase inhibitors also include any anti-IGF-1 antibody or antibody fragment that can partially or completely block IGF-1R activation. Non-limiting examples of antibody-based IGF-1R kinase inhibitors include those described in Larsson, O. et al (2005) Brit. J. Cancer 92:2097-2101 and Ibrahim, Y. H. and Yee, D. (2005) Clin. Cancer Res. 11:944s-950s; or being developed by Imclone (e.g. IMC-A12), or AMG-479, an anti-IGF-1R antibody (Amgen); R1507, an anti-IGF-1R antibody (Genmab/Roche); AVE-1642, an anti-IGF-1R antibody (Immunogen/Sanofi-Aventis); MK 0646 or h7C10, an anti-IGF-1R antibody (Merck); or antibodies being develop by Schering-Plough Research Institute (e.g. SCH 717454 or 19D12; or as described in US Patent Application Publication Nos. US 2005/0136063 A1 and US 2004/0018191 A1). The IGF-1R kinase inhibitor can be a monoclonal antibody, or an antibody or antibody fragment having the binding specificity thereof.

The present invention further provides the preceding methods for treating tumors or tumor metastases in a patient, comprising administering to the patient a therapeutically effective amount of an EGFR kinase inhibitor and in addition, simultaneously or sequentially, a COX II (cyclooxygenase II) inhibitor. Examples of useful COX-II inhibitors include alecoxib (e.g. CELEBREX™), valdecoxib, and rofecoxib.

The present invention further provides the preceding methods for treating tumors or tumor metastases in a patient, comprising administering to the patient a therapeutically effective amount of an EGFR kinase inhibitor and in addition, simultaneously or sequentially, treatment with radiation or a radiopharmaceutical.

The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Where the EGFR kinase inhibitor according to this invention is an antibody, it is also possible to label the antibody with such radioactive isotopes.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anti-cancer agents. Parameters of adjuvant radiation therapies are, for example, contained in International Patent Publication WO 99/60023.

The present invention further provides the preceding methods for treating tumors or tumor metastases in a patient, comprising administering to the patient a therapeutically effective amount of an EGFR kinase inhibitor and in addition, simultaneously or sequentially, treatment with one or more agents capable of enhancing antitumor immune responses.

Agents capable of enhancing antitumor immune responses include, for example: CTLA4 (cytotoxic lymphocyte antigen 4) antibodies (e.g. MDX-CTLA4), and other agents capable of blocking CTLA4. Specific CTLA4 antibodies that can be used in the present invention include those described in U.S. Pat. No. 6,682,736.

In the context of this invention, an “effective amount” of an agent or therapy is as defined above. A “sub-therapeutic amount” of an agent or therapy is an amount less than the effective amount for that agent or therapy, but when combined with an effective or sub-therapeutic amount of another agent or therapy can produce a result desired by the physician, due to, for example, synergy in the resulting efficacious effects, or reduced side effects.

As used herein, the term “patient” preferably refers to a human in need of treatment with an EGFR kinase inhibitor for cancer. However, the term “patient” can also refer to non-human animals, preferably mammals such as dogs, cats, horses, cows, pigs, sheep and non-human primates, among others, that are in need of treatment with an EGFR kinase inhibitor.

In a preferred embodiment, the patient is a human in need of treatment for cancer. The cancer of the patient is preferably any cancer treatable, either partially or completely, by administration of an EGFR kinase inhibitor. The cancer may be, for example, lung cancer, non-small cell lung cancer, bronchioloalviolar cell lung cancer, bone cancer, pancreatic cancer, skin cancer, cancer of the head and neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, colorectal cancer, cancer of the small intestine, cancer of the endocrine system, cancer of the thyroid gland, cancer of the parathyroid gland, cancer of the adrenal gland (e.g. adrenocortical carcinoma), sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers.

For purposes of the present invention, “co-administration of” and “co-administering” an EGFR kinase inhibitor with an additional anti-cancer agent (both components referred to hereinafter as the “two active agents”) refer to any administration of the two active agents, either separately or together, where the two active agents are administered as part of an appropriate dose regimen designed to obtain the benefit of the combination therapy. Thus, the two active agents can be administered either as part of the same pharmaceutical composition or in separate pharmaceutical compositions. The additional agent can be administered prior to, at the same time as, or subsequent to administration of the EGFR kinase inhibitor, or in some combination thereof. Where the EGFR kinase inhibitor is administered to the patient at repeated intervals, e.g., during a standard course of treatment, the additional agent can be administered prior to, at the same time as, or subsequent to, each administration of the EGFR kinase inhibitor, or some combination thereof, or at different intervals in relation to the EGFR kinase inhibitor treatment, or in a single dose prior to, at any time during, or subsequent to the course of treatment with the EGFR kinase inhibitor.

The EGFR kinase inhibitor will typically be administered to the patient in a dose regimen that provides for the most effective treatment of the cancer (from both efficacy and safety perspectives) for which the patient is being treated, as known in the art, and as disclosed, e.g. in International Patent Publication No. WO 01/34574. In conducting the treatment method of the present invention, the EGFR kinase inhibitor can be administered in any effective manner known in the art, such as by oral, topical, intravenous, intra-peritoneal, intramuscular, intra-articular, subcutaneous, intranasal, intra-ocular, vaginal, rectal, or intradermal routes, depending upon the type of cancer being treated, the type of EGFR kinase inhibitor being used (for example, small molecule, antibody, RNAi, ribozyme or antisense construct), and the medical judgement of the prescribing physician as based, e.g., on the results of published clinical studies.

The amount of EGFR kinase inhibitor administered and the timing of EGFR kinase inhibitor administration will depend on the type (species, gender, age, weight, etc.) and condition of the patient being treated, the severity of the disease or condition being treated, and on the route of administration. For example, small molecule EGFR kinase inhibitors can be administered to a patient in doses ranging from 0.001 to 100 mg/kg of body weight per day or per week in single or divided doses, or by continuous infusion (see for example, International Patent Publication No. WO 01/34574). In particular, erlotinib HCl can be administered to a patient in doses ranging from 5-200 mg per day, or 100-1600 mg per week, in single or divided doses, or by continuous infusion. A preferred dose is 150 mg/day. Antibody-based EGFR kinase inhibitors, or antisense, RNAi or ribozyme constructs, can be administered to a patient in doses ranging from 0.1 to 100 mg/kg of body weight per day or per week in single or divided doses, or by continuous infusion. In some instances, dosage levels below the lower limit of the aforesaid range may be more than adequate, while in other cases still larger doses may be employed without causing any harmful side effect, provided that such larger doses are first divided into several small doses for administration throughout the day.

The EGFR kinase inhibitors and other additional agents can be administered either separately or together by the same or different routes, and in a wide variety of different dosage forms. For example, the EGFR kinase inhibitor is preferably administered orally or parenterally. Where the EGFR kinase inhibitor is erlotinib HCl (TARCEVA®), oral administration is preferable. Both the EGFR kinase inhibitor and other additional agents can be administered in single or multiple doses.

The EGFR kinase inhibitor can be administered with various pharmaceutically acceptable inert carriers in the form of tablets, capsules, lozenges, troches, hard candies, powders, sprays, creams, salves, suppositories, jellies, gels, pastes, lotions, ointments, elixirs, syrups, and the like. Administration of such dosage forms can be carried out in single or multiple doses. Carriers include solid diluents or fillers, sterile aqueous media and various non-toxic organic solvents, etc. Oral pharmaceutical compositions can be suitably sweetened and/or flavored.

The EGFR kinase inhibitor can be combined together with various pharmaceutically acceptable inert carriers in the form of sprays, creams, salves, suppositories, jellies, gels, pastes, lotions, ointments, and the like. Administration of such dosage forms can be carried out in single or multiple doses. Carriers include solid diluents or fillers, sterile aqueous media, and various non-toxic organic solvents, etc. All formulations comprising proteinaceous EGFR kinase inhibitors should be selected so as to avoid denaturation and/or degradation and loss of biological activity of the inhibitor.

Methods of preparing pharmaceutical compositions comprising an EGFR kinase inhibitor are known in the art, and are described, e.g. in International Patent Publication No. WO 01/34574. In view of the teaching of the present invention, methods of preparing pharmaceutical compositions comprising an EGFR kinase inhibitor will be apparent from the above-cited publications and from other known references, such as Remington's Pharmaceutical Sciences, Mack Publishing Company, Easton, Pa., 18^{th} edition (1990).

For oral administration of EGFR kinase inhibitors, tablets containing one or both of the active agents are combined with any of various excipients such as, for example, micro-crystalline cellulose, sodium citrate, calcium carbonate, dicalcium phosphate and glycine, along with various disintegrants such as starch (and preferably corn, potato or tapioca starch), alginic acid and certain complex silicates, together with granulation binders like polyvinyl pyrrolidone, sucrose, gelatin and acacia. Additionally, lubricating agents such as magnesium stearate, sodium lauryl sulfate and talc are often very useful for tableting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the EGFR kinase inhibitor may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration of either or both of the active agents, solutions in either sesame or peanut oil or in aqueous propylene glycol may be employed, as well as sterile aqueous solutions comprising the active agent or a corresponding water-soluble salt thereof. Such sterile aqueous solutions are preferably suitably buffered, and are also preferably rendered isotonic, e.g., with sufficient saline or glucose. These particular aqueous solutions are especially suitable for intravenous, intramuscular, subcutaneous and intraperitoneal injection purposes. The oily solutions are suitable for intra-articular, intramuscular and subcutaneous injection purposes. The preparation of all these solutions under sterile conditions is readily accomplished by standard pharmaceutical techniques well known to those skilled in the art. Any parenteral formulation selected for administration of proteinaceous EGFR kinase inhibitors should be selected so as to avoid denaturation and loss of biological activity of the inhibitor.

Additionally, it is possible to topically administer either or both of the active agents, by way of, for example, creams, lotions, jellies, gels, pastes, ointments, salves and the like, in accordance with standard pharmaceutical practice. For example, a topical formulation comprising an EGFR kinase inhibitor in about 0.1% (w/v) to about 5% (w/v) concentration can be prepared.

For veterinary purposes, the active agents can be administered separately or together to animals using any of the forms and by any of the routes described above.

In a preferred embodiment, the EGFR kinase inhibitor is administered in the form of a capsule, bolus, tablet, liquid drench, by injection or as an implant. As an alternative, the EGFR kinase inhibitor can be administered with the animal feedstuff, and for this purpose a concentrated feed additive or premix may be prepared for a normal animal feed. Such formulations are prepared in a conventional manner in accordance with standard veterinary practice.

As used herein, the term “EGFR kinase inhibitor” includes any EGFR kinase inhibitor that is currently known in the art, and includes any chemical entity that, upon administration to a patient, results in inhibition of a biological activity associated with activation of the EGFR in the patient, including any of the downstream biological effects otherwise resulting from the binding to EGFR of its natural ligand. Such EGFR kinase inhibitors include any agent that can block EGFR activation or any of the downstream biological effects of EGFR activation that are relevant to treating cancer in a patient. Such an inhibitor can act by binding directly to the intracellular domain of the receptor and inhibiting its kinase activity. Alternatively, such an inhibitor can act by occupying the ligand binding site or a portion thereof of the EGFR, thereby making the receptor inaccessible to its natural ligand so that its normal biological activity is prevented or reduced. Alternatively, such an inhibitor can act by modulating the dimerization of EGFR polypeptides, or interaction of EGFR polypeptide with other proteins, or enhance ubiquitination and endocytotic degradation of EGFR. EGFR kinase inhibitors include but are not limited to low molecular weight inhibitors, antibodies or antibody fragments, antisense constructs, small inhibitory RNAs (i.e. RNA interference by dsRNA; RNAi), and ribozymes. In a preferred embodiment, the EGFR kinase inhibitor is a small organic molecule or an antibody that binds specifically to the human EGFR.

EGFR kinase inhibitors that include, for example quinazoline EGFR kinase inhibitors, pyrido-pyrimidine EGFR kinase inhibitors, pyrimido-pyrimidine EGFR kinase inhibitors, pyrrolo-pyrimidine EGFR kinase inhibitors, pyrazolo-pyrimidine EGFR kinase inhibitors, phenylamino-pyrimidine EGFR kinase inhibitors, oxindole EGFR kinase inhibitors, indolocarbazole EGFR kinase inhibitors, phthalazine EGFR kinase inhibitors, isoflavone EGFR kinase inhibitors, quinalone EGFR kinase inhibitors, and tyrphostin EGFR kinase inhibitors, such as those described in the following patent publications, and all pharmaceutically acceptable salts and solvates of said EGFR kinase inhibitors: International Patent Publication Nos. WO 96/33980, WO 96/30347, WO 97/30034, WO 97/30044, WO 97/38994, WO 97/49688, WO 98/02434, WO 97/38983, WO 95/19774, WO 95/19970, WO 97/13771, WO 98/02437, WO 98/02438, WO 97/32881, WO 98/33798, WO 97/32880, WO 97/3288, WO 97/02266, WO 97/27199, WO 98/07726, WO 97/34895, WO 96/31510, WO 98/14449, WO 98/14450, WO 98/14451, WO 95/09847, WO 97/19065, WO 98/17662, WO 99/35146, WO 99/35132, WO 99/07701, and WO 92/20642; European Patent Application Nos. EP 520722, EP 566226, EP 787772, EP 837063, and EP 682027; U.S. Pat. Nos. 5,747,498, 5,789,427, 5,650,415, and 5,656,643; and German Patent Application No. DE 19629652. Additional non-limiting examples of low molecular weight EGFR kinase inhibitors include any of the EGFR kinase inhibitors described in Traxler, P., 1998, Exp. Opin. Ther. Patents 8(12):1599-1625.

Specific preferred examples of low molecular weight EGFR kinase inhibitors that can be used according to the present invention include [6,7-bis(2-methoxyethoxy)-4-quinazolin-4-yl]-(3-ethynylphenyl)amine (also known as OSI-774, erlotinib, or TARCEVA® (erlotinib HCl); OSI Pharmaceuticals/Genentech/Roche) (U.S. Pat. No. 5,747,498; International Patent Publication No. WO 01/34574, and Moyer, J. D. et al. (1997) Cancer Res. 57:4838-4848); canertinib (also known as CI-1033, and formerly known as PD183805; Pfizer) (Sherwood et al., 1999, Proc. Am. Assoc. Cancer Res. 40:723); PD-158780 (Pfizer); AG-1478 (University of California); CGP-59326 (Novartis); PKI-166 (Novartis); EKB-569 (Wyeth); GW-2016 (also known as GW-572016 or lapatinib ditosylate; GSK); vandetanib (ZD6474; Astrazeneca), PF00299804 (Pfizer), and gefitinib (also known as ZD1839 or IRESSA™; Astrazeneca) (Woodburn et al., 1997, Proc. Am. Assoc. Cancer Res. 38:633). A particularly preferred low molecular weight EGFR kinase inhibitor that can be used according to the present invention is [6,7-bis(2-methoxyethoxy)-4-quinazolin-4-yl]-(3-ethynylphenyl)amine (i.e. erlotinib), its hydrochloride salt (i.e. erlotinib HCl, TARCEVA®), or other salt forms (e.g. erlotinib mesylate).

Antibody-based EGFR kinase inhibitors include any anti-EGFR antibody or antibody fragment that can partially or completely block EGFR activation by its natural ligand. Non-limiting examples of antibody-based EGFR kinase inhibitors include those described in Modjtahedi, H., et al., 1993, Br. J. Cancer 67:247-253; Teramoto, T., et al., 1996, Cancer 77:639-645; Goldstein et al., 1995, Clin. Cancer Res. 1:1311-1318; Huang, S. M., et al., 1999, Cancer Res. 15:59(8):1935-40; and Yang, X., et al., 1999, Cancer Res. 59:1236-1243. Thus, the EGFR kinase inhibitor can be the monoclonal antibody Mab E7.6.3 (Yang, X. D. et al. (1999) Cancer Res. 59:1236-43), or Mab C225 (ATCC Accession No. HB-8508), or an antibody or antibody fragment having the binding specificity thereof. Suitable monoclonal antibody EGFR kinase inhibitors include, but are not limited to, IMC-C225 (also known as cetuximab or ERBITUX™; Imclone Systems), panitumumab (also known as ABX-EGF; Abgenix), matuzumab (also known as EMD 72000; Merck KgaA, Darmstadt), RH3 (York Medical Bioscience Inc.), MDX-447 (Medarex/Merck KgaA), nimotuzumab (h-R3), zalutumumab, and ch806 (targeting mutant EGFRvIII).

Additional antibody-based EGFR kinase inhibitors can be raised according to known methods by administering the appropriate antigen or epitope to a host animal selected, e.g., from pigs, cows, horses, rabbits, goats, sheep, and mice, among others. Various adjuvants known in the art can be used to enhance antibody production.

Although antibodies useful in practicing the invention can be polyclonal, monoclonal antibodies are preferred. Monoclonal antibodies against EGFR can be prepared and isolated using any technique that provides for the production of antibody molecules by continuous cell lines in culture. Techniques for production and isolation include but are not limited to the hybridoma technique originally described by Kohler and Milstein (Nature, 1975, 256: 495-497); the human B-cell hybridoma technique (Kosbor et al., 1983, Immunology Today 4:72; Cote et al., 1983, Proc. Natl. Acad. Sci. USA 80: 2026-2030); and the EBV-hybridoma technique (Cole et al, 1985, Monoclonal Antibodies and Cancer Therapy, Alan R. Liss, Inc., pp. 77-96).

Alternatively, techniques described for the production of single chain antibodies (see, e.g., U.S. Pat. No. 4,946,778) can be adapted to produce anti-EGFR single chain antibodies. Antibody-based EGFR kinase inhibitors useful in practicing the present invention also include anti-EGFR antibody fragments including but not limited to F(ab′).sub.2 fragments, which can be generated by pepsin digestion of an intact antibody molecule, and Fab fragments, which can be generated by reducing the disulfide bridges of the F(ab′).sub.2 fragments. Alternatively, Fab and/or scFv expression libraries can be constructed (see, e.g., Huse et al., 1989, Science 246: 1275-1281) to allow rapid identification of fragments having the desired specificity to EGFR.

Techniques for the production and isolation of monoclonal antibodies and antibody fragments are well-known in the art, and are described in Harlow and Lane, 1988, Antibodies: A Laboratory Manual, Cold Spring Harbor Laboratory, and in J. W. Goding, 1986, Monoclonal Antibodies: Principles and Practice, Academic Press, London. Humanized anti-EGFR antibodies and antibody fragments can also be prepared according to known techniques such as those described in Vaughn, T. J. et al., 1998, Nature Biotech. 16:535-539 and references cited therein, and such antibodies or fragments thereof are also useful in practicing the present invention.

EGFR kinase inhibitors for use in the present invention can alternatively be based on antisense oligonucleotide constructs. Anti-sense oligonucleotides, including anti-sense RNA molecules and anti-sense DNA molecules, would act to directly block the translation of EGFR mRNA by binding thereto and thus preventing protein translation or increasing mRNA degradation, thus decreasing the level of EGFR kinase protein, and thus activity, in a cell. For example, antisense oligonucleotides of at least about 15 bases and complementary to unique regions of the mRNA transcript sequence encoding EGFR can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as EGFR kinase inhibitors for use in the present invention. EGFR gene expression can be reduced by contacting the tumor, subject or cell with a small double stranded RNA (dsRNA), or a vector or construct causing the production of a small double stranded RNA, such that expression of EGFR is specifically inhibited (i.e. RNA interference or RNAi). Methods for selecting an appropriate dsRNA or dsRNA-encoding vector are well known in the art for genes whose sequence is known (e.g. see Tuschi, T., et al. (1999) Genes Dev. 13(24):3191-3197; Elbashir, S. M. et al. (2001) Nature 411:494-498; Hannon, G. J. (2002) Nature 418:244-251; McManus, M. T. and Sharp, P. A. (2002) Nature Reviews Genetics 3:737-747; Bremmelkamp, T. R. et al. (2002) Science 296:550-553; U.S. Pat. Nos. 6,573,099 and 6,506,559; and International Patent Publication Nos. WO 01/36646, WO 99/32619, and WO 01/68836).

Ribozymes can also function as EGFR kinase inhibitors for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of EGFR mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as EGFR kinase inhibitors can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

In the context of the methods of treatment of this invention, EGFR kinase inhibitors are used as a composition comprised of a pharmaceutically acceptable carrier and a non-toxic therapeutically effective amount of an EGFR kinase inhibitor compound (including pharmaceutically acceptable salts thereof).

The term “pharmaceutically acceptable salts” refers to salts prepared from pharmaceutically acceptable non-toxic bases or acids. When a compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (cupric and cuprous), ferric, ferrous, lithium, magnesium, manganese (manganic and manganous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When a compound used in the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Pharmaceutical compositions used in the present invention comprising an EGFR kinase inhibitor compound (including pharmaceutically acceptable salts thereof) as active ingredient, can include a pharmaceutically acceptable carrier and optionally other therapeutic ingredients or adjuvants. Other therapeutic agents may include those cytotoxic, chemotherapeutic or anti-cancer agents, or agents which enhance the effects of such agents, as listed above. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the EGFR kinase inhibitor compounds (including pharmaceutically acceptable salts thereof) of this invention can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, an EGFR kinase inhibitor compound (including pharmaceutically acceptable salts of each component thereof) may also be administered by controlled release means and/or delivery devices. The combination compositions may be prepared by any of the methods of pharmacy. In general, such methods include a step of bringing into association the active ingredients with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

An EGFR kinase inhibitor compound (including pharmaceutically acceptable salts thereof) used in this invention, can also be included in pharmaceutical compositions in combination with one or more other therapeutically active compounds. Other therapeutically active compounds may include those cytotoxic, chemotherapeutic or anti-cancer agents, or agents which enhance the effects of such agents, as listed above.

Thus in one embodiment of this invention, the pharmaceutical composition can comprise an EGFR kinase inhibitor compound in combination with an anti-cancer agent, wherein said anti-cancer agent is a member selected from the group consisting of alkylating drugs, antimetabolites, microtubule inhibitors, podophyllotoxins, antibiotics, nitrosoureas, hormone therapies, kinase inhibitors, activators of tumor cell apoptosis, and antiangiogenic agents.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition used for this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably contains from about 0.05 mg to about 5 g of the active ingredient.

For example, a formulation intended for the oral administration to humans may contain from about 0.5 mg to about 5 g of active agent, compounded with an appropriate and convenient amount of carrier material that may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions used in the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions used in the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Pharmaceutical compositions for the present invention can be in a form suitable for topical use such as, for example, an aerosol, cream, ointment, lotion, dusting powder, or the like. Further, the compositions can be in a form suitable for use in transdermal devices. These formulations may be prepared, utilizing an EGFR kinase inhibitor compound (including pharmaceutically acceptable salts thereof), via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions for this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing an EGFR kinase inhibitor compound (including pharmaceutically acceptable salts thereof) may also be prepared in powder or liquid concentrate form.

Dosage levels for the compounds used for practicing this invention will be approximately as described herein, or as described in the art for these compounds. It is understood, however, that the specific dose level for any particular patient will depend upon a variety of factors including the age, body weight, general health, sex, diet, time of administration, route of administration, rate of excretion, drug combination and the severity of the particular disease undergoing therapy.

Many alternative experimental methods known in the art may be successfully substituted for those specifically described herein in the practice of this invention, as for example described in many of the excellent manuals and textbooks available in the areas of technology relevant to this invention (e.g. Using Antibodies, A Laboratory Manual, edited by Harlow, E. and Lane, D., 1999, Cold Spring Harbor Laboratory Press, (e.g. ISBN 0-87969-544-7); Roe B. A. et. al. 1996, DNA Isolation and Sequencing (Essential Techniques Series), John Wiley & Sons. (e.g. ISBN 0-471-97324-0); Methods in Enzymology: Chimeric Genes and Proteins”, 2000, ed. J. Abelson, M. Simon, S. Emr, J. Thorner. Academic Press; Molecular Cloning: a Laboratory Manual, 2001, 3^{rd} Edition, by Joseph Sambrook and Peter MacCallum, (the former Maniatis Cloning manual) (e.g. ISBN 0-87969-577-3); Current Protocols in Molecular Biology, Ed. Fred M. Ausubel, et. al. John Wiley & Sons (e.g. ISBN 0-471-50338-X); Current Protocols in Protein Science, Ed. John E. Coligan, John Wiley & Sons (e.g. ISBN 0-471-11184-8); and Methods in Enzymology: Guide to protein Purification, 1990, Vol. 182, Ed. Deutscher, M. P., Acedemic Press, Inc. (e.g. ISBN 0-12-213585-7)), or as described in the many university and commercial websites devoted to describing experimental methods in molecular biology.

This invention will be better understood from the Experimental Details that follow. However, one skilled in the art will readily appreciate that the specific methods and results discussed are merely illustrative of the invention as described more fully in the claims which follow thereafter, and are not to be considered in any way limited thereto.

Experimental Details:

Introduction

The NCIC CTG. BR.21 study was a Phase 3 trial of TARCEVA® involving patients who had had progression after standard chemotherapy for non-small-cell lung cancer (i.e. 2^{nd} and 3^{rd} line NSCLC; Shepherd F A, et al. N Engl. J. Med 2005; 353:123-132.). Patients were randomly assigned in a 2:1 ratio to receive 150 mg of TARCEVA® (erlotinib HCl) daily or placebo. The primary end point was overall survival. Progression free survival and response were secondary end points. Separate written consent was obtained for optional tissue banking and correlative studies. The results, subsequently published, demonstrated a positive effect of TARCEVA® treatment on these outcomes and led to the approval of TARCEVA® for this indication.

Because the BR21 trial is one of the few TARCEVA® clinical trials that includes a large number of patients and an untreated placebo group, it is optimally designed to evaluate the predictive and prognostic potential of biomarkers. As such, the potential of vimentin and E-cadherin protein expression to serve as a predictive biomarker for overall survival and performance free survival was assessed using remaining tumor samples from the BR21 study. IHC analysis of tumor tissue samples received from NCIC was used to determine E-Cadherin and vimentin protein expression. Sensitivity analyses were performed to determine optimal cut points for E-Cadherin and vimentin staining for classification of E-Cadherin and vimentin as “High” or “Low” for use as prognostic and/or predictive markers for survival and progression-free survival. Further analyses were performed to evaluate possible correlations of biomarker status with clinical outcomes of survival, progression-free survival, response and disease control.

Materials and Methods

Tumor Biopsy, and Preparation of Slides for Immunohistochemistry.

Tumor tissue was obtained from previously cut slides. Standard histological processes were used in the production of slides for tissue acquisition. Once received from a site or archival facility, slides were kept at ambient room conditions prior to utilization.

Immunohistochemistry

The determination of the relative presence or absence of E-cadherin and or vimentin protein within tissues of selected tumors was determined by immunohistochemistry (IHC) on the formalin-fixed paraffin embedded tissue sections. Samples were treated initially with a retrieval method to maximize availability of epitopes. After treating the samples with anti-E-cadherin or anti-vimentin primary antibodies (biotinylated), excess antibody was removed by rinsing, and biomarkers visualized using the avidin-biotin peroxidase complex technique, with secondary and tertiary antibody steps to label the antibodies with HRP (horse radish peroxidase), and using DAB (3,3′-diaminobenzidine) as HRP substrate. Using light microscopy the localization and quantitation of the brown oxidized DAB product (chromagen), and thus E-cadherin or Vimentin, was assessed by a skilled pathologist.

Staining for E-cadherin and vimentin protein was accomplished on individual slides using commercially available kits specifically made for IHC detection of the selected proteins. Kit instructions were followed using histology laboratory Standard Operating Procedures and standard practices. The materials, procedures and protocols used (with a Dako Autostainer) were essentially as follows:

Retrieval Protocol

Materials:

This protocol is to be used with a steamer that reaches 94-95 degrees C.

And Target Retrieval Solution (10×) from Dako (S1699) pH 6, mixed 1:10 with deionized water

Protocol:

Deparaffanize using Hemo-De® and hydrate with deionized water.

Preheat Target Retrieval Solution (30 ml per jar) to 94-95 degrees C. (approximately 20-25 minutes).

Place slides in Coplin jars containing 30 ml of the Target solution (equal number of slides in each jar).

Bring temperature up to 94-95 degrees (approximately 10 minutes) let steam for 15 minutes.

Remove the top of steamer to the counter top and let cool for 20 minutes.

Rinse in deionized water and stain.

E-Cadherin Assay

Processed slides were stained using E-Cadherin antibody (clone 24E10; Cell Signaling, Danvers, Mass.: product number 3195). This antibody is a rabbit monoclonal IgG that can bind to either human or mouse E-cadherin.

Epitome retrieval was done using Target Retrieval Solution from Dako (Carpinteria, Calif.: product number S1699) for 10 minutes at 94-95 degrees C. followed by 20 minutes of cool done before proceeding with the detection system.

The primary antibody at a 1:50 dilution using antibody diluent from Dako (Carpinteria, Calif.: product number S0809) was incubated for 60 minutes.

The detection system is a rabbit Vectastain Elite ABC kit obtained from Vector Laboratories (Burlingame, Calif.: product number PK6106) used according to kit instructions. Visualization was done using DAB (diaminobenzidine), Dako (Carpinteria, Calif.: product number K3468). The slides were counter-stained with hematoxylin, dehydrated through graded alcohols and cleared through HEMO-DE® and cover-slipped.

Additional materials used were: Peroxidase blocking reagent, Dako (S2001), or alternatively a 3% hydrogen peroxide solution. PBS pH 7.4 plus Tween 20, Dako (S1966) 2.5 ml per 5 liters of PBS.

Protocol:

PBS rinse

H₂O₂ block: 10 minutes

Water rinse

PBS rinse

Protein block (from rabbit Kit): 20 minutes

No rinse, blow air

Primary antibody: 60 minutes

PBS rinse

Secondary antibody (from rabbit kit): 30 minutes

PBS rinse

Tertiary antibody (from rabbit kit): 30 minutes

PBS rinse

Switch (for hazardous product disposal)

DAB+: 10 minutes

Water rinse

Remove slides from stainer, lightly counter-stain, dehydrate through graded alcohols, clear and cover-slip.

Vimentin Assay

Processed slides were stained using vimentin antibody, (clone V9; Dako (Carpinteria, Calif.: product number M 0725). This antibody is a mouse monoclonal recognizing human vimentin but does not cross react with mouse.

Epitome retrieval was done using Target Retrieval solution from Dako (Carpinteria, Calif.: product number S1699) for 10 minutes at 94-95 degrees C. followed by 20 minutes of cool done before proceeding with the detection system.

The primary antibody at a 1:100 dilution using antibody diluent from Dako (Carpinteria, Calif.: product number S0809) is incubated for 30 minutes.

The detection system is a mouse Vectastain Elite ABC kit obtained from Vector Laboratories (Burlingame, Calif.: product number PK6102) use according to kit instructions. Visualization was done using DAB (diaminobenzidine), Dako (Carpinteria, Calif.: product number K3468). The slides were counter-stained with Gill's hematoxylin, dehydrated through graded alcohols and cleared through HEMO-DE® and cover-slipped.

Additional materials used were: Peroxidase blocking reagent, Dako (S2001), or alternatively a 3% hydrogen peroxide solution. PBS pH 7.4 plus Tween 20, Dako (S1966) 2.5 ml per 5 liters of PBS.

Protocol:

PBS rinse

H₂O₂ block: 10 minutes

Water rinse

PBS rinse

Protein block (from mouse Kit): 20 minutes

No rinse, blow air

Primary antibody: 30 minutes

PBS rinse

Secondary antibody (from mouse kit): 30 minutes

PBS rinse

Tertiary antibody (from mouse kit): 30 minutes

PBS rinse

Switch (for hazardous product disposal)

DAB+: 10 minutes

Water rinse

Remove slides from stainer, lightly counter-stain, dehydrate through graded alcohols, clear and cover-slip.

Quantitation of Immunostaining and Analysis

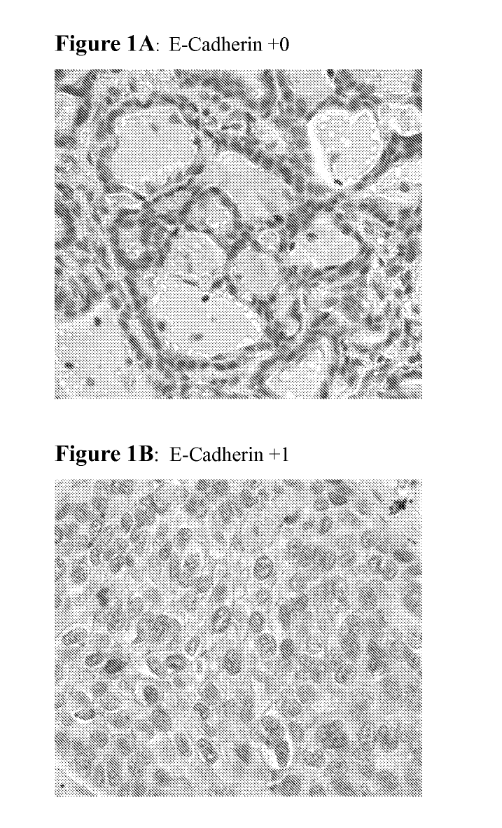

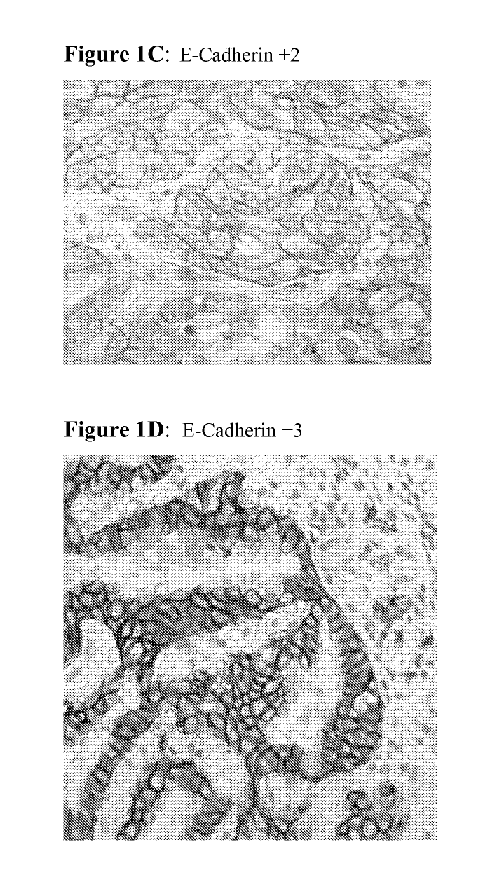

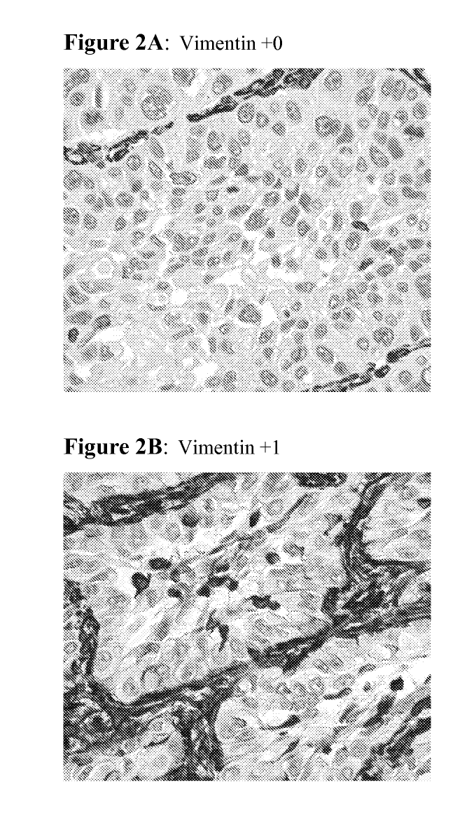

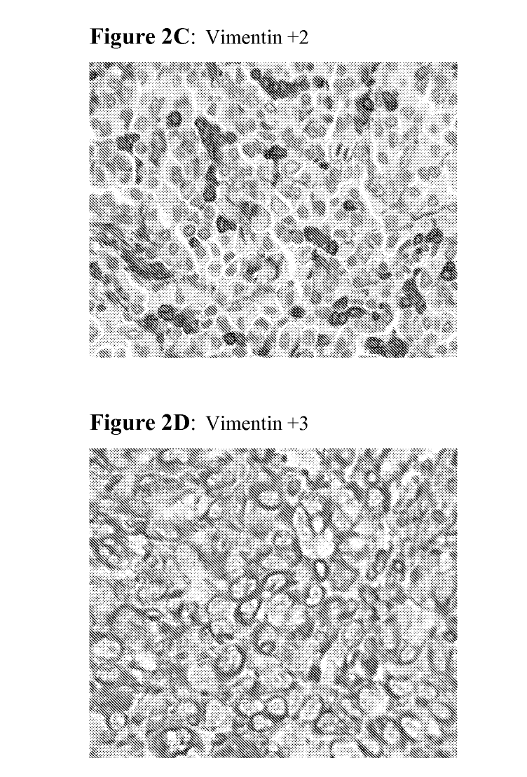

Stained slides were scored by an experienced pathologist. Slides were first evaluated for quality of tissue and quality of staining Acceptable slides were then evaluated, with the pathologist generating an ‘H-1-score’ based on a subjective interpretation of the staining intensity of the chromagen labeled antibody. Four intensity levels were used in scoring the stained sections: 0 for no staining, +1 for weak or minimal staining, +2 for moderate staining, and +3 for strong staining. The relative percentage of total target cells expressing an intensity level is recorded as data. From this data, the percentage of target cells expressing any intensity can be calculated and additional scoring paradigms can be calculated using the basic collected data. Representative examples of E-Cadherin and vimentin staining intensities are shown in FIGS. 1-2.

Treatment groups as randomized (ITT population) were analyzed using all patients with evaluable tissue. Disease progression as assessed by the investigator was used for PFS (Progression Free Survival) analysis. Potential predictive benefit of E-Cadherin or vimentin expression was assessed by comparing trends in the hazard ratios. No treatments by biomarker interaction tests were performed.
Three Scoring Methods were used for E-Cadherin and Vimentin:
1. % Any Staining of tumor cells
2. % of tumor cell Staining of Intensity +2 or +3
3. Composite Score
   Example: A sample read as 5% unstained, 35% staining of intensity +1, 45% staining of intensity +2, and 15% staining of intensity +3, would be scored as follows:
95% any staining
60% staining of intensity +2 or +3
0(5)+1(35)+2(45)+3(15)=Composite Score of 170
   Overall survival is defined as the time from the study treatment start date to the date of death. If the patient receives the study treatment but the patient is still alive or a death date is unavailable, overall survival is calculated as the difference between the study treatment start date and the last date the patient was known to be alive. These data are noted in the analyses as being “censored”. Progression free survival (PFS) is defined as the time from the study treatment start date to the documentation date of disease progression or the death date. As with overall survival, if no progression/death date is available, the PFS is calculated as the time from the study treatment start date to the last documented tumor assessment date, and is noted to be “censored”.

Results

TABLE 1
Summary of Tumor Tissue Samples Received and Evaluable Results:
Table T_1_Tissue_Samples
Summary of Tumor Tissue Samples Received and Evaluable Results

| Total | |
|---|---|
| Patients | |
| (N = 731) | |
| n | (%) |

| | | |
|---|---|---|
| Tissue Samples Received from NCIC | 163 | (22) |
| Patients with Known E-Cadherin Results | 95 | (13) |
| Patients with Known Vimentin Results | 95 | (13) |
| Patients with Known Results for either E-Cadherin or | 95 | (13) |
| Vimentin | | |
| Patients with Known Results for both E-Cadherin and | 95 | (13) |
| Vimentin | | |

Table 1 illustrates the number of patients that had tumor tissue samples, as well as the numbers of patients whose tissue samples passed QC requirements and yielded results for the E-Cadherin and vimentin assays by the OSI pathologist. Percentages reported are of the total number of patients on study (N=731).

TABLE 2
Success Rates for Tissue Analyses.
Table T_2_Tissue_Samples
Success Rates for Tissue Analysis

| | Total | |
|---|---|---|
| | Patients | |
| | with | |
| | Tissue | |
| | Samples | |
| | (N = 163) | |
| | n | (%) |
| Patients with Known E-Cadherin Results | 95 | (58) |
| Patients with Known Vimentin Results | 95 | (58) |
| Patients with Known Results for both E-Cadherin and | 95 | (58) |
| Vimentin | | |

Table 2 illustrates the number of patients whose tissue samples passed QC requirements and yielded results for the E-Cadherin and vimentin assays by the OSI pathologist. Percentages reported are of the number of patients who had tissue samples (N=163).

TABLE 3
Demographics.
Table T_3_Demographics
Demographics for Overall Population and Patients with Evaluable Results

| | | | Patients with | |
|---|---|---|---|---|
| | | | E-Cadherin | |
| | | | or | |
| | All | | Vimentin | |
| | Patients | | Results | |
| | (N = 731) | | (N = 95) | |
| Characteristics | n | (%) | n | (%) |
| Gender | | | | |
| Female | 256 | (35) | 36 | (38) |
| Male | 475 | (65) | 59 | (62) |
| Age (Years) | | | | |
| 18-39 | 11 | (2) | 0 | (0) |
| 40-64 | 441 | (60) | 61 | (64) |
| ≧65 | 279 | (38) | 34 | (36) |
| Race | | | | |
| White | 567 | (78) | 84 | (88) |
| Black | 30 | (4) | 6 | (6) |
| Native/Aboriginal | 1 | (<1) | 0 | (0) |
| Oriental | 91 | (12) | 5 | (5) |
| Indian Subcontinent | 1 | (<1) | 0 | (0) |
| Other | 41 | (6) | 0 | (0) |
| ECOG Performance Status | | | | |
| 0 | 98 | (13) | 13 | (14) |
| 1 | 388 | (53) | 53 | (56) |
| 2 | 182 | (25) | 18 | (19) |
| 3 | 63 | (9) | 11 | (12) |
| Weight Loss in Previous 6 Months | | | | |
| <5% | 486 | (66) | 61 | (64) |
| 5-10% | 132 | (18) | 16 | (17) |
| >10% | 81 | (11) | 15 | (16) |
| Unknown | 32 | (4) | 3 | (3) |
| Smoking History | | | | |
| Never smoked | 146 | (20) | 20 | (21) |
| Current or Ex-smoker | 545 | (75) | 68 | (72) |
| Unknown | 40 | (5) | 7 | (7) |

Table 3 describes the demographics for both the full study population (N=731) as well as for the subset of patients for whom E-Cadherin and vimentin results were available (N=95).

TABLE 4
Prior Therapy.
Table T_4_Prior_Therapy
Prior Therapies for Overall Population and Patients with Evaluable Results

| | | | Patients with | |
|---|---|---|---|---|
| | | | E-Cadherin | |
| | | | or | |
| | All | | Vimentin | |
| | Patients | | Results | |
| | (N = 731) | | (N = 95) | |
| | n | (%) | n | (%) |
| Previous Therapy | | | | |
| Chemotherapy | 731 | (100) | 95 | (100) |
| Surgery | 727 | (99) | 95 | (100) |
| Radiation | 407 | (56) | 48 | (51) |
| Hormonal Therapy | 2 | (<1) | 1 | (1) |
| Other Prior Therapy | 11 | (2) | 2 | (2) |
| Number of Prior Chemotherapy Regimens | | | | |
| 1 | 364 | (50) | 39 | (41) |
| 2 | 357 | (49) | 54 | (57) |
| 3 | 10 | (1) | 2 | (2) |
| Prior Platinum Therapy | | | | |
| No | 53 | (7) | 6 | (6) |
| Yes | 678 | (93) | 89 | (94) |
| Prior Taxane Therapy | | | | |
| No | 464 | (63) | 64 | (67) |
| Yes | 267 | (37) | 31 | (33) |

Table 4 describes the prior therapies received by the patients for both the full study population (N=731) as well as for the subset of patients for whom E-Cadherin and vimentin results were available (N=95).

TABLE 5
Disease Characteristics.
Table T_5_Disease_Characteristics
Disease Characteristics for Overall Population and Patients with Evaluable
Results

| | | | Patients with | |
|---|---|---|---|---|
| | | | E-Cadherin | |
| | | | or | |
| | All | | Vimentin | |
| | Patients | | Results | |
| | (N = 731) | | (N = 95) | |
| | n | (%) | n | (%) |
| Histological Classification | | | | |
| Adenocarcinoma | 365 | (50) | 49 | (52) |
| Squamous | 222 | (30) | 35 | (37) |
| Undifferentiated Large Cell | 64 | (9) | 9 | (6) |
| Mixed Non-Small Cell | 13 | (2) | 1 | (1) |
| Other | 67 | (9) | 4 | (4) |
| Stage of Disease at First Diagnosis | | | | |
| IA | 11 | (2) | 5 | (5) |
| IB | 23 | (3) | 7 | (7) |
| IIA | 10 | (1) | 1 | (1) |
| IIB | 22 | (3) | 6 | (6) |
| IIIA | 63 | (9) | 11 | (12) |
| IIIB | 273 | (37) | 23 | (24) |
| IV | 329 | (45) | 42 | (44) |
| Time From Initial Diagnosis to | | | | |
| Randomization (Months) | | | | |
| <6 | 97 | (13) | 8 | (8) |
| 6-12 | 242 | (33) | 30 | (32) |
| >12 | 392 | (54) | 57 | (60) |
| Time From the Most Recent Progression/ | | | | |
| Relapse to Randomization (Months) | | | | |
| <6 | 702 | (96) | 93 | (98) |
| 6-12 | 20 | (3) | 2 | (2) |
| >12 | 4 | (<1) | 0 | (0) |
| Missing | 5 | (<1) | 0 | (0) |

Table 5 describes the disease characteristics (histology, stage of disease, etc.) of the patients for both the full study population (N=731) as well as for the subset of patients for whom E-Cadherin and vimentin results were available (N=95).

TABLE 6
Evaluable Results.
Table T_6_Evaluable_Results
Summary of Evaluable E-Cadherin and Vimentin Results

| | Erlotinib | | Placebo | | All Patients | |
|---|---|---|---|---|---|---|
| | (N = 57) | | (N = 38) | | (N = 95) | |
| Biomarker | n | (%) | N | (%) | n | (%) |
| E-Cadherin | | | | | | |
| High | 38 | (67) | 22 | (58) | 60 | (63) |
| Low | 19 | (33) | 16 | (42) | 35 | (37) |
| Vimentin | | | | | | |
| Low | 41 | (72) | 26 | (68) | 67 | (71) |
| High | 16 | (28) | 12 | (32) | 28 | (29) |
| E-Cadherin/Vimentin | | | | | | |
| High/Low | 29 | (51) | 16 | (42) | 45 | (47) |
| High/High | 9 | (16) | 6 | (16) | 15 | (16) |
| Low/Low | 12 | (21) | 10 | (26) | 22 | (23) |
| Low/High | 7 | (12) | 6 | (16) | 13 | (14) |

Note:
E-Cadherin Status is High if at least 40% of the staining is intensity +2 or +3, Low if less than 40% of the staining is intensity +2 or +3.
Vimentin Status is Low if no more than 9% of the cells have any staining. If 10 or more percent are stained, vimentin Status is High.

Table 6 illustrates the numbers of patients in each treatment arm who were E-Cadherin high vs. low, and vimentin high vs. low for the subset of patients for whom E-Cadherin and vimentin results were available (N=95). P-values are determined from univariate Kaplan-Meier analyses. Hazard ratios with confidence limits are determined from Cox proportional hazards models.

TABLE 7
Overall Survival, Erlotinib vs. Placebo.
Table T_7_Survival
Overall Survival by E-Cadherin and Vimentin Results

| | Erlotinib | | Placebo | | | |
|---|---|---|---|---|---|---|
| | | Median | | Median | | Log- |
| | | Survival | | Survival | Hazard Ratio | Rank |
| Characteristics | N | Months | N | Months | (95% CI) | p-value |
| All Patients | 488 | 6.67 | 243 | 4.70 | 0.76 | 0.002 |
| | | | | | (0.64, 0.91) | |
| Patients with | 57 | 8.44 | 38 | 4.52 | 0.69 | 0.125 |
| Known | | | | | | |
| E-Cadherin | | | | | | |
| Results | | | | | | |
| | | | | | (0.43, 1.11) | |
| Patients with | 57 | 8.44 | 38 | 4.52 | 0.69 | 0.125 |
| Known | | | | | | |
| Vimentin | | | | | | |
| Results | | | | | | |
| | | | | | (0.43, 1.11) | |
| E-Cadherin | | | | | | |
| High | 38 | 11.3 | 22 | 4.23 | 0.47 | 0.015 |
| | | | | | (0.26, 0.88) | |
| Low | 19 | 4.86 | 16 | 6.83 | 1.12 | 0.769 |
| | | | | | (0.52, 2.44) | |
| Vimentin | | | | | | |
| Low | 41 | 6.11 | 26 | 5.42 | 0.99 | 0.965 |
| | | | | | (0.55, 1.76) | |
| High | 16 | 10.55 | 12 | 3.56 | 0.26 | 0.002 |
| | | | | | (0.11, 0.63) | |
| E-Cadherin/ | | | | | | |
| Vimentin | | | | | | |
| High/Low | 29 | 12.06 | 16 | 4.23 | 0.57 | 0.118 |
| | | | | | (0.28, 1.17) | |
| High/High | 9 | 10.38 | 6 | 5.26 | 0.31 | 0.046 |
| | | | | | (0.09, 1.04) | |
| Low/Low | 12 | 4.81 | 10 | 15.21 | 2.44 | 0.080 |
| | | | | | (0.87, 6.86) | |
| Low/High | 7 | 10.7 | 6 | 3.09 | 0.26 | 0.046 |
| | | | | | (0.06, 1.07) | |

Table 7 describes the comparison of overall survival between the Erlotinib and Placebo arms for various subsets of the patients in BR.21. In this table, a hazard ratio <1 indicates that the Erlotinib arm had superior survival to the Placebo arm, while a hazard ratio >1 indicates that the Erlotinib arm had inferior survival to the Placebo arm. P-values are determined from univariate Kaplan-Meier analyses. Hazard ratios with confidence limits are determined from Cox proportional hazards models.

TABLE 7B
Overall Survival, E-Cadherin High vs. E-Cadherin Low.
Table T_7B_Survival
Overall Survival by Treatment Arm Results for E-Cadherin

| | E-Cadherin | | E-Cadherin | | | |
|---|---|---|---|---|---|---|
| | High | | Low | | | |
| | | Median | | Median | Hazard Ratio | |
| | | Survival | | Survival | (H/L) | Log-Rank |
| Characteristics | N | Months | N | Months | (95% CI) | p-value |
| Erlotinib | 38 | 11.3 | 19 | 4.86 | 0.68 | 0.257 |
| | | | | | (0.35, 1.33) | |
| Placebo | 22 | 4.24 | 16 | 6.83 | 1.48 | 0.312 |
| | | | | | (0.69, 3.15) | |

Table 7B describes the comparison of overall survival between the E-Cadherin high and E-Cadherin low subsets for those patients in BR.21 who had evaluable E-Cadherin results (N=95). In this table, a hazard ratio <1 indicates that the High E-Cadherin subset had superior survival to the Low E-Cadherin subset, while a hazard ratio >1 indicates that the High E-Cadherin subset had inferior survival to the Low E-Cadherin subset. P-values are determined from univariate Kaplan-Meier analyses. Hazard ratios with confidence limits are determined from Cox proportional hazards models.

TABLE 7C
Overall Survival, Vimentin High vs. Vimentin Low.
Table T_7C_Survival
Overall Survival by Treatment Arm Results for Vimentin

| | Vimentin | | Vimentin | | | |
|---|---|---|---|---|---|---|
| | High | | Low | | | |
| | | Median | | Median | Hazard Ratio | |
| | | Survival | | Survival | (H/L) | Log-Rank |
| Characteristics | N | Months | N | Months | (95% CI) | p-value |
| Erlotinib | 16 | 10.55 | 41 | 6.11 | 0.65 | 0.255 |
| | | | | | (0.31, 1.38) | |
| Placebo | 12 | 3.56 | 26 | 5.42 | 2.32 | 0.025 |
| | | | | | (1.09, 4.94) | |

Table 7C describes the comparison of overall survival between the vimentin high and vimentin low subsets for those patients in BR.21 who had evaluable vimentin results (N=95). In this table, a hazard ratio <1 indicates that the High vimentin subset had superior survival to the Low vimentin subset, while a hazard ratio >1 indicates that the High vimentin subset had inferior survival to the Low vimentin subset. P-values are determined from univariate Kaplan-Meier analyses. Hazard Ratios are determined from univariate Cox proportional hazards models.

TABLE 8
Progression Free Survival, Erlotinib vs. Placebo.
Table T_8_PFS
PFS by E-Cadherin and Vimentin Results

| | Erlotinib | | Placebo | | | |
|---|---|---|---|---|---|---|
| | | Median | | Median | | Log- |
| | | PFS | | PFS | Hazard Ratio | Rank |
| Characteristics | N | Months | N | Months | (95% CI) | p-value |
| All Patients | 488 | 2.23 | 243 | 1.84 | 0.64 | <0.001 |
| | | | | | (0.54, 0.75) | |
| Patients with | 57 | 2.38 | 38 | 1.84 | 0.72 | 0.140 |
| Known | | | | | | |
| E-Cadherin | | | | | | |
| Results | | | | | | |
| | | | | | (0.47, 1.12) | |
| Patients with | 57 | 2.38 | 38 | 1.84 | 0.72 | 0.140 |
| Known | | | | | | |
| Vimentin | | | | | | |
| Results | | | | | | |
| | | | | | (0.47, 1.12) | |
| E-Cadherin | | | | | | |
| High | 38 | 3.68 | 22 | 1.81 | 0.52 | 0.021 |
| | | | | | (0.67, 1.87) | |
| Low | 19 | 1.97 | 16 | 2.09 | 1.18 | 0.646 |
| | | | | | (0.11, 0.70) | |
| Vimentin | | | | | | |
| Low | 41 | 2.18 | 26 | 1.91 | 1.11 | 0.681 |
| | | | | | (0.67, 1.87) | |
| High | 16 | 5.39 | 12 | 1.51 | 0.28 | 0.004 |
| | | | | | (0.11, 0.70) | |
| E-Cadherin/ | | | | | | |
| Vimentin | | | | | | |
| High/Low | 29 | 2.66 | 16 | 1.84 | 0.73 | 0.322 |
| | | | | | (0.38, 1.38) | |
| High/High | 9 | 8.02 | 6 | 1.35 | 0.235 | 0.025 |
| | | | | | (0.06, 0.93) | |
| Low/Low | 12 | 1.69 | 10 | 3.35 | 2.75 | 0.029 |
| | | | | | (1.06, 7.09) | |
| Low/High | 7 | 3.61 | 6 | 1.56 | 0.24 | 0.037 |
| | | | | | (0.05, 1.03) | |

Table 8 describes the comparison of progression free survival (PFS) between the Erlotinib and Placebo arms for various subsets of the patients in BR.21. In this table, a hazard ratio <1 indicates that the Erlotinib arm had superior PFS to the Placebo arm, while a hazard ratio >1 indicates that the Erlotinib arm had inferior PFS to the Placebo arm. P-values are determined from univariate Kaplan-Meier analyses. Hazard ratios with confidence limits are determined from Cox proportional hazards models.

TABLE 8B
Progression Free Survival, E-Cadherin High vs. E-Cadherin Low.
Table T_8B_PFS
Progression-Free Survival by Treatment Arm Results for E-Cadherin

| | E-Cadherin | | E-Cadherin | | | |
|---|---|---|---|---|---|---|
| | High | | Low | | | |
| | | Median | | Median | Hazard Ratio | |
| | | Survival | | Survival | (H/L) | Log-Rank |
| Characteristics | N | Months | N | Months | (95% CI) | p-value |
| Erlotinib | 38 | 3.68 | 19 | 1.97 | 0.57 | 0.060 |
| | | | | | (0.32, 1.03) | |
| Placebo | 22 | 1.81 | 16 | 2.09 | 1.34 | 0.399 |
| | | | | | (0.67, 2.66) | |

Table 8B describes the comparison of progression free survival (PFS) between the E-Cadherin high and E-Cadherin low subsets for those patients in BR.21 who had evaluable E-Cadherin results (N=95). In this table, a hazard ratio <1 indicates that the High E-Cadherin subset had superior PFS to the Low E-Cadherin subset, while a hazard ratio >1 indicates that the High E-Cadherin subset had inferior PFS to the Low E-Cadherin subset. P-values are determined from univariate Kaplan-Meier analyses. Hazard ratios with confidence limits are determined from Cox proportional hazards models.

TABLE 8C
Progression Free Survival, Vimentin High vs. Vimentin Low.
Table T_8C_PFS
Progression-Free Survival by Treatment Arm Results for Vimentin

| | Vimentin | | Vimentin | | | |
|---|---|---|---|---|---|---|
| | High | | Low | | | |
| | | Median | | Median | Hazard Ratio | |
| | | Survival | | Survival | (H/L) | Log-Rank |
| Characteristics | N | Months | N | Months | (95% CI) | p-value |
| Erlotinib | 16 | 5.39 | 41 | 2.18 | 0.43 | 0.013 |
| | | | | | (0.22, 0.85) | |
| Placebo | 12 | 1.51 | 26 | 1.91 | 2.07 | 0.59 |
| | | | | | (0.94, 4.55) | |

Table 8C describes the comparison of progression free survival (PFS) between the vimentin high and vimentin low subsets for those patients in BR.21 who had evaluable vimentin results (N=95). In this table, a hazard ratio <1 indicates that the High vimentin subset had superior PFS to the Low vimentin subset, while a hazard ratio >1 indicates that the High vimentin subset had inferior PFS to the Low vimentin subset. P-values are determined from univariate Kaplan-Meier analyses. Hazard ratios with confidence limits are determined from Cox proportional hazards models.

Cut-Point Analyses for Survival and PFS for E-Cadherin and Vimentin:

In order to determine the optimal cut-point for High and Low for E-Cadherin analyses, multiple analyses were performed at each of a large number of cut points for each scoring method. Only those cut points for which there was sufficient data were considered, with sufficient defined as at least 10 patients in each group, and each group being 20-80% of the size of the total number of evaluable patients.

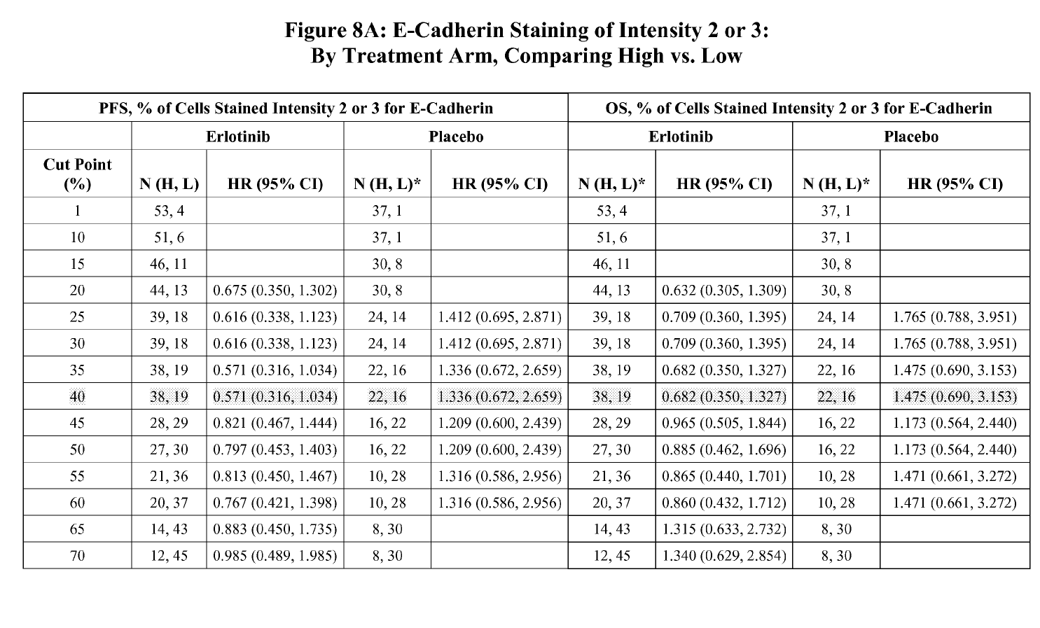

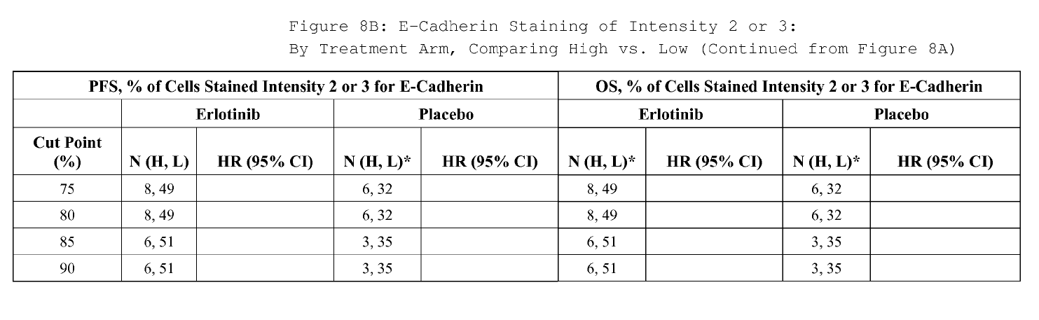

In the table of FIG. 8, which uses Staining of Intensity +2 or +3 as the metric, analyses were generated at cut points of 1, 5, 10, and increasing by 5 up to 95. A cut-point of 40% provides differentiation in the Erlotinib arm (HR=0.571 for progression free survival and 0.682 for overall survival). In addition, discussions with the in-house pathologist confirmed that this was a cut-point with practical utility. The combination of statistical differentiation and clinical relevance drove the choice of 40% staining of intensity +2 or +3.

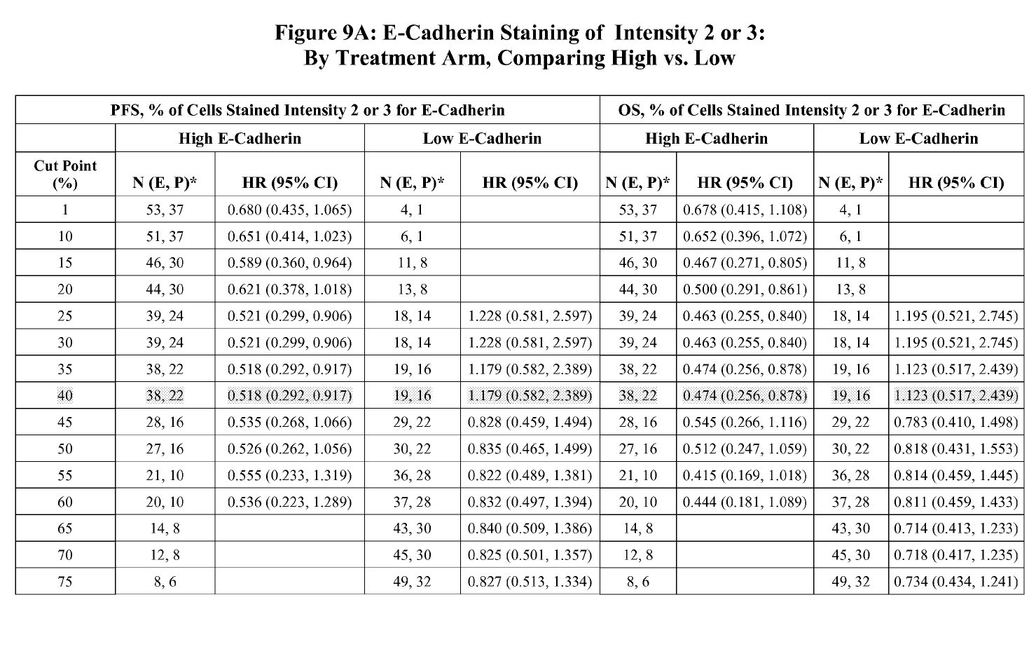

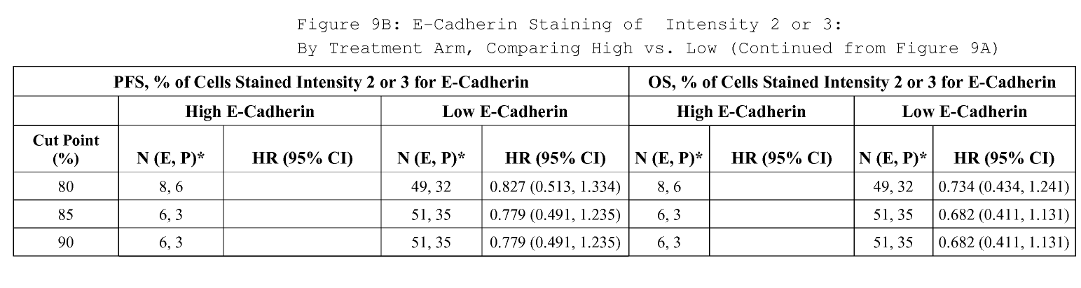

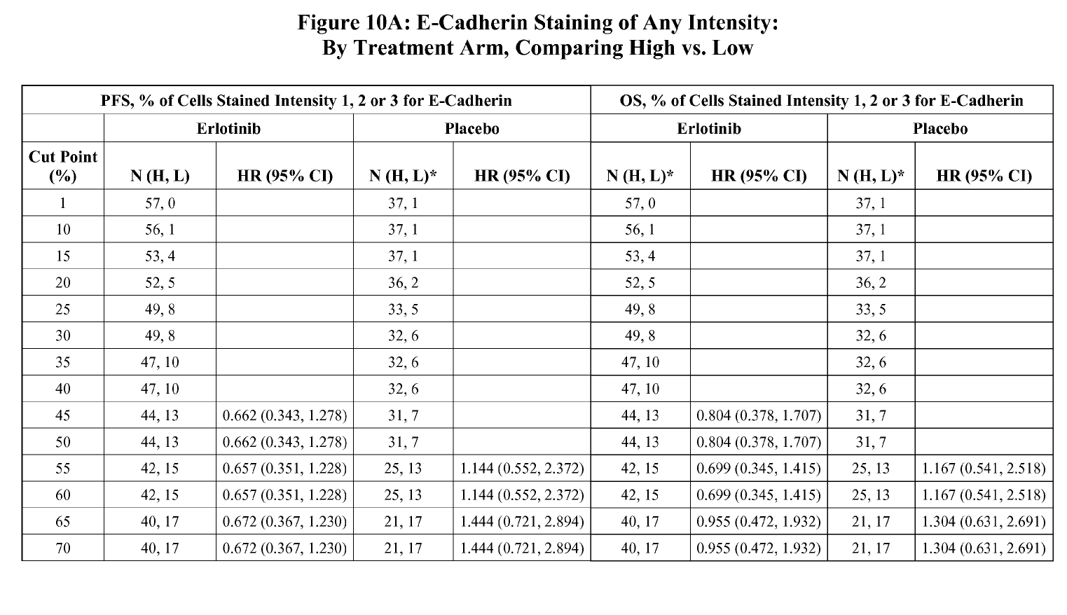

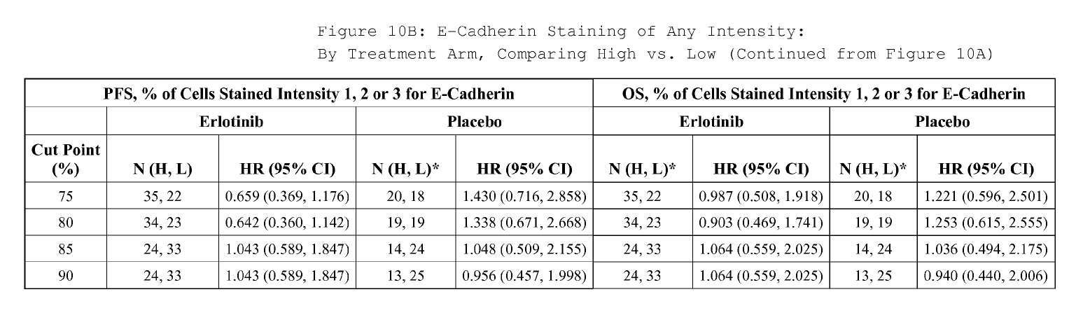

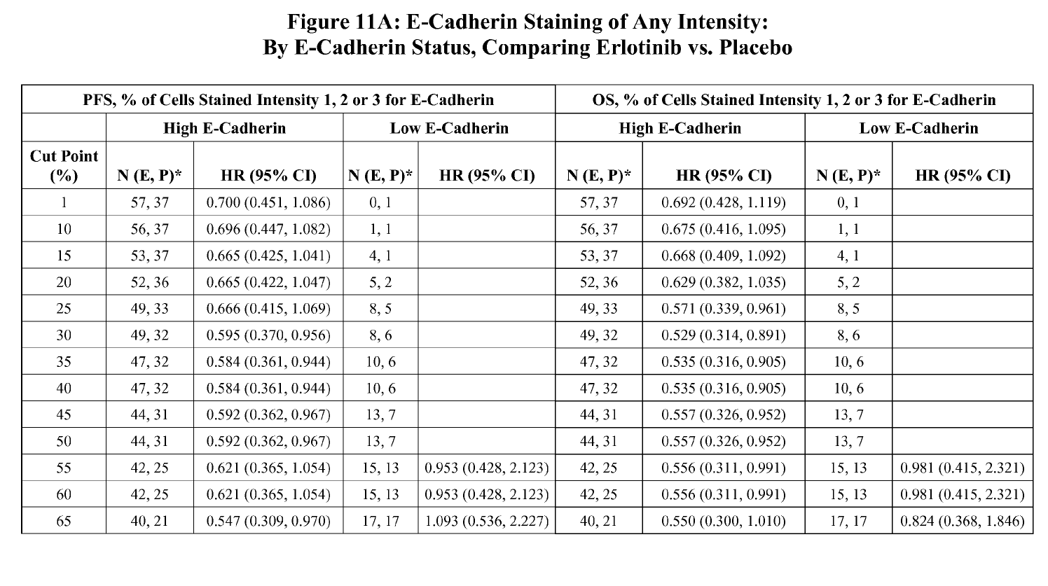

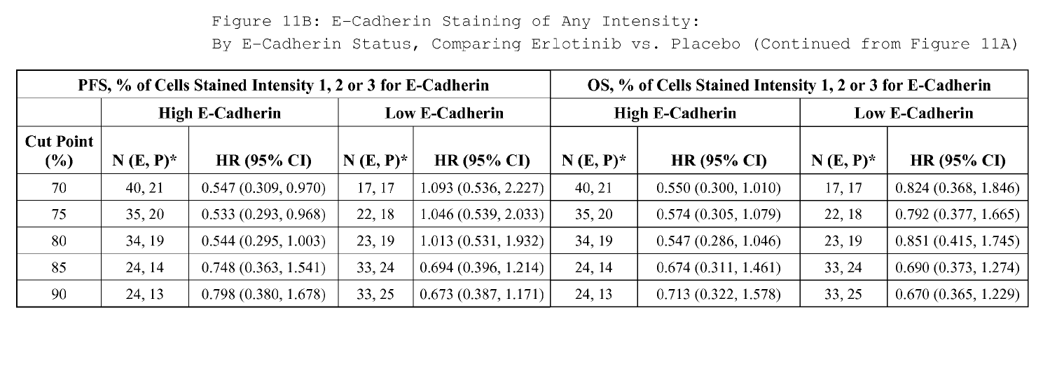

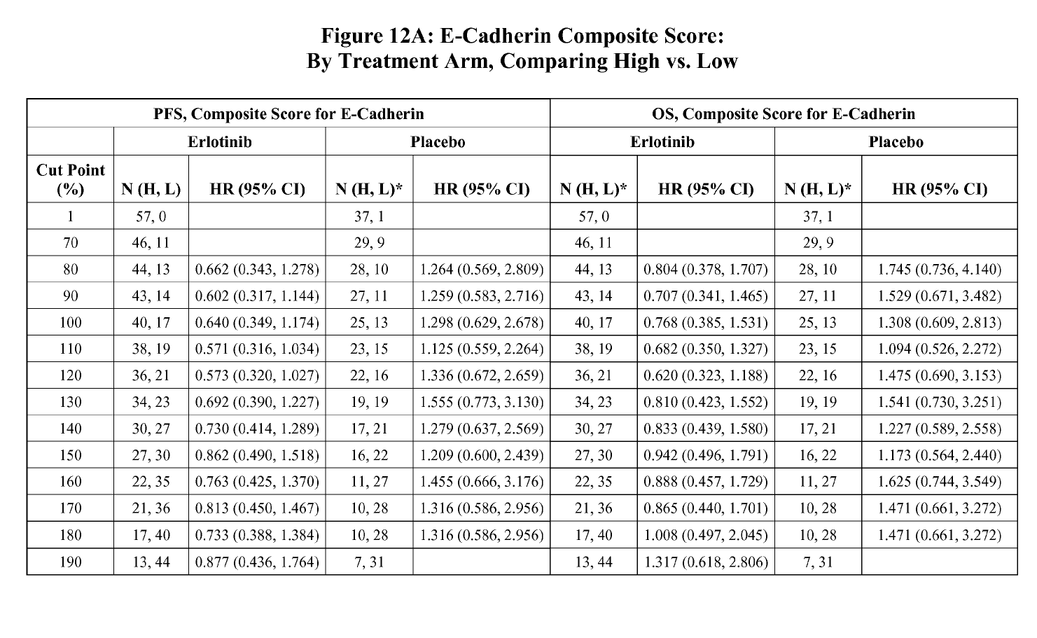

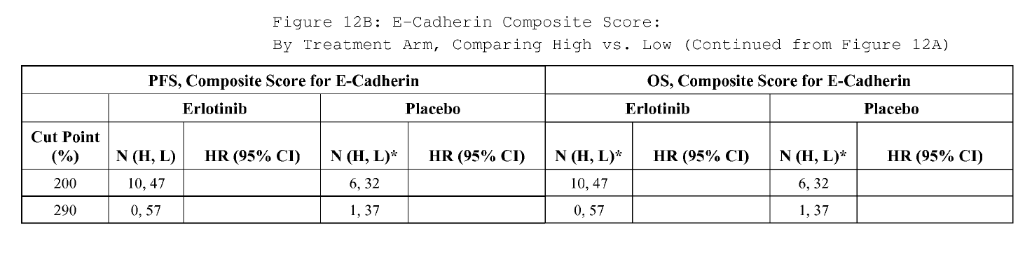

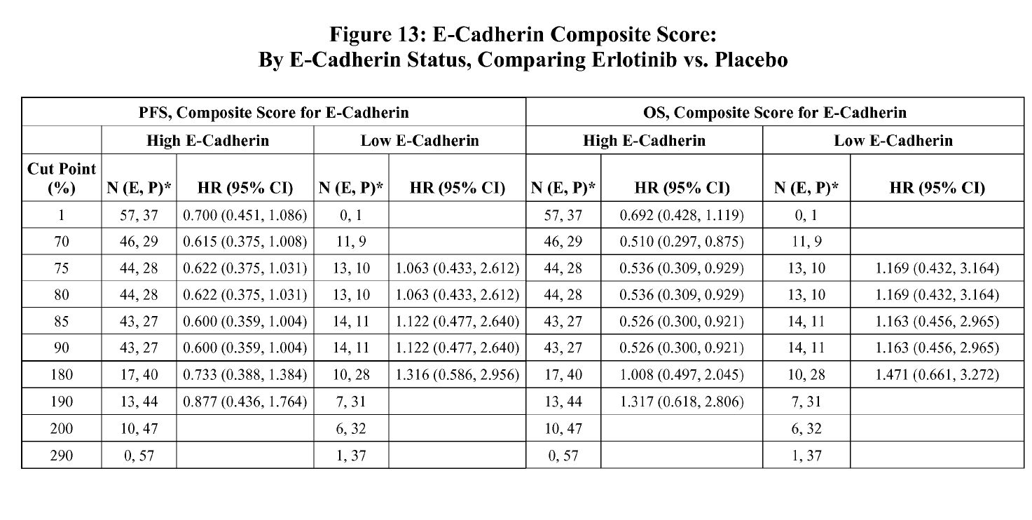

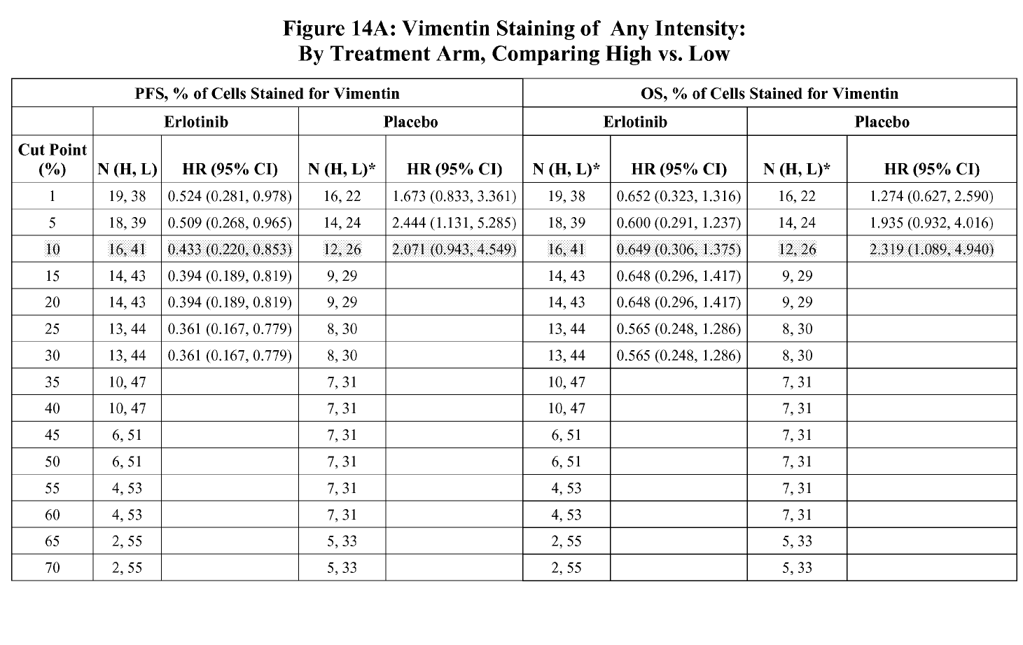

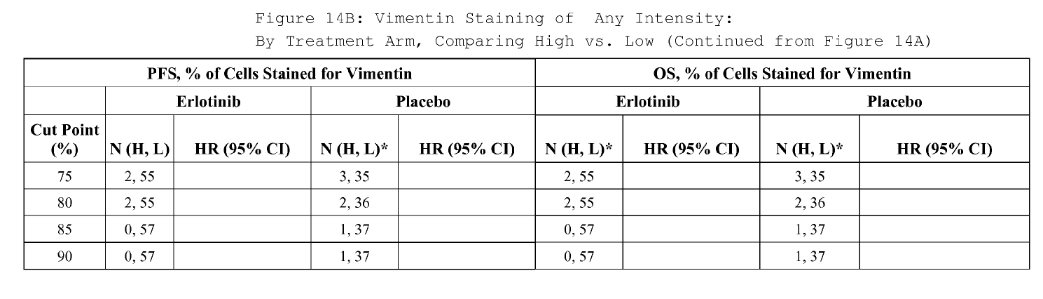

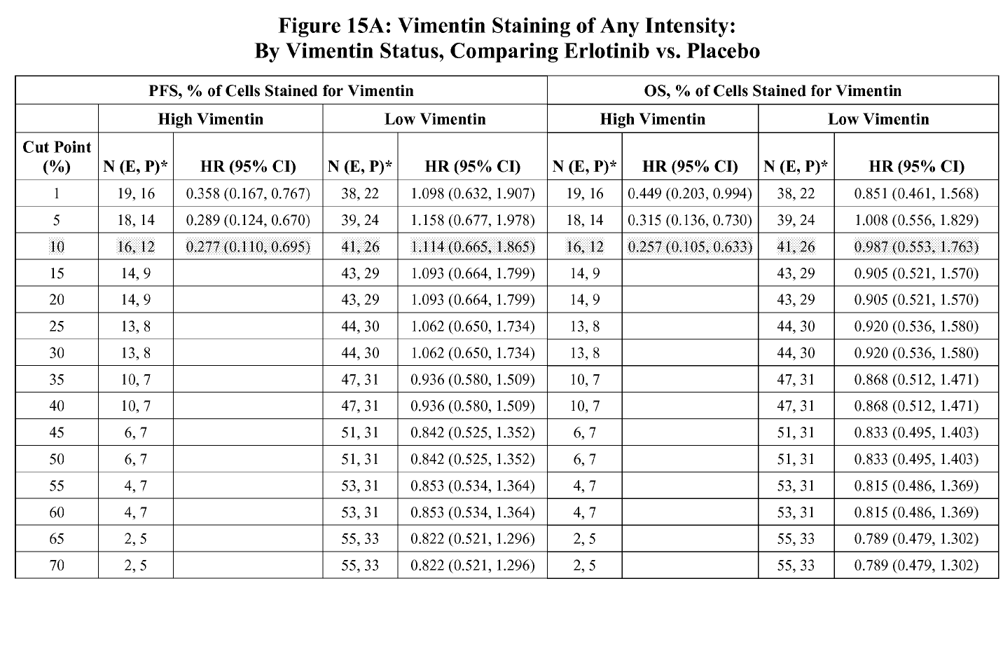

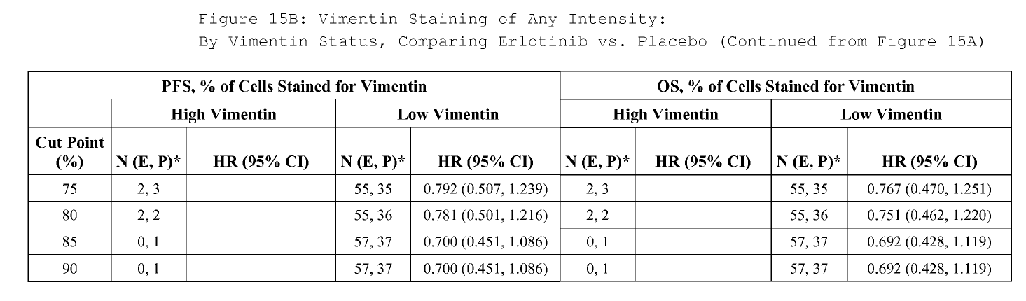

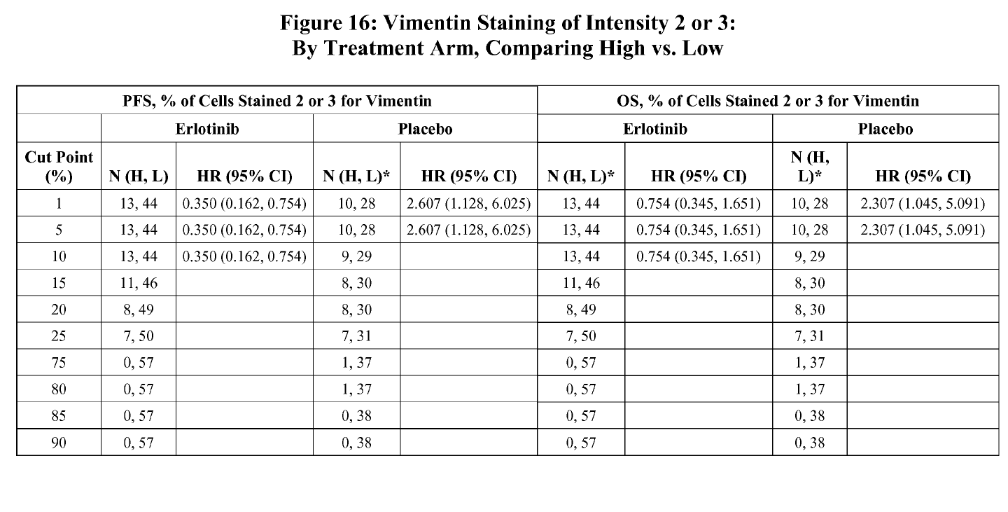

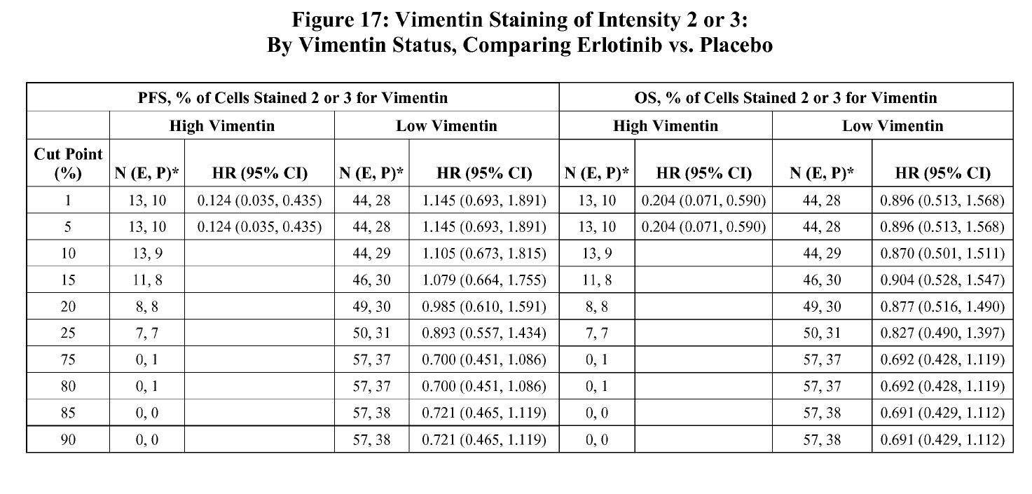

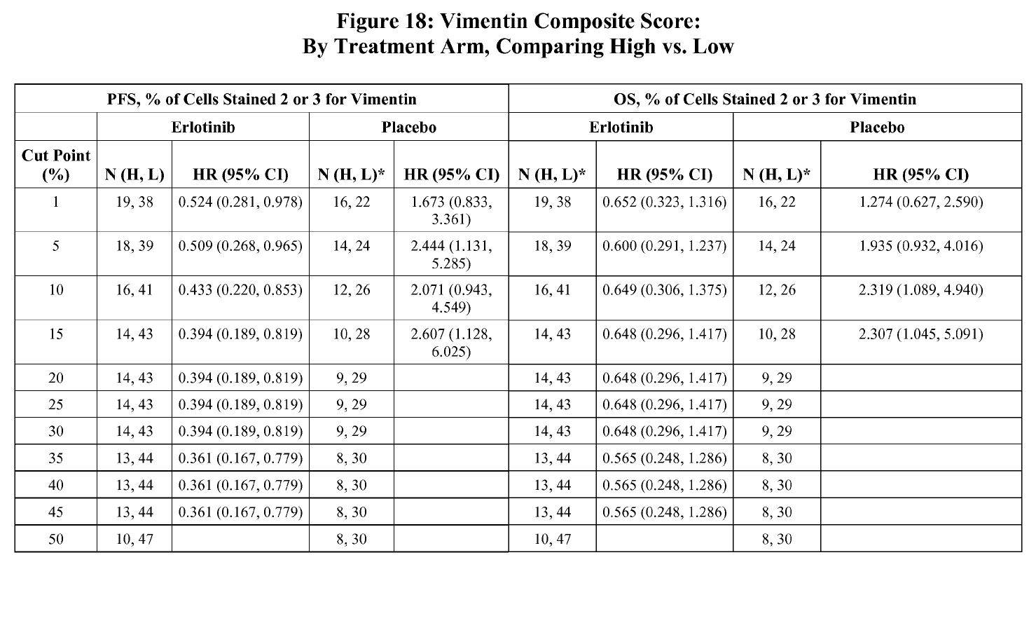

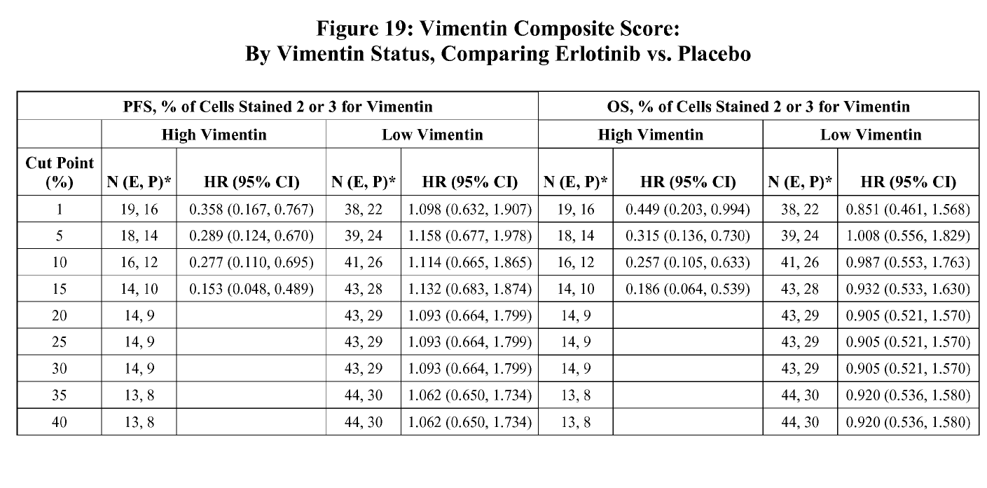

Similar tables were generated for the two other metrics, staining of any intensity (+1, +2, or +3) as well as composite score (defined previously), for both E-Cadherin and vimentin (FIGS. 8-19).

Summary of Results

Of 163 tissue samples received from NCIC (22% of all patients), 95 patients had an evaluable E-Cadherin slide and the same 95 patients had an evaluable vimentin slide. Therefore results were obtained on 58% of the 163 tissue samples.

Pretreatment characteristics (demographics, prior therapies, disease characteristics) in the patients with evaluable tissue results were generally similar to the overall population for the study (Tables 3-5).

Based on the sensitivity analyses, a cut-point of 40% staining of intensity +2 or +3 was chosen for E-Cadherin. Using this cut point, 63% of evaluable patients were classified as High E-Cadherin and 37% were classified as Low E-Cadherin (Table 6).

Based on the sensitivity analyses, a cut-point of 10% staining of any intensity was chosen for Vimentin. Using this cut point, 29% of evaluable patients were classified as High vimentin and 71% were classified as Low vimentin (Table 6).

Tumor Expression E-Cadherin and vimentin in the patient population was as follows (see Table 6):

16% were High for both E-Cadherin and Vimentin.

14% were Low for E-Cadherin and High for Vimentin.

47% were High for E-Cadherin and Low for Vimentin.

23% were Low for E-Cadherin and Low for Vimentin

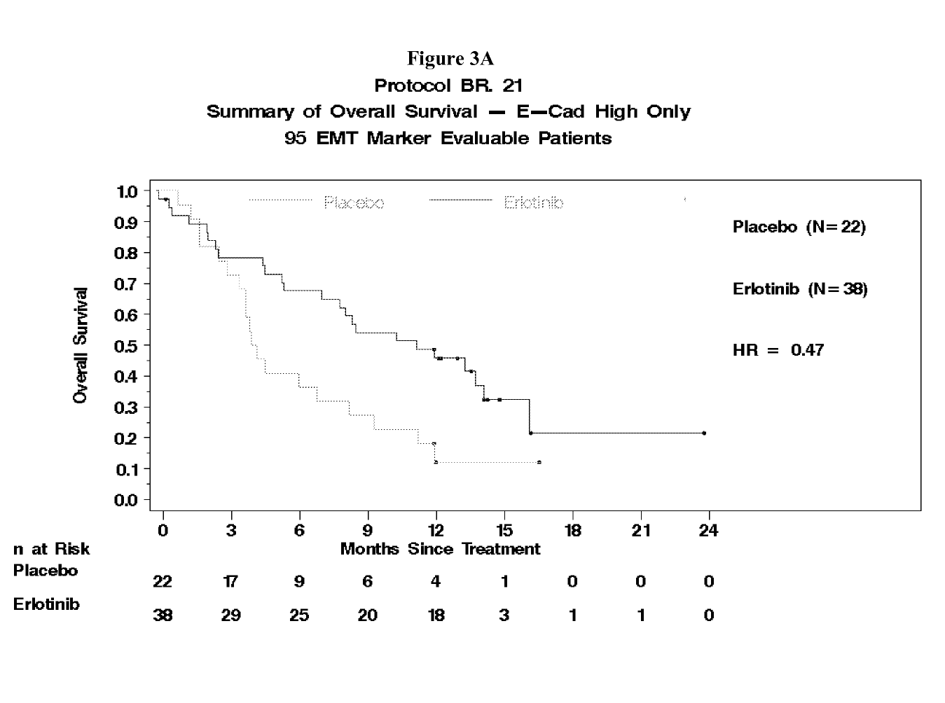

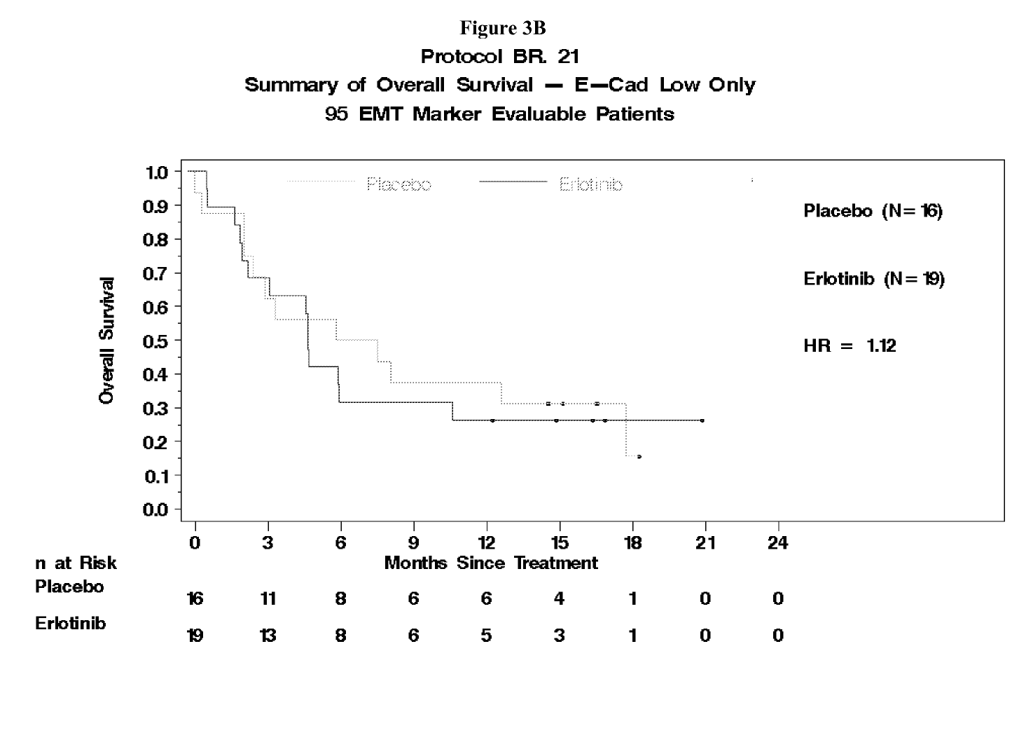

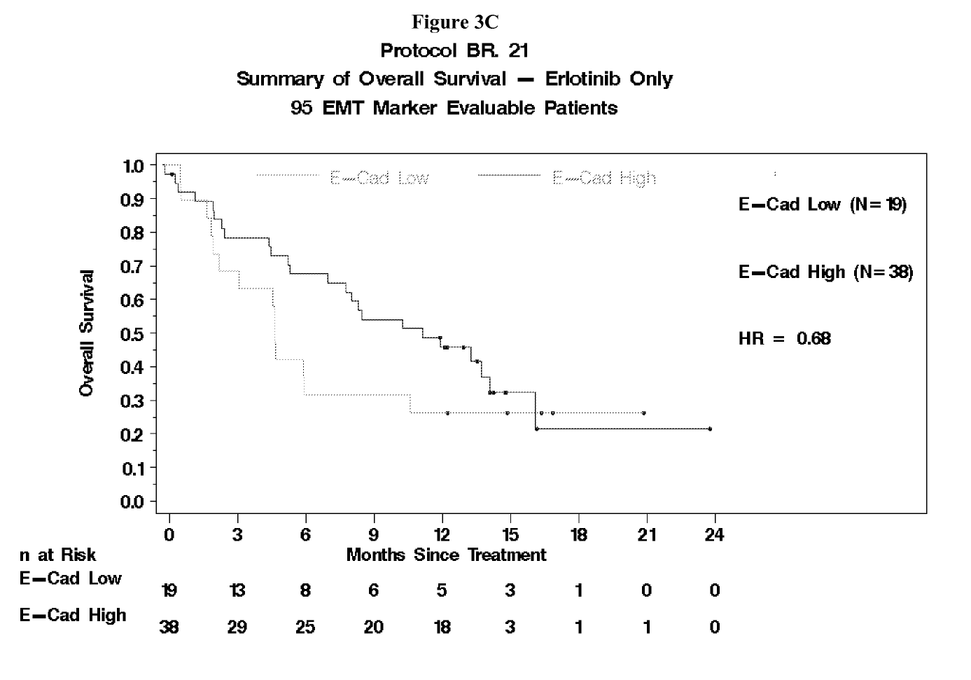

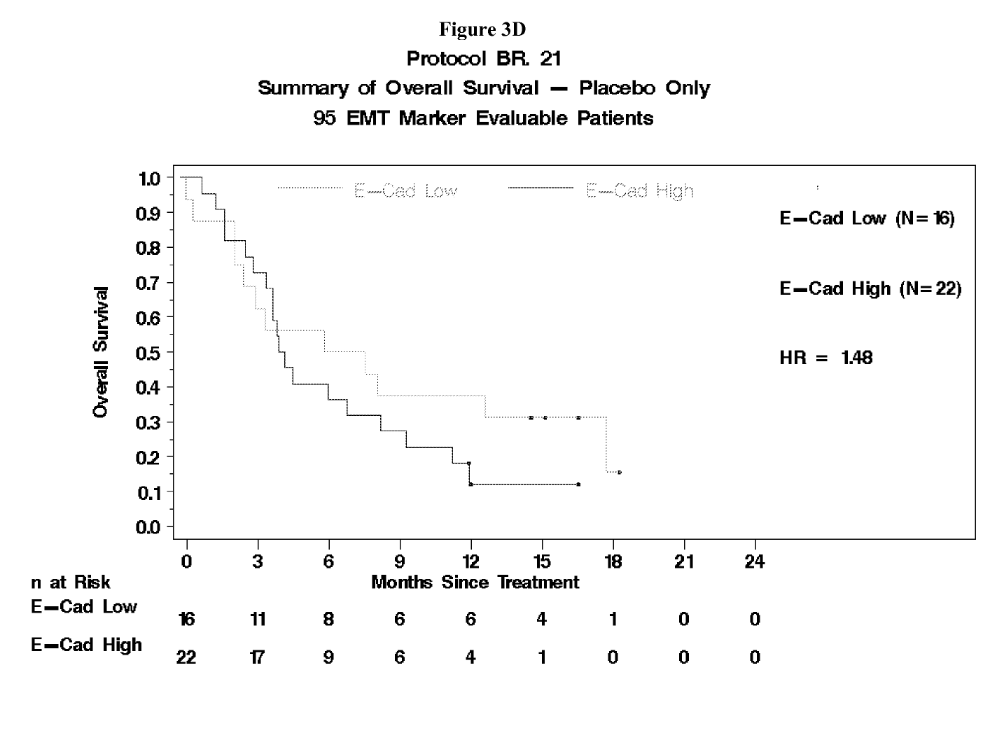

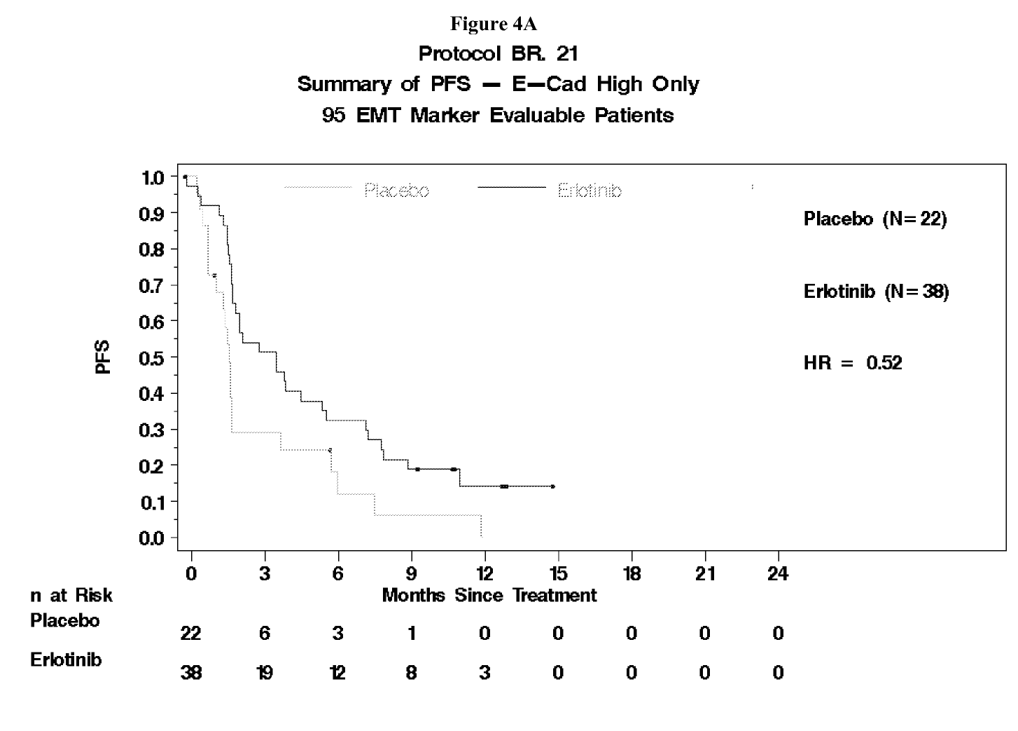

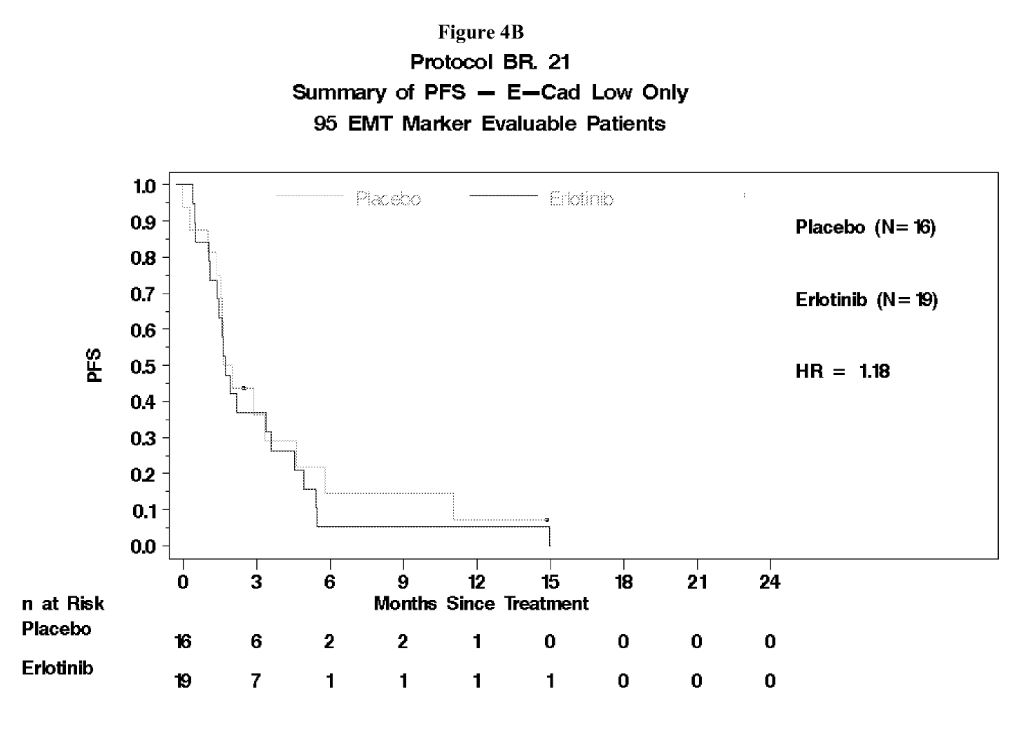

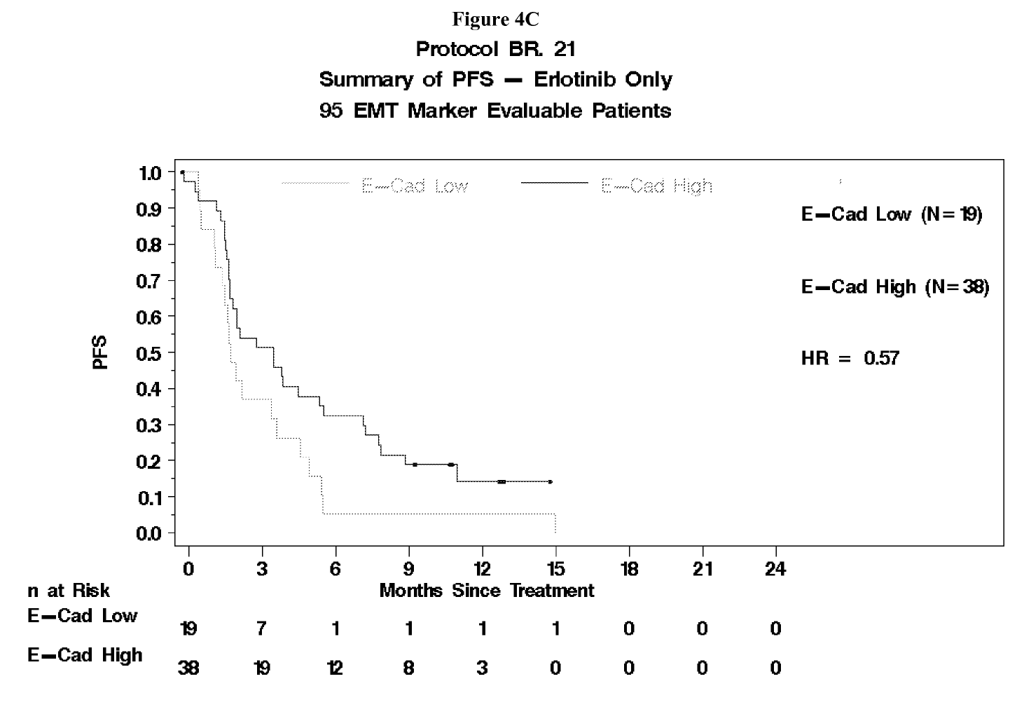

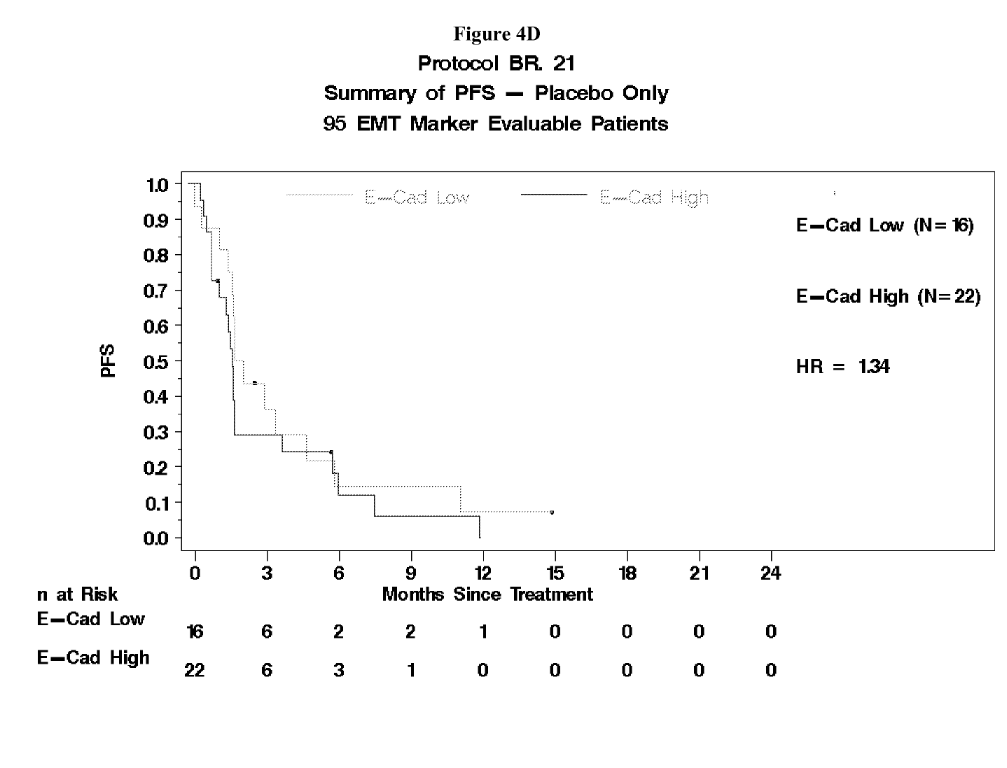

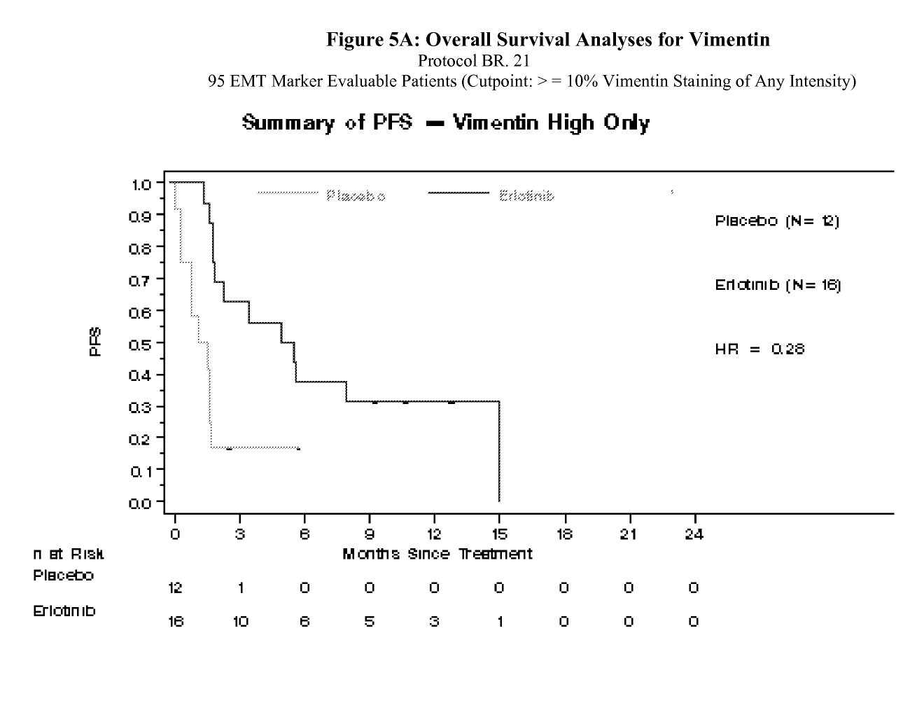

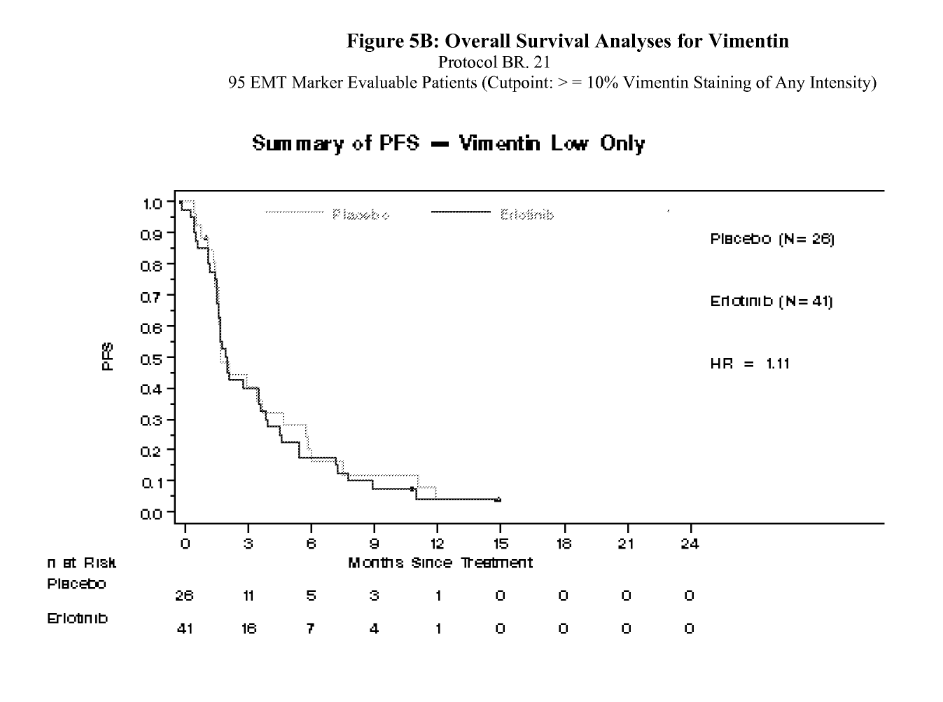

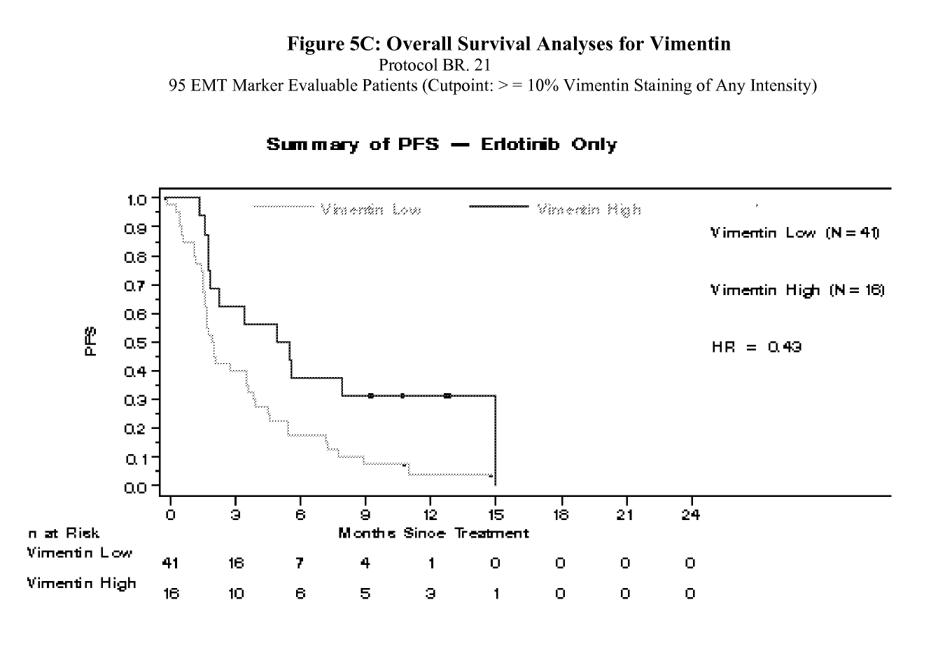

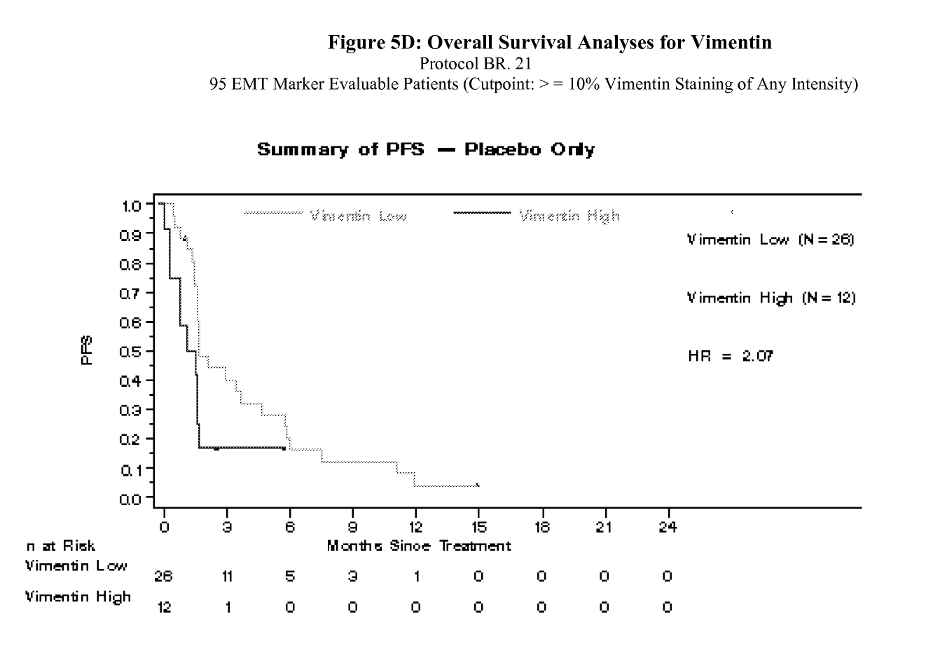

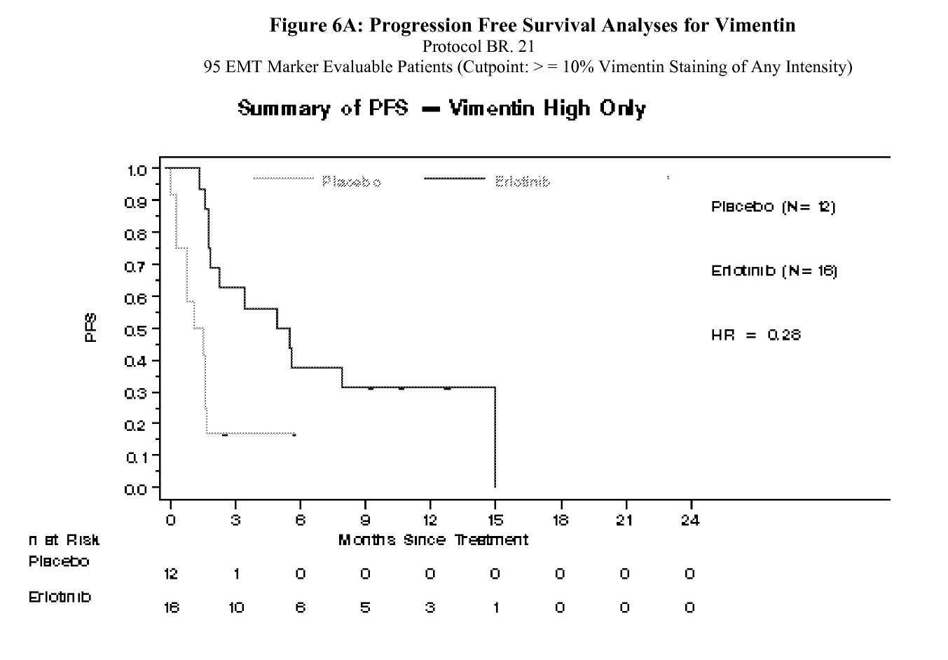

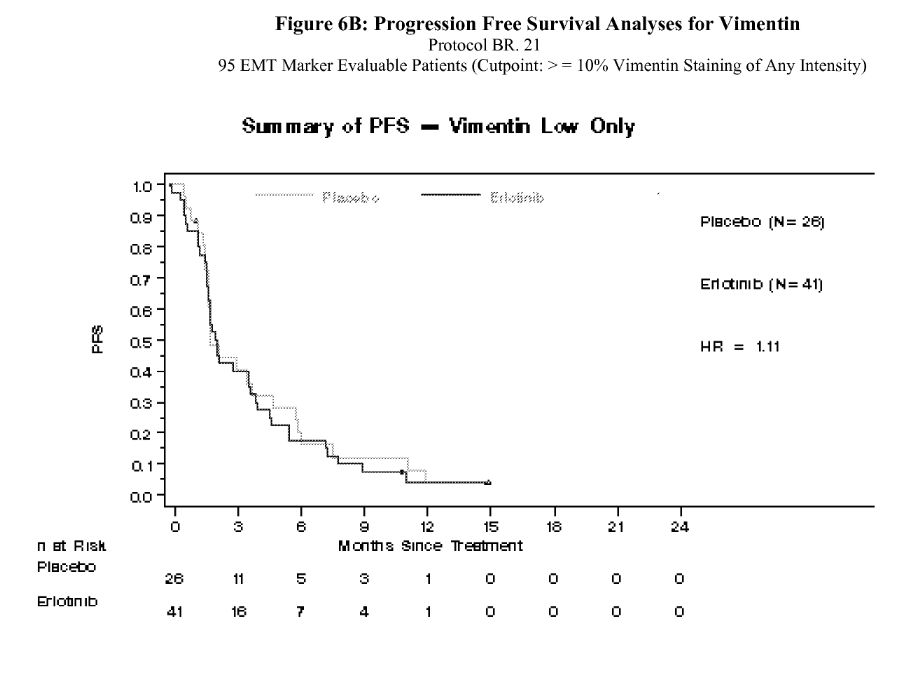

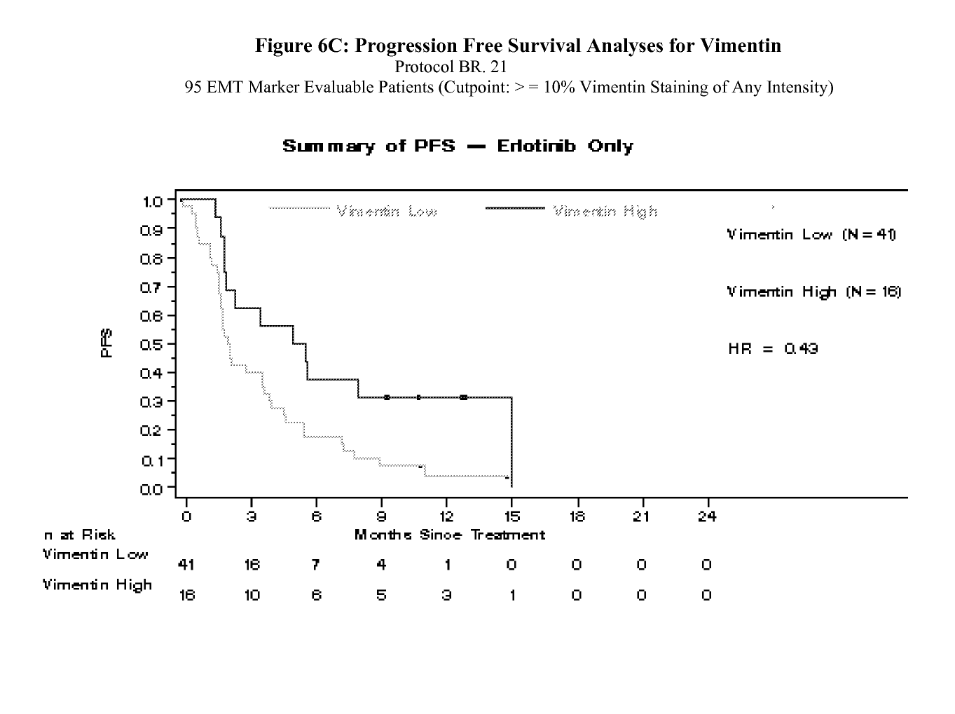

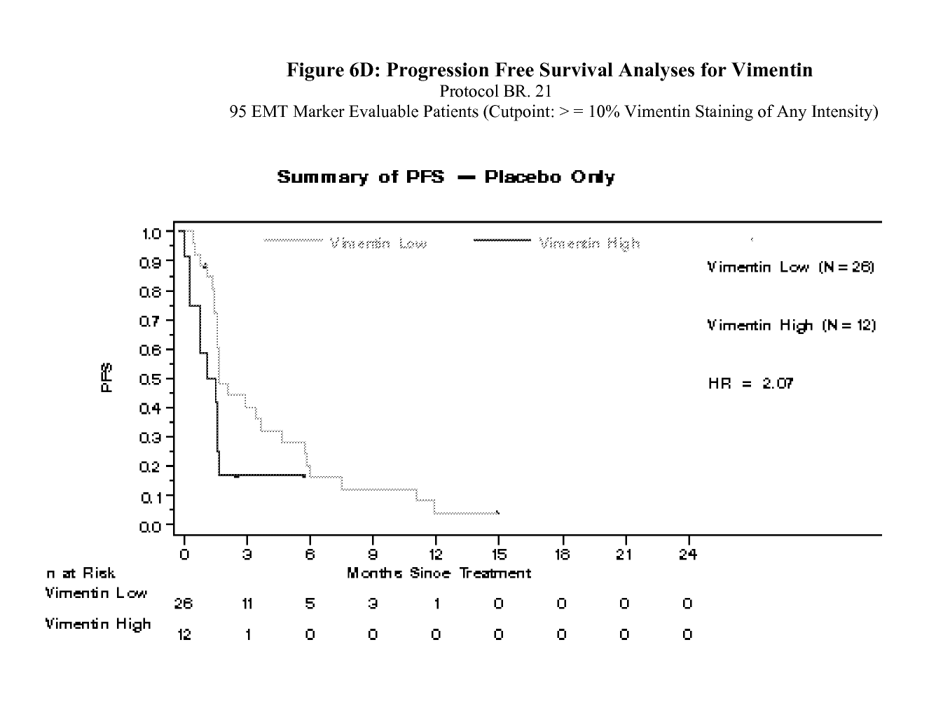

Overall survival and PFS (Table 7, 8, and K-M plots, FIGS. 3-6):

Survival outcomes in patients with evaluable biomarker results were similar to survival observed in the overall study population (HR=0.76 in overall population vs. HR=0.69 in EMT marker-evaluable subset).

PFS outcomes in patients with evaluable biomarker results were similar to PFS observed in the overall study population (PFS HR=0.64 in overall population vs. HR=0.72 in EMT marker-evaluable subgroup).

E-Cadherin results in the Erlotinib arm provide a cut-point which associates with better outcome in the high E-Cadherin expression group. In the Placebo arm, the effects were in the opposite direction, which suggest that high E-Cadherin may be a poor prognostic but a good predictive factor.

High vimentin expressing patients appear to have longer overall survival and progression-free survival than low vimentin expressing patients in the Erlotinib arm.

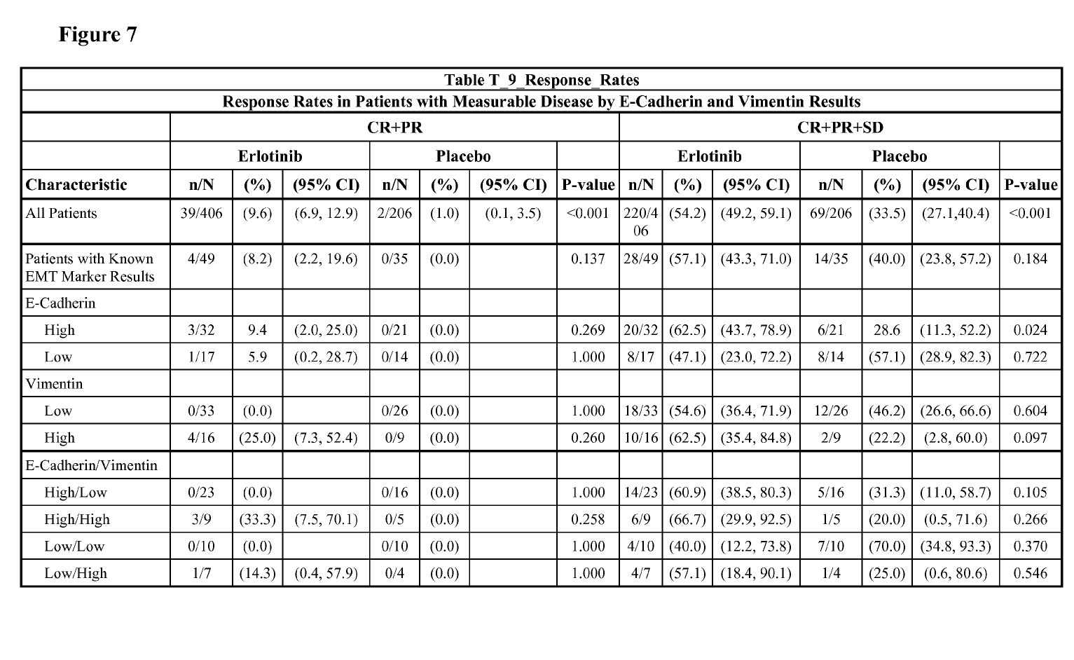

FIG. 7 presents response and disease control rates by E-Cadherin and vimentin status. Plots for survival and PFS are presented in FIGS. 3-6.

### CONCLUSIONS

Briefly, the results demonstrated the following: 1) the remaining tissue samples represented the overall BR21 patient population demographically, histologically and in treatment outcomes, 2) the subset of TARCEVA® treated patients with high E-cadherin expression, assessed as described in the Materials and Methods, demonstrated longer survival when treated with TARCEVA® 3) the subset of TARCEVA® treated patients with high vimentin expression, assessed as described in the Materials and Methods, demonstrated significantly longer survival when treated with TARCEVA®, 4) the effect for either marker was not observed in the placebo population. The results indicate that 2^{nd} and 3^{rd} line NSCLC patients whose tumors express high levels of vimentin and/or E-cadherin protein were associated with enhanced benefit from TARCEVA®.

### ABBREVIATIONS

HR, hazard ratio; PFS, progression free survival; OS, overall survival; CI, confidence interval; E, erlotinib; P, placebo; H, high; L, low; EGF, epidermal growth factor; EMT, epithelial to mesenchymal transition; NSCLC, non-small cell lung carcinoma; HNSCC, head and neck squamous cell carcinoma; CRC, colorectal cancer; MBC, metastatic breast cancer; EGFR, epidermal growth factor receptor; LC, liquid chromatography; MS, mass spectrometry; IGF-1, insulin-like growth factor-1; TGFα, transforming growth factor alpha; HB-EGF, heparin-binding epidermal growth factor; TGFα, transforming growth factor alpha; IC₅₀, half maximal inhibitory concentration; pY, phosphotyrosine; wt, wild-type; PI3K, phosphatidyl inositol-3 kinase; GAPDH, Glyceraldehyde 3-phosphate dehydrogenase.

### INCORPORATION BY REFERENCE

All patents, published patent applications and other references disclosed herein are hereby expressly incorporated herein by reference.

### EQUIVALENTS

Those skilled in the art will recognize, or be able to ascertain, using no more than routine experimentation, many equivalents to specific embodiments of the invention described specifically herein. Such equivalents are intended to be encompassed in the scope of the following claims.

What is claimed is:

1. A method of predicting the effectiveness of treatment of a cancer patient with an EGFR kinase inhibitor, comprising: assessing the level of the biomarker E-cadherin expressed by cells of a tumor of the patient; determining whether the tumor expresses high or low expression levels of E-cadherin; and predicting the effectiveness of treatment, wherein a high level of E-cadherin indicates that treatment will be more effective; and wherein the effectiveness of treatment of the cancer patient is indicated by either a longer overall survival or longer progression free survival in response to treatment.

2. A method for treating a patient with cancer, comprising: a step of predicting the effectiveness of treatment of a cancer patient with an EGFR kinase inhibitor, by assessing the level of the biomarker E-cadherin expressed by cells of a tumor of the patient; determining whether the tumor expresses high or low expression levels of E-cadherin; and predicting the effectiveness of treatment, wherein a high level of E-cadherin indicates that treatment will be more effective; and wherein the effectiveness of treatment of the cancer patient is indicated by either a longer overall survival or longer progression free survival in response to treatment; and a step of administering the patient a therapeutically effective dose of an EGFR kinase inhibitor.

3. A method for treating a patient with cancer, comprising administering to the patient a therapeutically effective dose of an EGFR kinase inhibitor if it is predicted that the patient will have a longer overall survival or longer progression free survival in response to the treatment by virtue of having tumor cells that express high levels of the biomarker E-cadherin.

4. The method of claim 1, 2 or 3, wherein the tumor cells are non-small cell lung cancer, pancreatic cancer, breast cancer, head and neck cancer, gastric cancer, colon cancer, or ovarian cancer.

5. The method of claim 1, 2 or 3, wherein the EGFR kinase inhibitor is erlotinib, gefitinib, canertinib, vandetanib, cetuximab, panitumumab, or matuzumab.

6. The method of claim 1, 2 or 3, wherein E-cadherin expression level is assessed by measuring E-cadherin protein.

7. The method of claim 6, wherein E-cadherin expression level is assessed by immunohistochemistry.

8. The method of claim 7, wherein E-cadherin expression level is determined by use of a standardized scoring system.

9. The method of claim 8, wherein a high E-cadherin expression level is indicated by 40% or more of the tumor cells having a staining intensity of +2 or +3 for E-cadherin.

10. The method of claim 1, 2 or 3, wherein E-cadherin expression level is assessed by measuring E-cadherin mRNA.
